(19) United States
(12) Patent Application Publication
(10) Pub. No.: US 2021/0332131 A1
(43) Pub. Date: Oct. 28, 2021

(54) COMPOSITIONS AND METHODS COMPRISING IgA ANTIBODY CONSTRUCTS

(71) Applicants: TigaTX, Inc., Westerly, RI (US); UMC Utrecht Holding B.V., Utrecht (NL)

(72) Inventors: Mark De Boer, Westerly, RI (US); Jeannette Henrica Wilhelmina LEUSEN, Utrecht (NL); Geert Jan VAN TETERING, Utrecht (NL)

(21) Appl. No.: 17/242,793

(22) Filed: Apr. 28, 2021

Related U.S. Application Data
(63) Continuation of application No. PCT/US2019/058648, filed on Oct. 29, 2019.
(60) Provisional application No. 62/752,641, filed on Oct. 30, 2018.

(30) Foreign Application Priority Data
Oct. 29, 2019 (EP) 18203183.1

(51) Int. Cl. C07K 16/28

(52) U.S. Cl. CPC C07K 16/2803; C07K 16/2887; C07K 2317/732; C07K 2317/622; C07K 2317/31

(57) ABSTRACT

Provided herein are therapeutic agents, pharmaceutical compositions, and methods comprising an IgA constant region. The compositions and methods described herein facilitate binding of an antibody construct comprising an IgA constant domain to CD47 and an antigen for instance a tumor related antigen such as CD20 or CD19.

### CROSS-REFERENCE

This application is a Continuation Application of International Patent Application PCT/US2019/058648, filed Oct. 29, 2019, which claims the benefit of European Patent Application EP 18203183.1, filed Oct. 29, 2018, and U.S. Provisional Application No. 62/752,641, filed Oct. 30, 2018; each of which application is incorporated herein by reference in their entirety.

### SEQUENCE LISTING

The instant application contains a Sequence Listing which has been submitted electronically in ASCII format and is hereby incorporated by reference in its entirety. Said ASCII copy, created on Dec. 2, 2019, is named 55207-704_601_SL.txt and is 97,536 bytes in size.

### BACKGROUND OF THE DISCLOSURE

Monoclonal antibodies of IgG isotype targeting tumor antigens have proven to be an effective treatment of various malignancies. Over the years an increasing number of monoclonal antibodies targeting different tumor antigens have been approved for use in cancer therapies. However, their clinical efficacy, especially in monotherapy, is still not sufficient. It is thereof of interest to develop new, alternative antibody therapies with increased clinical efficacy.

### SUMMARY OF THE DISCLOSURE

In one aspect provided herein is an antibody construct comprising; (a) an immunoglobulin A (IgA) heavy chain domain, (b) a CD47 binding domain, and (c) an antigen binding domain, wherein the IgA heavy chain domain specifically binds a FcαR on an immune effector cell, wherein the CD47 binding domain inhibits binding of a CD47 expressed on a target cell with a signal regulatory protein α (SIRP α) on the immune effector cell, wherein the antigen binding domain binds an antigen on the target cell, and wherein the antibody construct has a higher binding affinity for the antigen compared to the CD47.

In some embodiments, the CD47 binding domain comprises at least one of a first light chain variable domain and a first heavy chain variable domain. In some embodiments, the antigen binding domain comprises at least one of a second light chain variable domain and a second heavy chain variable domain. In some embodiments, said construct comprises said first light chain variable domain, and wherein said first light chain variable domain comprises a variable light chain complementarity determining region 1 (CDR-L1), a variable light chain complementarity determining region 2 (CDR L2), and a variable light chain complementarity determining region 3 (CDR-L3), wherein said CDR-L1 comprises an amino acid sequence of SEQ ID No: 13 or a variant thereof with up to two amino modifications in SEQ ID No: 13, wherein said CDR-L2 comprises an amino acid sequence of SEQ ID No: 14 or a variant thereof with up to two amino modifications in SEQ ID No: 14, and wherein said CDR-L3 comprises an amino acid sequence of SEQ ID No: 15 or a variant thereof with up to two amino modifications in SEQ ID No: 15. In some embodiments, construct comprises said first light chain variable domain, and wherein said first light chain variable domain comprises an amino acid sequence with 90% sequence identity to SEQ ID NO: 26.

In some embodiments, said construct comprises said first heavy chain variable domain, and wherein said first heavy chain variable domain comprises a variable heavy chain complementarity determining region 1 (CDR-H1), a variable heavy chain complementarity determining region 2 (CDR-H2), and a variable heavy chain complementarity determining region 3 (CDR-H3), wherein said CDR-H1 comprises an amino acid sequence of SEQ ID No: 4 or a variant thereof with up to two amino modifications in SEQ ID No: 4, wherein said CDR-H2 comprises an amino acid sequence of SEQ ID No: 5 or a variant thereof with up to two amino modifications in SEQ ID No: 5, and wherein said CDR-H3 comprises an amino acid sequence of SEQ ID No: 6 or a variant thereof with up to two amino modifications in SEQ ID No: 6.

In some embodiments, said construct comprises said first heavy chain variable domain and wherein the first heavy chain variable domain comprises an amino acid sequence with 90% sequence identity to SEQ ID NO: 23.

In some embodiments, said construct comprises said second light chain variable domain, and wherein said second light chain variable domain comprises a CDR-L1, a CDR L2, and a CDR-L3, wherein said CDR-L1 comprises an amino acid sequence of SEQ ID No: 10 or a variant thereof with up to two amino modifications in SEQ ID No: 10, wherein said CDR-L2 comprises an amino acid sequence of SEQ ID No: 11 or a variant thereof with up to two amino modifications in SEQ ID No: 11, and wherein said CDR-L3 comprises an amino acid sequence of SEQ ID No: 12 or a variant thereof with up to two amino modifications in SEQ ID No: 12. In some embodiments, said construct comprises said second light chain variable domain, and wherein said second light chain variable domain comprises an amino acid sequence of SEQ ID NO: 25.

In some embodiments, said construct comprises said second heavy chain variable domain, and said second heavy chain variable domain comprises a CDR-H1, a CDR-H2 and a CDR-H3, wherein said CDR-H1 comprises an amino acid sequence of SEQ ID No: 1 or a variant thereof with up to two amino modifications in SEQ ID No: 1, wherein said CDR-H2 comprises an amino acid sequence of SEQ ID No: 2 or a variant thereof with up to two amino modifications in SEQ ID No: 2, and wherein said CDR-H3 comprises an amino acid sequence of SEQ ID No: 3 or a variant thereof with up to two amino modifications in SEQ ID No: 3.

In some embodiments, said construct comprises said second heavy chain variable domain, and wherein said second heavy chain variable domain comprises an amino acid sequence with 90% sequence identity to SEQ ID NO:22.

In some embodiments, the antibody construct comprising at least one of, a first polypeptide, that comprises said first heavy chain variable domain and second heavy chain variable domain wherein the C-terminus of said second heavy chain variable domain is linked to the N-terminus of the first heavy chain variable domain; and a second polypeptide that comprises said first light chain variable domain and second light chain variable domain wherein the C-terminus of the second light chain variable domain is linked to the N-terminus of the first light chain variable domain.

In some embodiments, said first or said second polypeptide further comprise a linker peptide that links said variable domains. In some embodiments, the linker peptide comprises a sequence of SEQ ID NO:44. In some embodiments, the first polypeptide comprises a sequence of SEQ ID NO: 29. In some embodiments, the C-terminus of the first polypeptide is linked to the (IgA) heavy chain region. In some embodiments, the linking is via a IgA CH1 constant domain.

In some embodiments, the second polypeptide comprises a sequence of SEQ ID NO: 31. In some embodiments, the antibody construct comprises the first polypeptide and the second polypeptide. In some embodiments, the antibody construct comprising at least one of; a first polypeptide, wherein the C-terminus of the first heavy chain variable domain is linked to the N-terminus of the second heavy chain variable domain; or a second polypeptide, wherein the C-terminus of the first light chain variable domain is linked to the N-terminus of the second light chain variable domain.

In some embodiments, the linking is by a linker peptide. In some embodiments, the linker peptide comprises a sequence of SEQ ID NO: 44. In some embodiments, the first polypeptide comprises a sequence of SEQ ID NO: 30. In some embodiments, the C-terminus of the first polypeptide is linked to the (IgA) heavy chain region.

In some embodiments, the linking is via a IgA CH1 constant domain. In some embodiments, the second polypeptide comprises a sequence of SEQ ID NO: 32. In some embodiments, the antibody construct comprises the first polypeptide and the second polypeptide. In some embodiments, the antibody construct comprises a first polypeptide comprising a single chain variable fragment (scFv), wherein the scFV comprises said second heavy chain variable domain linked to said second light chain variable domain. In some embodiments, the construct comprising a linker peptide that links said variable domains. In some embodiments, the linker peptide comprises a sequence of SEQ ID NO 45. In some embodiments, the first polypeptide comprises the scFv linked to the first light chain variable domain; or the first heavy chain variable domain. In some embodiments, the linking is by a linker peptide. In some embodiments, the linker peptide comprises a sequence of SEQ ID NO: 44. In some embodiments, the antibody construct comprising the first polypeptide comprising the scFV linked to first light chain variable domain, wherein the first polypeptide comprises a sequence with at least 90% identity to SEQ ID NO: 35.

In some embodiments, the antibody construct comprising the first polypeptide comprising the scFV linked to first heavy chain variable domain, wherein the first polypeptide comprises a sequence with at least 90% identity to SEQ ID NO: 33. In some embodiments, the antibody construct comprises a first polypeptide comprising a single chain variable fragment (scFv), wherein the scFV comprises the first heavy chain variable domain linked to the first light chain variable domain. In some embodiments, the antibody construct comprising a linker peptide. that links said variable domains. In some embodiments, the linker peptide comprises a sequence of SEQ ID NO: 45. In some embodiments, the first polypeptide comprises the scFv linked to the second light chain variable domain; or the second heavy chain variable domain.

In some embodiments, the linking is by a linker peptide. In some embodiments, the linker peptide comprises a sequence of SEQ ID NO: 44. In some embodiments, the antibody construct comprising the first polypeptide comprising the scFV linked to the second light chain variable domain, wherein the first polypeptide comprises a sequence with 90% identity to SEQ ID NO: 36. In some embodiments, the antibody construct comprising the first polypeptide comprising the scFV linked to the second heavy chain variable domain, wherein the first polypeptide comprises a sequence with 90% identity to SEQ ID NO: 34. In some embodiments, the IgA heavy chain domain comprises an IgA heavy chain constant domain.

In some embodiments, the IgA heavy chain constant domain is an IgA1 constant domain or a variant thereof. In some embodiments, the IgA1 constant domain comprises at least one of an IgA1 CH2 region and an IgA1 CH3 region or variant thereof. In some embodiments, the IgA1 constant region further comprises an IgA1 CH1 region or variant thereof. In some embodiments, the IgA heavy chain constant domain is an IgA2 constant domain or variant thereof. In some embodiments, the IgA2 constant domain comprises at least one of an IgA2 CH2 region and an IgA2 CH3 region or variant thereof. In some embodiments, the IgA2 constant region further comprises an IgA2 CH1 region.

In some embodiments, the antigen is CD20, GD2, mesothelin, CD38, CD19, EGFR, HER2, PD-L1, or CD25. In some embodiments, the IgA heavy chain constant domain lacks at least one or two naturally occurring glycosylation sites, as compared to a corresponding wild type IgA. In some embodiments, said construct lacks at least two naturally occurring glycosylation sites and wherein the at least two naturally occurring glycosylation site in at least one IgA CH2 region or the IgA CH3 region. In some embodiments, the at least two naturally occurring glycosylation sites are two naturally occurring N-linked glycosylation sites. In some embodiments, the IgA heavy chain constant domain lacks at least two naturally occurring asparagine (N) amino acid residues as compared to a corresponding wild type IgA.

In some embodiments, the IgA heavy chain constant domain comprises an amino acid substitution of the at least two naturally occurring asparagine (N) amino acid residues, or a substitution of both residues. In some embodiments, the IgA heavy chain constant domain lacks at least one naturally occurring cysteine (C) amino acid residue, as compared to a corresponding wild type IgA. In some embodiments, the IgA heavy chain constant domain comprises an amino acid substitution of the at least one naturally occurring cysteine (C) amino acid residue. In some embodiments, the IgA heavy chain constant domain comprises a non-conservative amino acid substitution of the at least one naturally occurring cysteine (C) amino acid residue. In some embodiments, the IgA heavy chain constant domain comprises an amino acid deletion of the at least one naturally occurring cysteine (C) amino acid residue.

In some embodiments, the IgA heavy chain constant domain lacks at least one naturally occurring tyrosine (Y) amino acid residue, as compared to a corresponding wild type IgA. In some embodiments, the IgA heavy chain constant domain comprises an amino acid substitution of the at least one naturally occurring cysteine (Y) amino acid residue. In some embodiments, the IgA heavy chain constant domain comprises a deletion of the at least one naturally occurring cysteine (Y) amino acid residue. In some embodiments, the IgA heavy chain constant domain lacks at least one naturally occurring threonine (T) amino acid residue, as compared to a corresponding wild type IgA.

In some embodiments, the IgA heavy chain constant domain comprises an amino acid substitution of the at least one naturally occurring threonine (T) amino acid residue. In some embodiments, the IgA heavy chain constant domain comprises a deletion of the at least one naturally occurring threonine (T) amino acid residue. In some embodiments, the IgA heavy chain constant domain lacks at least one naturally occurring isoleucine (I) amino acid residue, as compared to a corresponding wild type IgA. In some embodiments, the IgA heavy chain constant domain comprises an amino acid substitution of the at least one naturally occurring isoleucine (I) amino acid residue. In some embodiments, the IgA heavy chain constant domain comprises a deletion of the at least one naturally occurring isoleucine (I) amino acid residue.

In some embodiments, the IgA heavy chain constant domain lacks at least one naturally occurring proline (P) amino acid residue, as compared to a corresponding wild type IgA. In some embodiments, the IgA heavy chain constant domain comprises an amino acid substitution of the at least one naturally occurring proline (P) amino acid residue. In some embodiments, the IgA heavy chain constant domain comprises a deletion of the at least one naturally occurring proline (P) amino acid residue.

In some embodiments, said construct exhibits a greater circulating half-life compared to a corresponding antibody construct that comprises a corresponding wild type IgA heavy chain constant region.

In some embodiments, the antibody construct comprises an attenuated CD47 binding domain that has a lower affinity for CD47 as compared to a corresponding wild-type CD47 binding domain.

In some embodiments, the antibody construct exhibits decreased aggregation compared to a corresponding antibody construct that comprises a corresponding wild type IgA heavy chain constant region. In some embodiments, the antibody construct exhibits decreased aggregation with serum proteins compared to a corresponding antibody construct that comprises a corresponding wild type IgA heavy chain constant region.

In some embodiments, said IgA heavy chain constant region comprises one or more albumin binding domains. In some embodiments, the antibody construct has a greater half-life than a corresponding antibody construct that does not comprise one or more albumin binding domains.

In some embodiments, the IgA heavy chain constant domain comprises an amino acid sequence with 90% sequence identity to a sequence selected from any one of SEQ ID NOs:37-41.

In some embodiments, the construct further comprises a hinge region. In some embodiments, the hinge region comprises an IgA hinge amino acid sequence or variant or fragment thereof.

In some embodiments, the hinge region comprises a human IgA hinge amino acid sequence or variant or fragment thereof. In some embodiments, the hinge is an IgA1 hinge or an IgA2 hinge, or variant or fragment thereof. In some embodiments, the antibody construct further comprises a light chain constant region. In some embodiments, the light chain constant region is a Kappa constant region. In some embodiments, the light chain constant region is a Lambda constant region. In some embodiments, the light chain constant region is linked to the first light chain variable domain or the first heavy chain variable domain.

In one aspect provided herein is an antibody construct that comprises: (a) an immunoglobulin A (IgA) heavy chain constant domain, (b) a CD47 binding domain; and (c) an antigen binding domain, wherein the IgA heavy chain domain specifically binds a FcαR on an immune effector cell, wherein the CD47 binding domain inhibits binding of a CD47 expressed on a target cell with a signal regulatory protein α (SIRP α) on the immune effector cell, wherein the antigen binding domain binds an antigen on the target cell, and wherein the antibody construct has a higher binding affinity for the antigen compared to the CD47.

In some embodiments, the CD47 binding domain comprises a first light chain variable domain and a first heavy chain variable domain. In some embodiments, the antigen binding domain comprises a second light chain variable domain and a second heavy chain variable domain. In some embodiments, the first light chain variable domain is of the Kappa type. In some embodiments, the second light chain variable domain is of the Lambda type.

In some embodiments, the first light chain variable domain is of the Lambda type. In some embodiments, the second light chain variable domain is of the Kappa type. In some embodiments, the first light chain variable domain is of the Kappa type comprises a variable light chain complementarity determining region 1 (CDR-L1) amino acid sequence of SEQ ID NO: 16, a variable light chain complementarity determining region 2 (CDR L2) amino acid sequence of SEQ ID NO: 17, a variable light chain complementarity determining region 3 (CDR-L3) amino acid sequence of SEQ ID NO: 18. In some embodiments, the first light chain variable domain comprises an amino acid sequence of with 90% sequence identity to SEQ ID NO: 27.

In some embodiments, the second light chain variable domain is of the Lambda type comprises a comprises a variable light chain complementarity determining region 1 (CDR-L1) amino acid sequence of SEQ ID NO: 19, a variable light chain complementarity determining region 2 (CDR L2) amino acid sequence of SEQ ID NO: 20, a variable light chain complementarity determining region 3 (CDR-L3) amino acid sequence of SEQ ID NO: 21. In some embodiments, the second light chain variable domain comprises an amino acid sequence with 90% sequence identity to SEQ ID NO: 28. In some embodiments, the first heavy chain variable domain and the second heavy chain variable domain are the same.

In some embodiments, the first heavy chain variable domain and the second heavy chain variable domain comprises a variable heavy chain complementarity determining region 1 (CDR-H1) amino acid sequence of SEQ ID NO: 7, a variable heavy chain complementarity determining region 2 (CDR-H2) amino acid sequence of SEQ ID NO: 8, a variable heavy chain complementarity determining region 3 (CDR-H3) amino acid sequence of SEQ ID NO: 9.

In some embodiments, the first heavy chain variable domain and the second heavy chain variable domain comprises an amino acid sequence with 90% sequence identity to SEQ ID NO: 24. In some embodiments, the first light chain variable domain further comprises a Kappa constant region. In some embodiments, the second light chain variable domain further comprises a Lambda constant region. In some embodiments, the first light chain variable domain further comprises a Lambda constant region. In some embodiments, the second light chain variable domain further comprises a Kappa constant region. In some embodiments, the Kappa constant region comprises an amino acid sequence with 95% sequence identity to SEQ ID NO: 42. In some embodiments, the Lambda constant region comprises an amino acid sequence with 95% sequence identity to SEQ ID NO: 43. In some embodiments, the IgA heavy chain constant domain comprises at least one of an IgA CH2 region and an IgA CH3 region or variant thereof.

In some embodiments, the IgA heavy chain constant domain further comprises an IgA CH1 region or variant thereof. In some embodiments, the CD47 binding domain is linked to the IgA constant domain by the IgA CH1 domain. In some embodiments, the antigen binding domain is linked to the IgA constant domain by the IgA CH1 domain. In some embodiments, the IgA heavy chain constant domain is an IgA1 constant region or variant thereof. In some embodiments, the IgA1 constant region comprises an IgA1 CH2 region and an IgA1 CH3 region.

In some embodiments, the IgA1 constant region further comprises an IgA1 CH1 region. In some embodiments, the IgA heavy chain constant domain is an IgA2 constant region or variant thereof. In some embodiments, the IgA2 constant region comprises an IgA2 CH2 region and an IgA2 CH3 region. In some embodiments, the IgA2 constant region further comprises an IgA2 CH1 region. In some embodiments, the antigen is CD20, GD2, mesothelin, CD38, CD19, EGFR, HER2, PD-L1, or CD25.

In some embodiments, the IgA heavy chain constant domain lacks one, two or three naturally occurring glycosylation sites, as compared to a corresponding wild type IgA. In some embodiments, said sites are in at least one of the IgA CH2 region or the IgA CH3 region. In some embodiments, said construct lacking two naturally occurring N-linked glycosylation sites. In some embodiments, the IgA heavy chain constant domain lacks a naturally occurring asparagine (N) amino acid residues as compared to a corresponding wild type IgA.

In some embodiments, the IgA heavy chain constant domain comprises an amino acid substitution of at least one naturally occurring asparagine (N) amino acid residues, or a substitution of at least two naturally occurring asparagine (N) amino acid residues. In some embodiments, the IgA heavy chain constant domain lacks at least one naturally occurring cysteine (C) amino acid residue, as compared to a corresponding wild type IgA. In some embodiments, the IgA heavy chain constant domain comprises an amino acid substitution of the at least one naturally occurring cysteine (C) amino acid residue. In some embodiments, the IgA heavy chain constant domain comprises a non-conservative amino acid substitution of the at least one naturally occurring cysteine (C) amino acid residue.

In some embodiments, the IgA heavy chain constant domain comprises an amino acid deletion of the at least one naturally occurring cysteine (C) amino acid residue. In some embodiments, the IgA heavy chain constant domain lacks at least one naturally occurring tyrosine (Y) amino acid residue, as compared to a corresponding wild type IgA. In some embodiments, the IgA heavy chain constant domain comprises an amino acid substitution of the at least one naturally occurring cysteine (Y) amino acid residue. In some embodiments, the IgA heavy chain constant domain comprises a deletion of the at least one naturally occurring cysteine (Y) amino acid residue.

In some embodiments, the IgA heavy chain constant domain lacks at least one naturally occurring threonine (T) amino acid residue, as compared to a corresponding wild type IgA. In some embodiments, the IgA heavy chain constant domain comprises an amino acid substitution of the at least one naturally occurring threonine (T) amino acid residue. In some embodiments, the IgA heavy chain constant domain comprises a deletion of the at least one naturally occurring threonine (T) amino acid residue. In some embodiments, the IgA heavy chain constant domain lacks at least one naturally occurring isoleucine (I) amino acid residue, as compared to a corresponding wild type IgA.

In some embodiments, the IgA heavy chain constant domain comprises an amino acid substitution of the at least one naturally occurring isoleucine (I) amino acid residue. In some embodiments, the IgA heavy chain constant region comprises a deletion of the at least one naturally occurring isoleucine (I) amino acid residue. In some embodiments, the IgA heavy chain constant domain lacks at least one naturally occurring proline (P) amino acid residue, as compared to a corresponding wild type IgA. In some embodiments, the IgA heavy chain constant region comprises an amino acid substitution of the at least one naturally occurring proline (P) amino acid residue.

In some embodiments, the IgA heavy chain constant domain comprises a deletion of the at least one naturally occurring proline (P) amino acid residue. In some embodiments, the antibody construct exhibits a greater circulating half-life compared to a corresponding antibody construct that comprises a corresponding wild type IgA heavy chain constant region. In some embodiments, the antibody construct exhibits a greater half-life compared to a corresponding antibody construct that comprises a corresponding wild type IgA heavy chain constant region. In some embodiments, the antibody construct exhibits decreased aggregation compared to a corresponding antibody construct that comprises a corresponding wild type IgA heavy chain constant region. In some embodiments, the antibody construct exhibits decreased aggregation with serum proteins compared to a corresponding antibody construct that comprises a corresponding wild type IgA heavy chain constant region.

In some embodiments, said IgA heavy chain constant region comprises one or more albumin binding domains. In some embodiments, the antibody construct has a greater half-life than a corresponding antibody construct that does not comprise one or more albumin binding domains. In some embodiments, the IgA heavy chain constant region further comprises a hinge region. In some embodiments, wherein the hinge region comprises an IgA hinge amino acid sequence or variant or fragment thereof.

In some embodiments, the hinge region comprises a human IgA hinge amino acid sequence or variant or fragment thereof. In some embodiments, the hinge is an IgA1 hinge or an IgA2 hinge, or variant or fragment thereof. In some embodiments, the antibody construct further comprises an immunoadhesion molecule, an imaging agent, a therapeutic agent, or a cytotoxic agent. In some embodiments, the imaging agent is a radiolabel, an enzyme, a fluorescent label, a luminescent label, a bioluminescent label, a magnetic label, or biotin. In some embodiments, said therapeutic or cytotoxic agent is an anti-metabolite, an alkylating agent, an antibiotic, a growth factor, a cytokine, an anti-angiogenic agent, an anti-mitotic agent, an anthracycline, a toxin, or an apoptotic agent. In some embodiments, the immune effector cell is a neutrophil.

In some embodiments, the antibody construct only inhibits the signal regulatory protein α (SIRP α) function on the immune effector cell when also the antigen binding domain is bound to the target cell that expresses CD47. In some embodiments, the antibody construct is bispecific. In some embodiments, the CD47 binding domain is a Fab, Fab′, Fab′-SH, Fv, scFv, F(ab′)2, a diabody, a linear antibody, a single domain antibodies (sdAb), or a camelid VHH domain. In some embodiments, the antigen binding domain is a Fab, Fab′, Fab′-SH, Fv, scFv, F(ab′)2, a diabody, a linear antibody, a single domain antibodies (sdAb), or a camelid VHH domain. In some embodiments, the IgA heavy chain domain is a heterodimer of two IgA heavy chain constant domain. In some embodiments, the heterodimerization is by knobs-into-holes coupling, salt bridges/electrostatic complementarity coupling, CrossMab coupling, strand-exchange engineered domain technology, or a combination thereof. In some embodiments, the antigen binding domain binds an antigen of a cancer cell or a pathogen. In some embodiments, the pathogen is a microbe, microorganism, or a virus.

In one aspect provided herein is, an antibody construct comprising; (a) an immunoglobulin A (IgA) heavy chain domain, (b) a CD47 binding domain; and (c) an antigen binding domain; wherein the IgA heavy chain domain specifically binds a FcαR on an immune effector cell, wherein the CD47 binding domain inhibits binding of a CD47 expressed on a target cell with signal regulatory protein α (SIRP α) on the immune effector cell, wherein the antigen binding domain binds an antigen on the target cell, wherein the antigen is CD20, GD2, mesothelin, CD38, CD19, EGFR, HER2, PD-L1, or CD25, and wherein the antibody construct has a higher binding affinity for the antigen compared to the CD47.

In one aspect provided herein is a multispecific immune effector cell engager molecule comprising; (a) an immunoglobulin A (IgA) heavy chain domain; (b) a CD47 binding domain; and (c) an antigen binding domain, wherein the IgA heavy chain domain specifically binds a FcαR on an immune effector cell, wherein the CD47 binding domain inhibits binding of a CD47 expressed on a target cell with a signal regulatory protein α (SIRP α) on the immune effector cell, wherein the antigen binding domain binds an antigen on the target cell, and wherein the antibody construct has a higher binding affinity for the antigen compared to the CD47.

In one aspect provided herein is a pharmaceutical composition comprising any one of the antibody construct above, and a pharmaceutically acceptable carrier, adjuvant or diluent. In some embodiments, the pharmaceutical composition further comprising at least one additional therapeutic agent. In some embodiments, said additional therapeutic agent is an imaging agent, a cytotoxic agent, an angiogenesis inhibitor, a kinase inhibitor, a co-stimulation molecule blocker, an adhesion molecule blocker, an anti-cytokine antibody or functional fragment thereof, methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog thereof, a cytokine, or a cytokine antagonist.

In one aspect provided herein is an isolated nucleic acid encoding the antibody construct of any one of aspects above.

In one aspect provided herein is a vector comprising the isolated nucleic acid described above.

In one aspect provided herein is a n in vitro cell comprising the isolated nucleic acid of disclosed above.

In one aspect provided herein is an in vitro cell expressing the antibody construct of any one of aspects above.

In one aspect provided herein is a method of treating a subject in need thereof, comprising administering to the subject an effective amount of a composition comprising an antibody construct, wherein the antibody construct comprises, (a) an immunoglobulin A (IgA) heavy chain domain, (b) a CD47 binding domain; and (c) an antigen binding domain, wherein the IgA heavy chain domain specifically binds a FcαR on an immune effector cell, wherein the CD47 binding domain inhibits binding of a CD47 expressed on a target cell with a signal regulatory protein α (SIRP α) on the immune effector cell, wherein the antigen binding domain binds an antigen on the target cell, and wherein the antibody construct has a higher binding affinity for the antigen compared to the CD47.

In some embodiments, the inhibition of the CD47 binding with the SIRP α increases phagocytosis and clearance of the target cell. In some embodiments, the composition is administered orally, intralesionally, by intravenous therapy or by subcutaneous, intramuscular, intraarterial, intravenous, intracavitary, intracranial, or intraperitoneal injection. In some embodiments, the composition is administered daily, weekly, biweekly, monthly, every two months, once every three months, once every 6 months, or once every 12 months. In some embodiments, the subject has cancer. In some embodiments, the cancer is selected from selected from the group consisting of acute lymphoblastic leukemia, acute myelogenous leukemia, biliary cancer, B-cell leukemia, B-cell lymphoma, biliary cancer, bone cancer, brain cancer, breast cancer, triple negative breast cancer, cervical cancer, Burkitt lymphoma, chronic lymphocytic leukemia, chronic myelogenous leukemia, colorectal cancer, endometrial cancer, esophageal cancer, gall bladder cancer, gastric cancer, gastrointestinal tract cancer, glioma, hairy cell leukemia, head and neck cancer, Hodgkin's lymphoma, liver cancer, lung cancer, medullary thyroid cancer, melanoma, multiple myeloma, ovarian cancer, non-Hodgkin's lymphoma, pancreatic cancer, prostate cancer, pulmonary tract cancer, renal cancer, sarcoma, skin cancer, testicular cancer, urothelial cancer, and urinary bladder cancer.

In some embodiments, the subject is human. In some embodiments, the method comprises administering an effective amount of at least one additional therapeutic agent. In some embodiments, the additional therapeutic agent is an imaging agent, a chemotherapeutic agent, a kinase inhibitor, a co-stimulation molecule blocker, an adhesion molecule blocker, a second antibody or antigen-binding fragment thereof, a drug, a toxin, an enzyme, a cytotoxic agent, an anti-angiogenic agent, a pro-apoptotic agent, an antibiotic, a hormone, an immunomodulator, a cytokine, a chemokine, an antisense oligonucleotide, a small interfering RNA (siRNA), methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, or an antipsychotic.

In one aspect provided herein is a method of inducing a neutrophil mediated immune response to a target cell comprising: contacting the target cell with an effective amount of an antibody construct, wherein the antibody construct comprises; (a) an immunoglobulin A (IgA) heavy chain domain; (b) a CD47 binding domain; and (c) an antigen binding domain; wherein the IgA heavy chain domain specifically binds a FcαR on a neutrophil, wherein the CD47 binding domain inhibits binding of a CD47 expressed on a target cell with a signal regulatory protein α (SIRP α) on the neutrophil, wherein the antigen binding domain binds an antigen on the target cell, and wherein the antibody construct has a higher binding affinity for the antigen compared to the CD47, thereby inducing the neutrophil mediated immune response.

In some embodiments, the neutrophil mediated immune response comprises phagocytosis of the target cell or lyses of the target cell. In some embodiments, the antigen presenting cell is a cancer cell, or a viral cell. In some embodiments, the cancer cell is a lymphocyte.

In some embodiments, the CD47 binding domain comprises the first light chain variable domain comprising a variable light chain complementarity determining region 1 (CDR-L1), a variable light chain complementarity determining region 2 (CDR L2), and a variable light chain complementarity determining region 3 (CDR-L3), wherein the CDR-L1, CDR-L2 and CDR-L3 comprises an amino acid sequence selected from Table A.

In some embodiments, the CD47 binding domain comprises the first heavy chain variable domain comprising a variable light chain complementarity determining region 1 (CDR-H1), a variable light chain complementarity determining region 2 (CDR H2), and a variable light chain complementarity determining region 3 (CDR-H3), wherein the CDR-H1, CDR-H2 and CDR-H3 comprises an amino acid sequence selected from Table A.

In some embodiments, the antigen binding domain comprises the second light chain variable domain comprising a variable light chain complementarity determining region 1 (CDR-L1), a variable light chain complementarity determining region 2 (CDR L2), and a variable light chain complementarity determining region 3 (CDR-L3), wherein the CDR-L1, CDR-L2 and CDR-L3 comprises an amino acid sequence selected from Table B.

In some embodiments, the antigen binding domain comprises the second heavy chain variable domain comprising a variable light chain complementarity determining region 1 (CDR-H1), a variable light chain complementarity determining region 2 (CDR H2), and a variable light chain complementarity determining region 3 (CDR-H3), wherein the CDR-H1, CDR-H2 and CDR-H3 comprises an amino acid sequence selected from Table B.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1E shows exemplary antibody constructs described herein. Preparation of illustrative constructs is explained in the Examples. FIG. 1A shows exemplary IgA constant domain scaffold based bivalent dimer construct (in exemplary constructs described herein, the IgA heavy chain scaffold of an IgA1, IgA2 or a modified IgA2 with reduced glycosylation and/or better expression profile (also referred to as IgA2.0 or IgA3.0) is exemplified), with specificity determined by VH and kappa VL regions that are connected to the IgA scaffold, wherein said construct can bind CD47 and an exemplary antigen on an antigen presenting cell (in an exemplary case, CD20). FIG. 1B shows a bivalent heterodimer consisting of a common heavy chain, and both a kappa and a lambda light chain. The specificity is only determined by VL regions of both the kappa and lambda regions, wherein one such region specifically binds CD47 and the other specifically binds an exemplary antigen on an antigen presenting cell (in an exemplary case, CD20). FIG. 1C shows DVD-IgA2 based construct, a tetravalent homodimer. Specificity is determined by the combination of two VH/VL pairs, an additional VH has been linked to the original VH, and an additional VL has been linked to the original VL, which are linked by peptide linkers to the pre-existing VH and VL. FIG. 1D shows DVD-IgA3.0-scFv_LC, a tetravalent homodimer, wherein IgA3.0 is an IgA optimized for glycosylation and manufacturability. Specificity is determined by the combination of an scFv which is linked to the original VL. FIG. 1E shows DVD-IgA3.0-scFv_HC, a tetravalent homodimer. Specificity is determined by the combination of an scFv linked to the original VH. In embodiments described herein the construct specifically binds CD47 and antigen on an antigen presenting cell (in an exemplary cases, CD20, HER2, GD2, mesothelin, EGFR etc). In antibody construct designs described herein in any of FIGS. 1A-1E, the antigen binding region and the CD47 binding region can be derived from commercially available or otherwise publicly known antibodies. In certain embodiments, the antibody construct is designed to have a CD47 binding domain or region that has a lower binding affinity for CD47 than the antigen binding domain or region in the construct.

FIGS. 2A-2K demonstrate an optimal heavy chain:light chain ratios needed for expression of an IgA antibody construct described herein in HEK293-Freestyle (HEK293F) cells. X axis shows ratio of heavy chain encoding DNA (HC) and light chain encoding DNA (KLC) in combination with pAdvantage-pAdv (Promega) in a total concentration of 1 ug/mL in a volume of 2 mL. The amount of pAdv DNA is always equal to HC DNA. Y axis shows protein concentration measured for individual antibody construct. Protein concentrations were determined for each of the bispecific antibodies in table 1 and table 2. Protein concentrations are determined by kappa-IgA ELISA for DVD-IgA bispecific antibody #1 (FIG. 2A) and DVD-IgA bispecific antibody #2 (FIG. 2B), DVD-IgA-scFv_LC bispecific antibody #5 (FIG. 2C), DVD-IgA-scFv_LC bispecific antibody #6 (FIG. 2D), DVD-IgA-scFv_HC bispecific antibody #3 (FIG. 2E), DVD-IgA-scFv_HC bispecific antibody #4 (FIG. 2F). Protein concentrations measured by kappa/lambda-IgA ELISA for kappa-kappa IgA (FIGS. 2G-2H) and lambda-lambda IgA (FIGS. 2I-2J). OD415 values for the kappa-lambda IgA, as no concentration could be measured due to lack of a standard (FIG. 2K). Methods are described in the Examples section 3.

FIGS. 3A-3B show the purification of an illustrative recombinant IgA antibody construct described herein. FIG. 3A illustrates affinity purification elution profile of an exemplary bispecific DVD-IgA molecule. FIG. 3B shows purity of bispecific antibodies #1 and #5 on non-reducing SDS-PAGE gel at expected molecular weights. The gel shows low level of non-associated kappa light chains (35 kDa for #1; 50 kDa for #5). Methods are described in the Examples section 3.

FIG. 4 shows measured concentrations of six exemplary antibody constructs (DVD-IgA) described herein (e.g., listed in Table 1). All six bispecific IgA molecules were produced in HEK293F cells and the concentrations were measured by ELISA in the supernatant.

FIGS. 5A-5D show the gating strategy in flow cytometric analysis and identification of cells expressing IgA antibody constructs. FIG. 5A shows selection of a live cellular population based on cellular size and shape. FIG. 5B shows further selection of single cells from the live cellular population. FIG. 5C shows the gating strategy for control IgA-negative cells after staining with an anti-IgA PE-labeled antibody. FIG. 5D shows analysis and selection of IgA positive cells by staining with an anti-IgA PE-labeled antibody.

FIGS. 6A-6B demonstrate binding analysis of antibody constructs described herein (e.g., Table 1 and Table 2) by flow cytometry. FIG. 6A shows characterization SKBR3 cell lines used. Both SKBR3 WT and SKBR3-CD20 cells have been stained with IgA antibodies directed against CD20 (Obi=complementarity region of Obinituzumab) or CD47 (clone 2.3D11). Unstained cells and secondary antibody only are controls. FIG. 6B shows binding of bispecific antibodies to SKBR3 WT and SKBR3-CD20 cells. X-axis left panel in FIG. 6B identifies the antibody, e.g., anti-CD47 antibody 2.3D11 alone, antibody #1, antibody #2, antibody #3, antibody #4, antibody #5, and antibody #6. X-axis right panel in FIG. 6B identifies the antibody, e.g., anti-CD20 Obi antibody alone, antibody #1, antibody #2, antibody #3, antibody #4, antibody #5, and antibody #6. Supernatants containing individual bispecific antibodies, #1-6, have been tested both on SKBR3 WT (left panel) and SKBR3-CD20 (right panel) cells in the absence (black) or presence of a CD47 blocking antibody (grey). Bispecific antibodies #2, #3, and #5 show an effect of CD47 blocking, illustrating CD20 binding is enhanced by additional CD47 binding.

FIGS. 7A-7B show antibody-dependent cell-mediated cytotoxicity (ADCC) analysis of bispecific antibodies. SKBR3-CD20 cells have been used as target cells in an PMN ADCC assay. The antibodies IgA3.0-Obi and IgA3.0-2.3D11 have been used either alone or in combination, and bispecific IgA antibody #2 has been tested in parallel. The combination (Obi+2.3D11) and the bispecific antibodies have been pre-blocked using mIgG1 CD47 PerCP-Cy5.5 antibody. Upon pre-blocking of CD47, there is a reduction in killing observed for the combination and the bispecific. The functional effect by bispecific blocking of CD47 enhances CD20 dependent ADCC. FIG. 7A demonstrates Obi-IgA and 2.3D11-IgA dependent ADCC activity with PMN using either SKBR3 (CD20−/−) WT (top panel) or SKBR3 CD20(+) (bottom panel) as target cells. SKBR3 WT and CD20 cells do not show ADCC with IgG1. The SKBR3 WT does not show specific lysis with Obi-IgA only, whereas 2.3D11 induces ADCC. In the SKBR3-CD20 (+) cells, Obi-IgA induced further ADCC in combination with 2.3D11-IgA, which partially restored upon pre-blockage of CD47 using mIgG1-anti-CD47 PerCP-Cy5.5. On the x-axis antibody concentrations are given in ug/mL. FIG. 7B demonstrates SKBR3-CD20(+) cells in an PMN ADCC assay to test ADCC activity of the bispecific antibody #2, DVD-IgA Obi-2.3D11. As a control, IgA3.0-Obi and IgA3.0-2.3D11 have been used either alone or in combination. The combination (Obi+2.3D11) demonstrated the highest ADCC which could be efficiently blocked by pre-incubating the SKBR3-CD20 (+) cells with mIgG1 CD47 PerCP-Cy5.5 antibody. Bispecific antibody #2 showed similar killing efficiency as compared to the combination. This effect could only be partially reduced by a pre-block with mIgG1 antiCD47 PerCP-Cy5.5. Thus, the functional effect by blocking CD47 in the same molecule, the bispecific IgA, greatly enhances CD20 dependent ADCC.

FIGS. 8A-8D show analysis of CD47 binding by antibody constructs.

FIG. 8A shows gating strategy of the erythrocytes. CD235a+ cells have been selected, and gated on secondary IgA antibody only as negative gate. CD47 is highly positive on erythrocytes. FIG. 8B shows Gating strategy of the platelets. CD61+ cells have been selected, and gated on secondary IgA antibody only as negative gate. CD47 is highly positive on platelets. FIGS. 8C-8D show flow cytometric analysis of binding of antibody constructs described herein to Erythrocytes (FIG. 8C) and Platelets (FIG. 8D). FIG. 8C shows analysis of CD47 binding on erythrocytes demonstrates low level of CD47 binding on erythrocytes by bispecific antibody #4 (Obi-scFv:2.3D11_HC). No binding to erythrocytes was observed for all other bispecific. FIG. 8D demonstrates no platelet binding is observed of the bispecific antibodies.

FIG. 9 shows an illustration describing the mechanism of action of the antibody constructs described herein. Antibody construct of the disclosure comprises an antigen binding domain, CD47 binding domain and the IgA heavy chain constant domain. Antibody construct binds an antigen of a target cell via the antigen binding domain and CD47 on the same target cell by the CD47 binding domain and the Fc receptor on an immune effector cell (e.g., a neutrophil) by the IgA heavy chain constant domain. The binding to the CD47 inhibits the interaction of SIRPα on the immune effector cell with the CD47 on the target cell, thereby inhibiting the “don't eat me signal” and inducing immune effector cell response to the target cell.

### DETAILED DESCRIPTION OF THE DISCLOSURE

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, an “antigen on an antigen presenting cell” refers to an antigenic substance associated with an antigen presenting, and that can trigger an immune response in a host. For instance, an antigen is any antigenic substance produced or overexpressed in a pathogenic cell, for instance a cancer cell. Antigens are proteins, peptides or polysaccharides. Antigen presenting cells as described herein, are cells that present antigens, for instance in the form of peptides on histocompatibility molecules. A cancer cell, for instance a tumor cell that presents a tumor antigen is an exemplary antigen presenting cell. A tumor antigen is an exemplary antigen in constructs described herein, wherein said tumor antigen is produced in a tumor cells. It may, for example, trigger an immune response in the host. Alternatively, for purposes of this disclosure, tumor antigens may be proteins that are expressed by both healthy and tumor cells but because they identify a certain tumor type, or are overexpressed in a certain tumor type, are a suitable therapeutic target. In embodiments, the tumor antigen is CD20, GD2, CD38, CD19, EGFR, HER2, PD-L1, CD25, CD33, BCMA, CD44, α-Folate receptor, CAIX, CD30, ROR1, CEA, EGP-2, EGP-40, HER3, Folate-binding Protein, GD3, IL-13R-a2, KDR, EDB-F, mesothelin, CD22, EGFR, MUC-1, MAGE-A1, MUC16, h5T4, PSMA, TAG-72, EGFRvIII, CD123 or VEGF-R2. In one embodiment, the tumor antigen is EGFRvIII, which is a target for therapeutics treating myeloid malignancies, for example, glioblastoma or glioblastoma multiforme (GBM). In another embodiment, the tumor antigen is CD20. In yet another embodiment, the tumor antigen is GD2. In yet another embodiment, the tumor antigen is mesothelin.

As used herein, the term “antibody” refers to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, humanized, heteroconjugate, bispecific, diabody, triabody, or tetrabody. The antigen binding fragment can include, for example, Fab′, F(ab′)2, Fab, Fv, rlgG, scFv, hcAbs (heavy chain antibodies), a single domain antibody, VHH, VNAR sdAbs, or nanobody. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding. In constructs described herein, antibodies comprise an IgA heavy chain domain or region that forms a scaffold for attachment of CD47 binding and/or antigen binding domains. An IgA constant region domain is based on an IgA1 or IgA2 constant region domain that in some cases is further modified for improved glycosylation profile, improved manufacturability, ease of heterodimer formation or tailored/selective Fc receptor binding.

As used herein a “recombinant antibody” is an antibody that comprises an amino acid sequence derived from two different species or, or two different sources, and includes synthetic molecules. By way of non-limiting example, an antibody that comprises a non-human CDR and a human variable region framework or constant or Fc region, an antibody with binding domains from two different monoclonal antibodies, or an antibody comprising a mutation of one or more amino acid residues to increase or decrease biological activity or binding of a part of the antibody. In certain embodiments, recombinant antibodies are produced from a recombinant DNA molecule or synthesized. In certain embodiments, the antibodies described herein are a polypeptide(s) encoded by one or more polynucleotides.

As used herein, “recognize” or “bind” or “selective for” refers to the association or binding between an antigen binding domain and an antigen. As used herein, an “antigen” refers to an antigenic substance that can trigger an immune response in a host. An antigenic substance can be a molecule, such as a costimulatory molecule that can trigger an immune response in a host.

As used herein, an “antibody construct” refers to a construct that contains an antigen binding domain and an Fc domain.

As used herein, a “binding domain” refers to an antibody or non-antibody domain.

As used herein, an “antigen binding domain” refers to a binding domain from an antibody or from a non-antibody that can bind to an antigen. An antigen binding domain can be a tumor antigen binding domain or a binding domain that can bind to an antigen (such as a molecule) on an antigen presenting cell. Antigen binding domains can be numbered when there is more than one antigen binding domain in a given conjugate or antibody construct (e.g., first antigen binding domain, second antigen binding domain, third antigen binding domain, etc.). Different antigen binding domains in the same conjugate or construct can target the same antigen or different antigens (e.g., first antigen binding domain that can bind to a tumor antigen, second antigen binding domain that can bind to a molecule on an antigen presenting cell (APC antigen), and third antigen binding domain that can bind to an APC antigen).

As used herein, an “antibody antigen binding domain” refers to a binding domain from an antibody that can bind to an antigen.

As used herein, an “Fc domain” refers to an Fc domain from an antibody or from a non-antibody that can bind to an Fc receptor. As used herein, an “Fc domain” and an “Fc comprising domain” can be used interchangeably.

As used herein, a “target binding domain” refers to a construct that contains an antigen binding domain from an antibody or from a non-antibody that can bind to an antigen.

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gin); glycine (G, Gly); histidine (H, His); isoleucine (I, He); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

The phrase “pharmaceutically acceptable” is employed herein to refer to those compounds, materials, compositions, and/or dosage forms which are, within the scope of sound medical judgment, suitable for use in contact with the tissues of subjects for instance, human beings and animals, without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An antigen can elicit an immune response. An antigen can be a protein, polysaccharide, lipid, or glycolipid, which can be recognized by an immune cell, such as a T cell or a B cell. Exposure of immune cells to one or more of these antigens can elicit a rapid cell division and differentiation response resulting in the formation of clones of the exposed T cells and B cells. B cells can differentiate into plasma cells which in turn can produce antibodies which selectively bind to the antigens.

The terms “cancer,” “tumor,” and “proliferative disease” relate to the physiological condition in mammals characterized by deregulated cell growth. Cancer is a class of diseases in which a group of cells display uncontrolled growth or unwanted growth. Cancer cells can also spread to other locations, which can lead to the formation of metastases. Spreading of cancer cells in the body can, for example, occur via lymph or blood. Uncontrolled growth, intrusion and metastasis formation are also termed malignant properties of cancers. These malignant properties differentiate cancers from benign tumors, which typically do not invade or metastasize.

“Antigen recognition moiety” or “antibody recognition domain” refers to a molecule or portion of a molecule that specifically binds to an antigen. In one embodiment, the antigen recognition moiety is an antibody, antibody like molecule or fragment thereof and the antigen is a tumor antigen or an infectious disease antigen.

The terms “fragment of an antibody,” “antibody fragment,” “functional fragment of an antibody,” “antigen binding domain” or their grammatical equivalents are used interchangeably herein to mean one or more fragments or portions of an antibody that retain the ability to specifically bind to an antigen (see, generally, Holliger et al., Nat. Biotech., 23(9):1126-1129 (2005)). The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Pat. No. 8,603,950. Other antibody fragments can include variable fragments of heavy chain antibodies (VHH).

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge may be maintained; glutamic acid for aspartic acid and vice versa such that a negative charge may be maintained; serine for threonine such that a free —OH can be maintained; and glutamine for asparagine such that a free —NH₂ can be maintained. Alternatively or additionally, the therapeutic agents can comprise the amino acid sequence of the reference protein with at least one non-conservative amino acid substitution.

The terms “non-conservative mutation” or “non-conservative amino acid substitution” involve amino acid substitutions between different groups, for example, lysine for tryptophan, or phenylalanine for serine, etc. In this case, it is preferable for the non-conservative amino acid substitution to not interfere with, or inhibit the biological activity of the therapeutic agent. The non-conservative amino acid substitution may enhance the biological activity of the therapeutic agent, such that the biological activity of the therapeutic agent is increased as compared to the wild type therapeutic agent.

### IgA Antibodies

Immunoglobulin A (IgA) is known for its anti-microbial role and is abundantly present in its dimeric form at mucosal sites. As a monomer, it is the second most prevalent antibody present in serum. IgA comprises two subclasses, IgA1 and IgA2, which bind with similar affinity to the myeloid IgA receptor (FcαRI, CD89). In some embodiments, IgA antibodies have a superior ability to recruit neutrophils for antibody-dependent cell-mediated cytotoxicity (ADCC). In some embodiments, IgA antibodies require lower effector:target (E:T) ratios. In some embodiments, IgA antibodies lower tumor-opsonizing antibody concentrations compared to other types of antibodies (e.g., IgG). In general, cancer therapeutic antibodies act by a combination of both direct as well as indirect immune-mediated effects. These effects include cytotoxicity induced by complement activation, antibody-dependent cellular phagocytosis (ADCP) and antibody-dependent cellular cytotoxicity (ADCC). ADCC can be mediated through activation of different Fc-receptor expressing cells, including natural killer (NK) cells, macrophages and neutrophils. Of these Fc-receptor expressing effector cells, macrophages and neutrophils express the FcαRI needed to bind IgA antibodies, and hence can kill tumor cells by ADCP or ADCC.

In some embodiments, IgA antibodies trigger immune cell mediated (for instance neutrophil-mediated) phagocytosis or trogocytosis of pathogenic cells such as tumor cells following IgA antibody-immune cell engagement. This mechanism of killing tumor cells is mediated mainly by interacting with the Fc receptor for IgA (FcαRI; CD89), which is the best characterized IgA receptor. FcαRI is expressed on monocytes, macrophages, granulocytes, subsets of dendritic cells, and Kupffer cells and binds both monomeric and dimeric IgA isoforms with median affinity. Binding of IgA to FcαRI mediates effector functions such as phagocytosis, oxidative burst, cytokine release, antigen presentation, and ADCC. In humans, two IgA isotypes, IgA1 and IgA2, and three allotypes, IgA2m(1), IgA2m(2) and IgA2n, have been distinguished. In some embodiments, IgA antibodies trigger polymorphonuclear cell (PMN)-mediated ADCC more efficiently than IgG antibodies.

IgA has two subclasses (IgA1 and IgA2) and can be produced as a monomeric as well as a dimeric form and secretory form. In some embodiments, the IgA antibody can be monomeric. In some embodiments, the IgA antibody can comprise one or more IgA1 amino acid sequences. In some embodiments, the IgA antibody can comprise one or more IgA2 amino acid sequences. In some embodiments, the IgA antibody can comprise one or more IgA1 amino acid sequences and one or more IgA2 amino acid sequences.

In some embodiments, an antibody construct comprises a constant region domain from an IgA antibody which is an IgA1 or an Ig! Antibody. In some cases, an IgA2 antibody is an IgA2 antibody of allotype: IgA2m(1), IgA2(m)2, or IgA2n. In some embodiments, the IgA2m(1) antibody is a Caucasian IgA2m(1) antibody. In some embodiments, the IgA2m(2) antibody is an African IgA2m(2) antibody or an Asian IgA2m(2) antibody. In some embodiments is an IgA2 antibody that has been modified for improved manufacturability, favorable glycosylation, improved solubility or improved activity for instance selective Fc receptor binding. Exemplary IgA2 based constant regions or use in antibody constructs described herein, with such improvements such as IgA2.0 and IgA3.0 are provided herein. Further such modifications based on the knowledge of the skilled artisan are within the purview of constructs described herein.

In some embodiments, an antibody construct described herein comprises a heavy chain constant region comprising at least 60%, 70%, 75%, 80%, 85%, 90%, 95%, or 99% IgA amino acids. In some embodiments, the IgA antibody comprises a light chain constant region comprising at least 50, 60%, 70%, 75%, 80%, 85%, 90%, 95%, or 99% IgA amino acids.

In some embodiments, an antibody construct described herein comprises a heavy chain constant region comprising one or more of an IgA CH3 region, an IgA CH2, or an IgA CH1 region, or any combination thereof. In some embodiments, an antibody construct described herein comprises a light chain region domain comprising an IgA CH1 region. In some embodiments an antibody construct described herein comprises a heavy chain constant region comprising one or more amino acid of an IgG CH3 region, an IgG CH2, or an IgG CH1 region, or any combination thereof. In some embodiments, an antibody construct described herein comprises a heavy chain constant region comprising an IgA CH3 region, an IgA CH2, and an IgA CH1 region, or any combination thereof. In some embodiments, an antibody construct described herein comprises a heavy chain constant region comprising an IgA CH3 region, an IgA CH2, and an IgA or IgG CH1 region, or any combination thereof.

In some embodiments, an antibody construct described herein comprises an IgG variable region, for instance a light chain variable region that is fused or connected to an IgA constant region optionally by use of a linker. In some embodiments, an antibody construct described herein comprises an IgG heavy chain variable region. In some embodiments, an antibody construct described herein comprises an IgG light chain variable region and an IgG heavy chain variable region.

In some embodiments, an antibody construct described herein can comprise one or more domains from a humanized antibody. In some embodiments, the an antibody construct described herein can comprise one or more domains from a chimeric, murine, camelid or shark antibody. In some embodiments, an antibody construct described herein can comprise domains exclusively from a human antibody.

In some embodiments, an antibody construct described herein can be a bi-specific antibody, or multi-specific antibody construct. In some embodiments, an antibody construct described herein can be a tri-specific antibody. In some embodiments, an antibody construct described herein can be a multi-specific antibody.

In some embodiments, an antibody construct described herein can be a bispecific antibody. In some embodiments, an antibody construct described herein co-engages CD47 and one or more antigens at the cell surface. In some examples, the binding of an antibody construct described herein to two different antigens is sequential. For example, the binding of the construct to the first antigen occurs first and thereby restricts the space explored by the second antibody arm. Consequentially, there can be a significant increase in local concentration of the second antigen, which can facilitate the binding of the second antibody arm. In some cases, such a first antigen is an antigen selectively expressed or overexpressed on an antigen presenting cell, and the second antigen is CD47 which is also expressed on the antigen presenting cell. In some cases the construct binds the second antigen with less binding strength than the first antigen, thereby allowing the binding to the first antigen to dictate cell specificity.

In some embodiments, the construct can comprise at least a portion of the Fc domain of an IgA or variant thereof. In some embodiments, an antibody construct described herein comprises a heavy chain constant region comprising a CH3, CH2, and CH1 domain. In some embodiments, an antibody construct described herein comprises a light chain constant region comprising a CH1.

In some embodiments, the construct induces complement-dependent cytotoxicity (CDC). In some embodiments, the construct induces polymorphonuclear neutrophil (PMN)-mediated tumor cell lysis. In some embodiments, the IgA antibody induces programmed cell death (PCD) via a caspase-independent pathway. In some embodiments, the antibody construct induces antibody-dependent cell-mediated cytotoxicity (ADCC). In some embodiments, the IgA antibody induces antibody-dependent cell-mediated cytotoxicity (ADCC) mediated by neutrophils.

In some embodiments, an antibody construct described herein can have a superior ability to recruit neutrophils for antibody-dependent cell-mediated cytotoxicity (ADCC) compared to a corresponding IgG antibody. In some embodiments, the IgA antibody can require lower effector:target (E:T) ratios. In some embodiments, an antibody construct described herein can require lower tumor-opsonizing antibody concentrations compared to other types of antibodies (e.g., IgG). In some embodiments, an antibody construct described herein can trigger neutrophil-mediated phagocytosis or trogocytosis of tumor cells following IgA antibody-neutrophil engagement. This mechanism of killing tumor cells is mediated mainly by interacting with the Fc receptor for IgA (FcαRI; CD89), which is the best characterized IgA receptor. FcαRI is expressed on monocytes, macrophages, granulocytes, subsets of dendritic cells, and Kupffer cells and binds both monomeric and dimeric IgA isoforms with median affinity. Binding of IgA to FcαRI mediates effector functions such as phagocytosis, oxidative burst, cytokine release, antigen presentation, and ADCC. In humans, two IgA isotypes, IgA1 and IgA2, and three allotypes, IgA2m(1), IgA2m(2) and IgA2n, have been distinguished. In some embodiments, IgA antibodies trigger polymorphonuclear cell (PMN) mediated ADCC more efficiently than IgG antibodies.

In some embodiments, an antibody construct described herein does not bind a B cell, a T cell, a platelet, and/or an erythrocyte. For example, in some embodiments, the IgA antibody can have low immunogenicity.

In some embodiments, an antibody construct described herein is a therapeutic antibody. In some embodiments, an antibody construct described herein can be a recombinant antibody. In some embodiments, an antibody construct described herein is made in a cell line. In some embodiments, the cell line is CHO. In some embodiments, the cell line is SP20. In some embodiments, the cell line is a HEK 239 cell line. In some embodiments, the HEK 293 cell line is HEK 293 F.

### IgA Antibody Modifications

Described herein are an antibody construct described herein comprising one or more amino acid substitution and/or one or more amino acid deletions in one or more IgA constant region domain.

In some embodiments, the amino acid numbering of an IgA antibody based construct described herein is indicated according to IMGT unique numbering for C-DOMAIN and C-LIKE-DOMAIN (as disclosed in “IMGT unique numbering for immunoglobulin and T cell receptor constant domains and Ig superfamily C-like domains.” Dev Comp Immunol. 2005; 29(3):185-203, the entire contents of which are incorporated by reference herein).

In some embodiments, the construct comprises a deletion of at least four glycosylation sites within the constant region. In some embodiments, the construct comprises a deletion of at least three N-linked glycosylation sites in the constant region of the antibody. In some embodiments, the antibody construct comprises a deletion of at least three N-linked glycosylation sites in the constant region of the antibody and at least one O-linked glycosylation site in the constant region of the antibody.

In some embodiments, is an antibody construct comprising an IgA constant region that comprise a deleted tail piece. In some embodiments, the construct comprises a deletion of the 3-20, 3-19, 3-18, 3-17, 3-16, 3-15, 3-14, 3-13, 3-12, 3-11, 3-10, 3-9, 3-8, 3-7, 3-6, 3-5, 3-4 C-terminal amino acids. In some embodiments, the construct comprises a deletion of the 3-20, 3-19, 3-18, 3-17, 3-16, 3-15, 3-14, 3-13, 3-12, 3-11, 3-10, 3-9, 3-8, 3-7, 3-6, 3-5, 3-4 C-terminal amino acids. In some embodiments, the C-terminal amino acids are from amino acids 131-148 of the IgA2 antibody, numbering according to IMGT scheme.

In some embodiments, the antibody construct comprises an IgA2 based constant region that comprises a deletion of amino acids 131-148, numbering according to IMGT scheme. In some embodiments, the antibody construct comprises an IgA2 based constant region that comprises a deletion of amino acids 147-148, numbering according to IMGT scheme. In some embodiments, the antibody construct comprises an IgA2 based constant region that comprises a deletion of amino acids 146-148, numbering according to IMGT scheme. In some embodiments, the antibody construct comprises an IgA2 based constant region that comprises a deletion of amino acids 145-148, numbering according to IMGT scheme. In some embodiments, the antibody construct comprises an IgA2 based constant region that comprises a deletion of amino acids 144-148, numbering according to IMGT scheme. In some embodiments, the antibody construct comprises an IgA2 based constant region that comprises a deletion of amino acids 143-148, numbering according to IMGT scheme. In some embodiments, the antibody construct comprises an IgA2 based constant region that comprises a deletion of amino acids 142-148, numbering according to IMGT scheme. In some embodiments, the antibody construct comprises an IgA2 based constant region that comprises a deletion of amino acids 141-148, numbering according to IMGT scheme. In some embodiments, the antibody construct comprises an IgA2 based constant region that comprises a deletion of amino acids 140-148, numbering according to IMGT scheme. In some embodiments, the antibody construct comprises an IgA2 based constant region that comprises a deletion of amino acids 139-148, numbering according to IMGT scheme. In some embodiments, the antibody construct comprises an IgA2 based constant region that comprises a deletion of amino acids 138-148, numbering according to IMGT scheme. In some embodiments, the antibody construct comprises an IgA2 based constant region that comprises a deletion of amino acids 137-148, numbering according to IMGT scheme. In some embodiments, the antibody construct comprises an IgA2 based constant region that comprises a deletion of amino acids 136-148, numbering according to IMGT scheme. In some embodiments, the antibody construct comprises an IgA2 based constant region that comprises a deletion of amino acids 135-148, numbering according to IMGT scheme. In some embodiments, the antibody construct comprises an IgA2 based constant region that comprises a deletion of amino acids 134-148, numbering according to IMGT scheme. In some embodiments, the antibody construct comprises an IgA2 based constant region that comprises a deletion of amino acids 133-148, numbering according to IMGT scheme. In some embodiments, the antibody construct comprises an IgA2 based constant region that comprises a deletion of amino acids 132-148, numbering according to IMGT scheme. In some embodiments, the antibody construct comprises an IgA2 based constant region that comprises a deletion of amino acids 131-148, numbering according to IMGT scheme. In some embodiments, the antibody construct comprises an IgA2 based constant region that comprises a deletion of amino acids P131-Y148, numbering according to IMGT scheme.

In some embodiments, the IgA antibody comprises a mutation of the C-terminal asparagine (N) amino acid. In some embodiments, the mutation is a non-conservative amino acid substitution. In some embodiments, the mutation deletes the glycosylation site of the C-terminal asparagine (N) amino acid of the IgA. In some embodiments, the antibody construct comprises an IgA2 based constant region that comprises a mutation of N135, numbering according to IMGT scheme. In some embodiments, the antibody construct comprises an IgA2 based constant region that comprises a non-conservative mutation of N135, numbering according to IMGT scheme. In some embodiments, the antibody construct comprises an IgA2 based constant region that comprises a N135Q mutation, numbering according to IMGT scheme.

In some embodiments, the antibody construct comprises an IgA2 based constant region that comprises a mutation of N45.2, numbering according to IMGT scheme. In some embodiments, the antibody construct comprises an IgA2 based constant region that comprises a non-conservative mutation of N45.2, numbering according to IMGT scheme. In some embodiments, the antibody construct comprises an IgA2 based constant region that comprises a N45.2G, numbering according to IMGT scheme. In some embodiments, the antibody construct comprising an IgA2 based constant region has an increased circulating half-life compared to an antibody construct comprising an IgA2 based constant region that does not have a mutation in the N45.2 amino acid.

In some embodiments, the antibody construct comprises an IgA2 based constant region that comprises a mutation of P124, numbering according to IMGT scheme. In some embodiments, the antibody construct comprises an IgA2 based constant region that comprises a non-conservative mutation of P124, numbering according to IMGT scheme. In some embodiments, the antibody construct comprises an IgA2 based constant region that comprises a P124R, numbering according to IMGT scheme. In some embodiments, the antibody construct comprising an IgA2 based constant region has an increased circulating half-life compared to an antibody construct comprising an IgA2 based constant region that does not have a mutation in the P124 amino acid. In some embodiments, the antibody construct comprising an IgA2 based constant region has an increased stability compared to an antibody construct comprising an IgA2 based constant region that does not have a mutation in the P124 amino acid.

In some embodiments, the antibody construct comprises an IgA2 based constant region that comprises a mutation of C92, numbering according to IMGT scheme. In some embodiments, the antibody construct comprises an IgA2 based constant region that comprises a non-conservative mutation of C92, numbering according to IMGT scheme. In some embodiments, the antibody construct comprises an IgA2 based constant region that comprises a C92S, numbering according to IMGT scheme. In some embodiments, the antibody construct comprising an IgA2 based constant region has a decreased aggregation compared to an IgA2 antibody that does not have a mutation in the C92 amino acid. In some embodiments, the antibody construct comprising an IgA2 based constant region has a decreased aggregation with serum proteins compared to an IgA2 antibody that does not have a mutation in the C92 amino acid. In some embodiments, the antibody construct has a decreased aggregation in vitro or in vivo compared to an IgA2 antibody that does not have a mutation in the C92 amino acid.

In some embodiments, antibody construct comprises an IgA2 based constant region that comprises a mutation of N120, numbering according to IMGT scheme. In some embodiments, antibody construct comprises an IgA2 based constant region that comprises a non-conservative mutation of N120, numbering according to IMGT scheme. In some embodiments, antibody construct comprises an IgA2 based constant region that comprises a N120T, numbering according to IMGT scheme. In some embodiments, the IgA2 antibody has an increased circulating half-life compared to an IgA2 antibody that does not have a mutation in the N120 amino acid.

In some embodiments, antibody construct comprises an IgA2 based constant region that comprises a mutation of 1121, numbering according to IMGT scheme. In some embodiments, antibody construct comprises an IgA2 based constant region that comprises a non-conservative mutation of 1121, numbering according to IMGT scheme. In some embodiments, antibody construct comprises an IgA2 based constant region that comprises an I121L, numbering according to IMGT scheme. In some embodiments, the IgA2 antibody has an increased circulating half-life compared to an IgA2 antibody that does not have a mutation in the N338 amino acid.

In some embodiments, antibody construct comprises an IgA2 based constant region that comprises a mutation of T122, numbering according to IMGT scheme. In some embodiments, antibody construct comprises an IgA2 based constant region that comprises a non-conservative mutation of T122, numbering according to IMGT scheme. In some embodiments, antibody construct comprises an IgA2 based constant region that comprises a T122S, numbering according to IMGT scheme. In some embodiments, the IgA2 antibody has an increased circulating half-life compared to an IgA2 antibody that does not have a mutation in the N339 amino acid.

In some embodiments, antibody construct comprises an IgA2 based constant region that comprises a mutation of C147, numbering according to IMGT scheme. In some embodiments, antibody construct comprises an IgA2 based constant region that comprises a non-conservative mutation of C147, numbering according to IMGT scheme. In some embodiments, antibody construct comprises an IgA2 based constant region that comprises a deletion of amino acid C147, numbering according to IMGT scheme. In some embodiments, the IgA2 antibody has a decreased aggregation compared to an IgA2 antibody that does not have a mutation in the C147 amino acid.

In some embodiments, antibody construct comprises an IgA2 based constant region that comprises a mutation of Y148, numbering according to IMGT scheme. In some embodiments, antibody construct comprises an IgA2 based constant region that comprises a non-conservative mutation of Y148, numbering according to IMGT scheme. In some embodiments, antibody construct comprises an IgA2 based constant region that comprises a deletion of amino acid Y148, numbering according to IMGT scheme.

In some embodiments, the IgA antibody comprises one or more albumin binding domains. In some embodiments, the one or more albumin binding domains are fused to a light chain or heavy chain of a IgA constant region. In some embodiments, the one or more albumin binding domains are fused to a heavy chain of a IgA constant region. In some embodiments, the one or more albumin binding domains are fused to a C-terminal region of a CH3 region of a heavy chain of a IgA constant region. In some embodiments, the IgA2 antibody has an increased circulating half-life compared to an IgA2 antibody that does not comprise one or more albumin binding domains. In some embodiments, antibody construct comprises an IgA2 based constant region that comprises one or more albumin binding domain and has circulating half-life within that of 1%, 5%, or 10% of a corresponding IgG antibody. In some embodiments, antibody construct comprises an IgA2 based constant region that comprises one or more albumin binding domain and has circulating half-life greater than that of a corresponding IgG antibody.

In some embodiments, the IgA antibody comprises one or more mutations described in Lohse S. et al. Cancer Res. 2015; 76(2):403-17; Meyer S. et al. mAbs. 2016; 8(1):87-98; or Leusen J. et al. Molecular Immunology. 2015; 68: 35-39.

In some embodiments, the one or more mutation or deletion as compared to a reference IgA sequence results in increased or decreased circulating half-life of the antibody construct. In some embodiments, the one or more mutation or deletion results in increased circulating half-life of the antibody construct. For example, the one or more mutations can increase the serum half-life of the antibody construct to up to 21 days or more in humans. Furthermore, the one or more mutations can increase the serum half-life of the antibody construct to up to 9 days or more in mice. In some embodiments, the one or more mutations can increase the serum half-life of the antibody construct to a level comparable to that of an immunoglobulin G (IgG) molecule. In some embodiments, the one or more mutation or deletion results in decreased circulating half-life of the antibody construct.

In some embodiments, the one or more mutations can increase the serum half-life of the antibody construct for at least about 7 days to about 30 days or more. In some embodiments, the one or more mutations can increase the serum half-life of the antibody construct for at least about 7 days. In some embodiments, the one or more mutations can increase the serum half-life of the antibody construct for at most about 30 days. In some embodiments, the one or more mutations can increase the serum half-life of the antibody construct for about 7 days to about 8 days, about 7 days to about 9 days, about 7 days to about 10 days, about 7 days to about 15 days, about 7 days to about 20 days, about 7 days to about 25 days, about 7 days to about 30 days, about 8 days to about 9 days, about 8 days to about 10 days, about 8 days to about 15 days, about 8 days to about 20 days, about 8 days to about 25 days, about 8 days to about 30 days, about 9 days to about 10 days, about 9 days to about 15 days, about 9 days to about 20 days, about 9 days to about 25 days, about 9 days to about 30 days, about 10 days to about 15 days, about 10 days to about 20 days, about 10 days to about 25 days, about 10 days to about 30 days, about 15 days to about 20 days, about 15 days to about 25 days, about 15 days to about 30 days, about 20 days to about 25 days, about 20 days to about 30 days, or about 25 days to about 30 days. In some embodiments, the one or more mutations can increase the serum half-life of the antibody construct for about 7 days, about 8 days, about 9 days, about 10 days, about 15 days, about 20 days, about 25 days, or about 30 days.

In some embodiments, the antibody construct exhibits increased stability. In some embodiments, the one or more mutation and/or one or more deletion results in increased stability of the antibody construct compared to a corresponding IgA antibody which does not comprise the one or mutation and/or one or more deletion.

In some embodiments, the antibody construct exhibits decreased aggregation. Antibody aggregation is a more common manifestation of physical instability. Protein aggregates generally have reduced activity and more importantly, greater immunogenicity potential because of the multiplicity of epitopes and/or conformational changes. Immunoglobulin aggregates are known to cause serious renal failure and anaphylactoid reactions such as headache, fever, and chills. It is therefore advantageous to decrease aggregation in antibody therapeutics. Additionally, the aggregate level in commercial intravenous immunoglobulin products is limited to less than 5% based on the World Health Organization (WHO) standards. In some embodiments, the one or more mutations results in decreased aggregation. In some embodiments, the one or more mutation and/or one or more deletion results in decreased aggregation of the antibody construct compared to a corresponding IgA antibody which does not comprise the one or mutation and/or one or more deletion.

In some embodiments, antibody constructs provided herein have an aggregate level ranging from at least about 0.1% to about 5% at most. In some embodiments, the antibody constructs provided herein have an aggregate level ranging from at least about 0.1%. In some embodiments, the antibody constructs provided herein have an aggregate level ranging from at most about 5%. In some embodiments, the antibody constructs provided herein have an aggregate level ranging from about 0.1% to about 0.5%, about 0.1% to about 1%, about 0.1% to about 2%, about 0.1% to about 3%, about 0.1% to about 4%, about 0.1% to about 5%, about 0.5% to about 1%, about 0.5% to about 2%, about 0.5% to about 3%, about 0.5% to about 4%, about 0.5% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the antibody constructs provided herein have an aggregate level ranging from about 0.1%, about 0.5%, about 1%, about 2%, about 3%, about 4%, or about 5%.

Therapeutic antibodies disclosed herein may comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

### CD47 and SIRPα

Signal-regulatory protein α (SIRP-α) is a protein widely expressed on the membrane of myeloid cells. SIRP-α interacts with CD47 (Cluster of Differentiation 47), a protein broadly expressed on many cell types in the body. The interaction of SIRP-α with CD47 prevents engulfment of “self” cells, which can otherwise be recognized by the immune system. It has been observed that high CD47 expression on tumor cells can act, in acute myeloid leukemia and several solid tumor cancers, as a negative prognostic factor for survival. Strategies focused on disrupting the interaction between CD47 and SIRP-α, such as the administration of agents that mask either CD47 or SIRP-α may be potential anticancer therapies.

Furthermore, checkpoint inhibition of the inhibitory receptor signal regulatory protein alpha (SIRPα) or its ligand CD47 has proven be very effective in pre-clinical models combined with IgG₁ anti-cancer therapies and certain of these CD47-SIRPa interaction blocking agents are already being tested in clinical trials for hematological and solid cancers (www.clinicaltrials.gov identifiers: NCT02216409; NCT02678338, NCT02641002; NCT02367196, NCT02890368; NCT02663518, NCT02953509). SIRPα is more or less selectively present on myeloid cells and limits ADCP/ADCC by macrophages and neutrophils, respectively. Its ubiquitously expressed ligand CD47 acts as a “don't eat me” signal and was found to be often overexpressed on cancer cells, inhibiting phagocytosis and clearance by macrophages. Indeed, in a clinical setting, the expression level of CD47 on cancer cells has been found to be inversely related to clinical response to anti-cancer antibody therapy. In several pre-clinical models, CD47-SIRPα blockade has proven to be a promising target for enhancing cancer immunotherapies when combined with IgG mAbs targeting different tumor antigens, including cetuximab and trastuzumab. However, for IgA antibodies directed against specific tumor antigens this enhancing effect of additional CD47-SIRPα checkpoint inhibition has not yet been investigated. Broad inhibition of CD47 can present a risk of adverse effects as CD47 is expressed ubiquitously in human cells. Thus, effective, localized targeting of CD47 on cancer cells is desired. In some embodiments, the therapeutic agents described herein have a low affinity binding to CD47; thus, preventing unwanted binding to cells other than cancer cells. In certain embodiments of constructs described herein, such a low affinity binding of CD47 is combined with a high affinity binding to an antigen expressed on an antigen binding cell to allow for the antigen binding to dictate the cellular specificity of the construct.

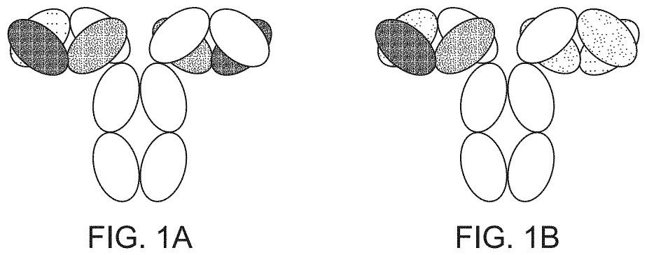

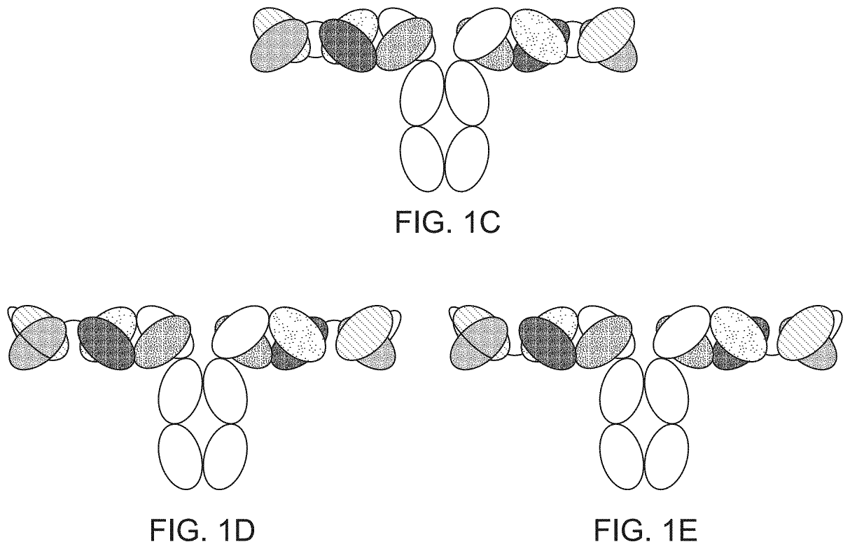

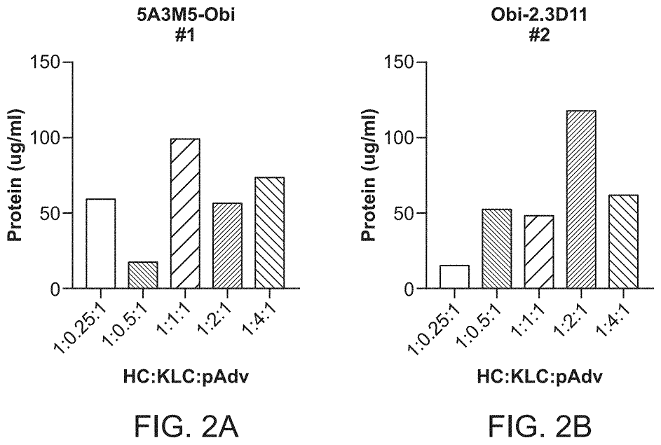

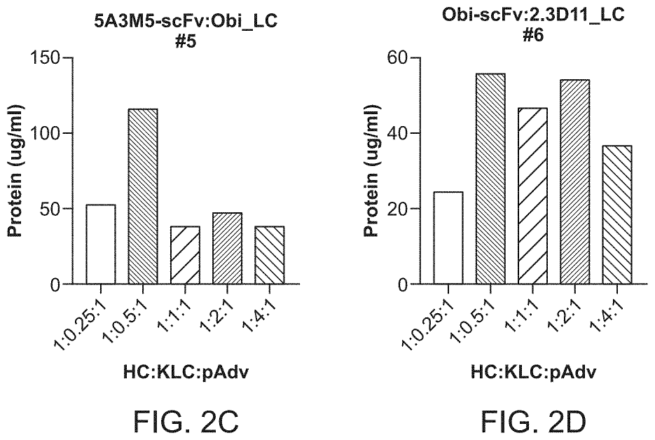

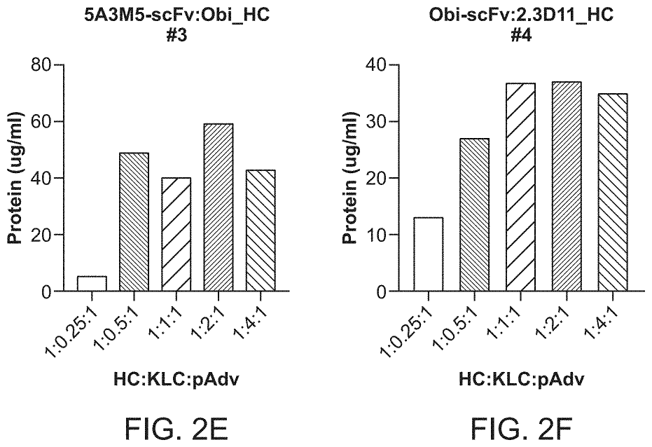

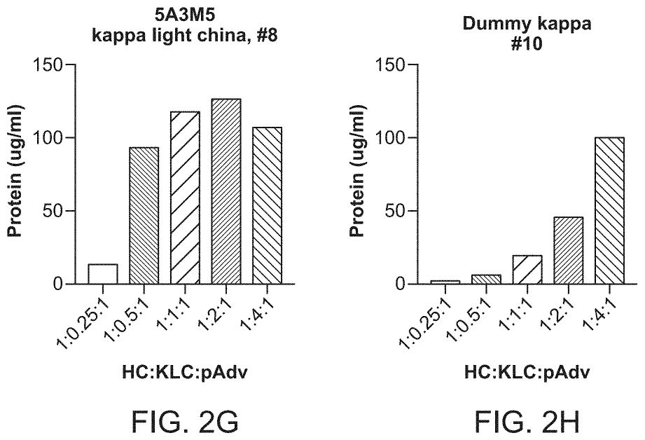

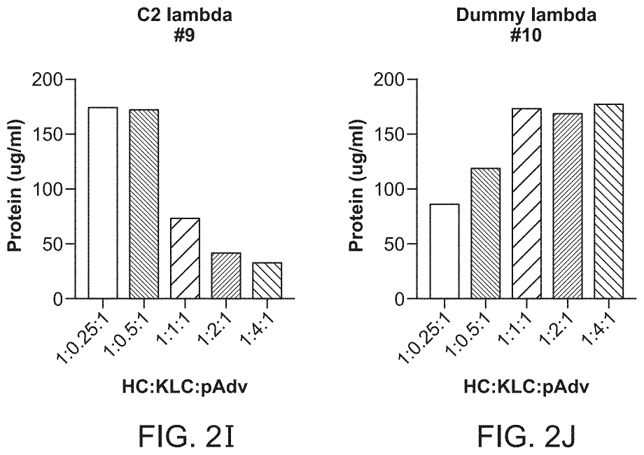

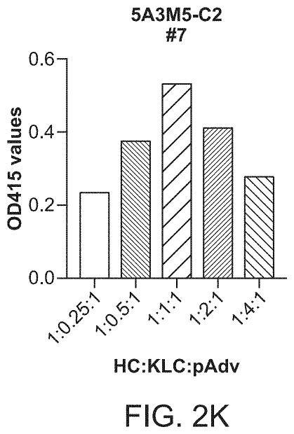

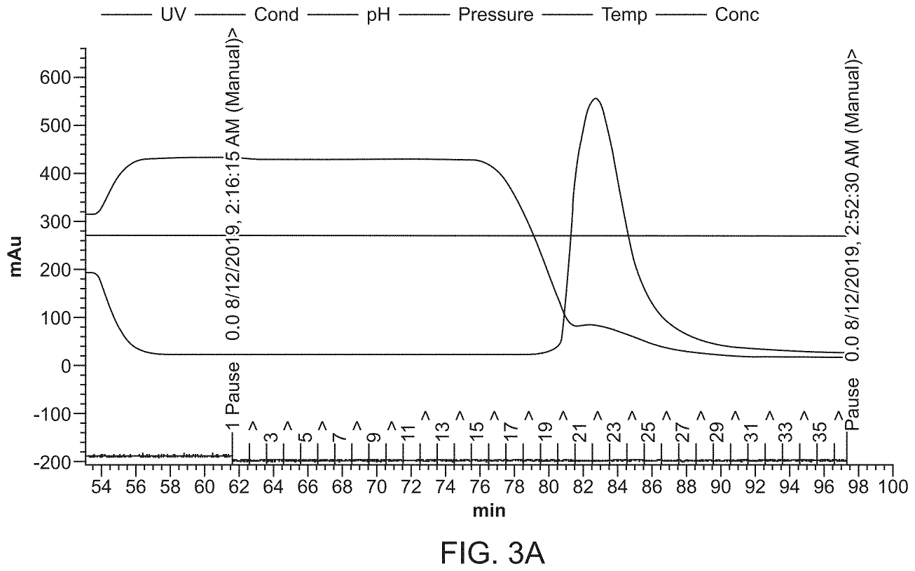

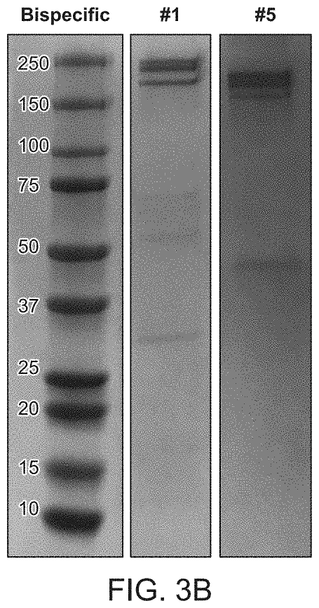

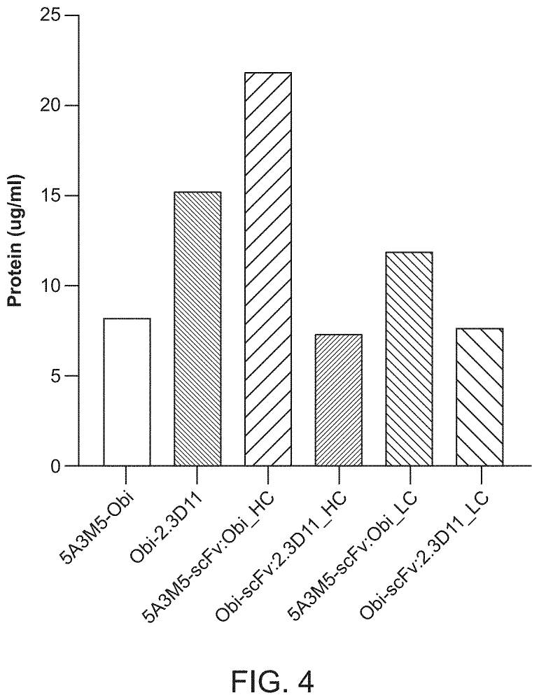

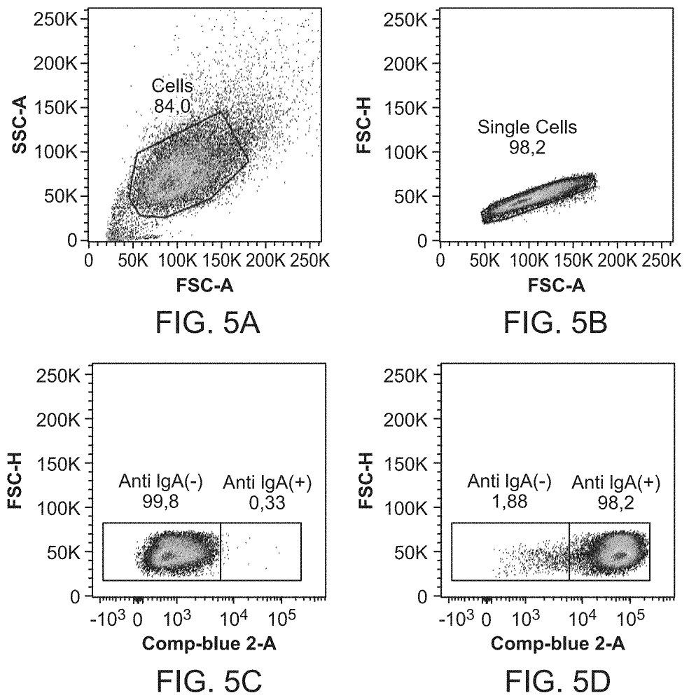

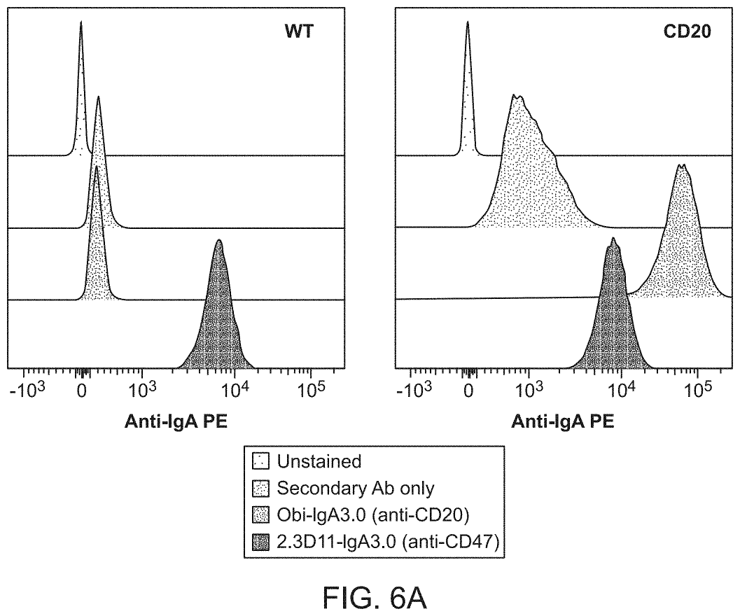

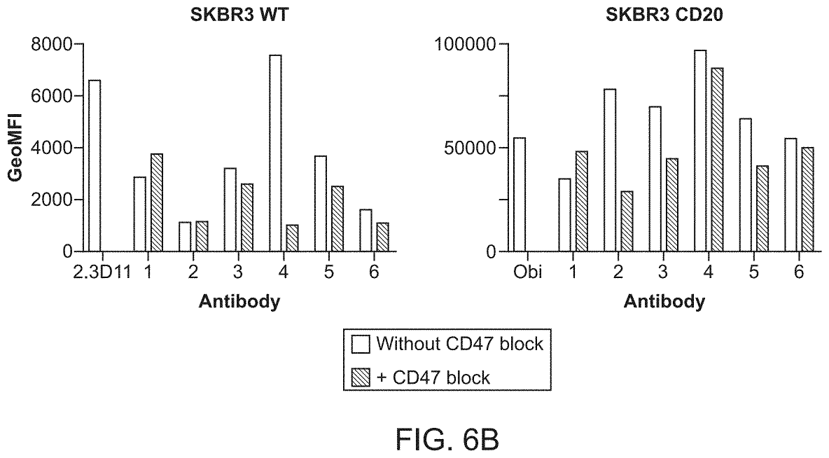

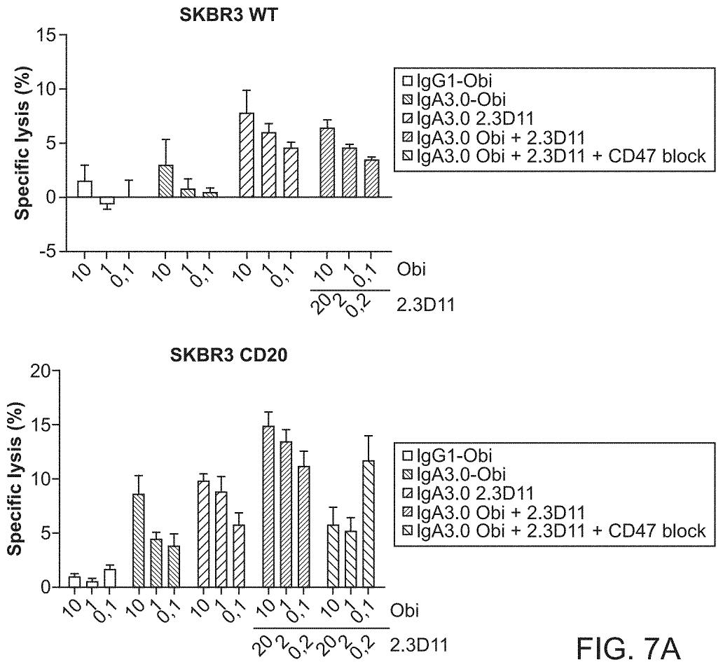

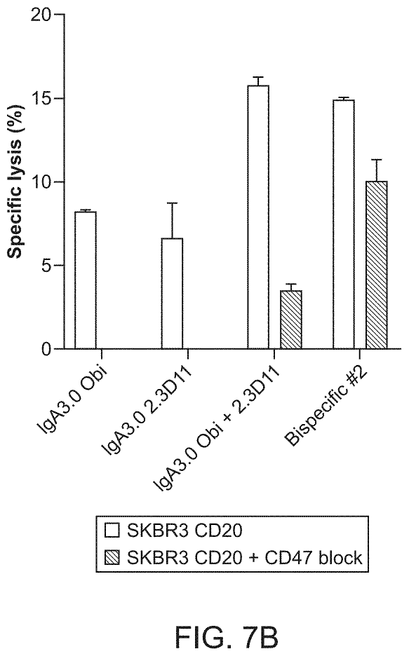

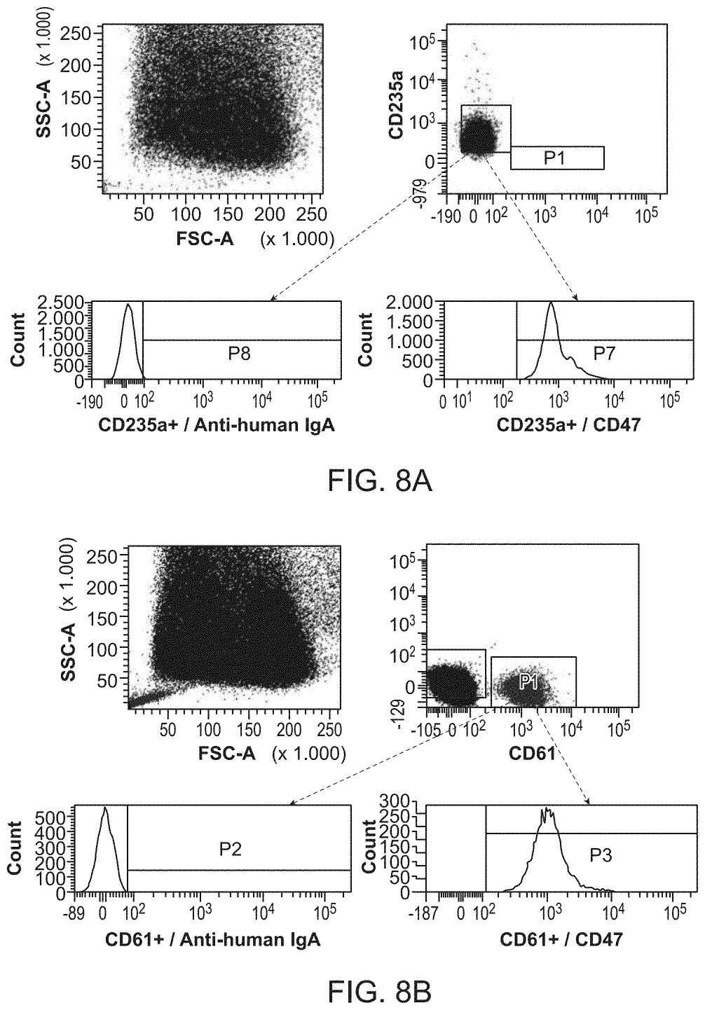

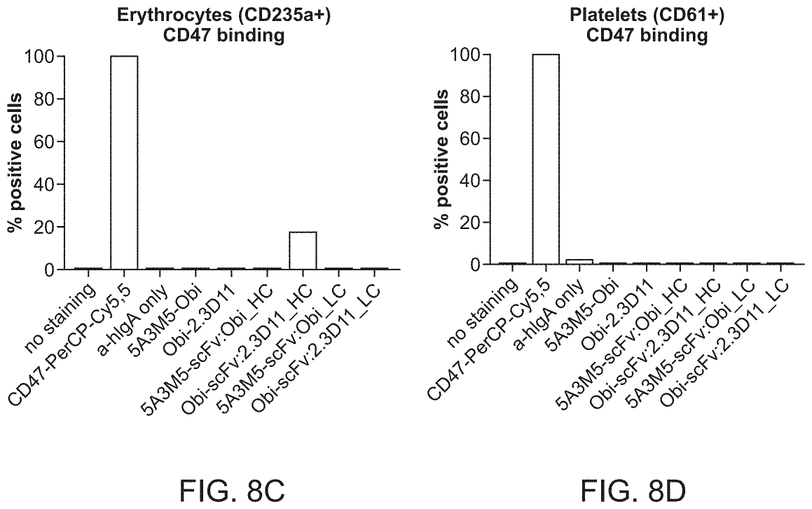

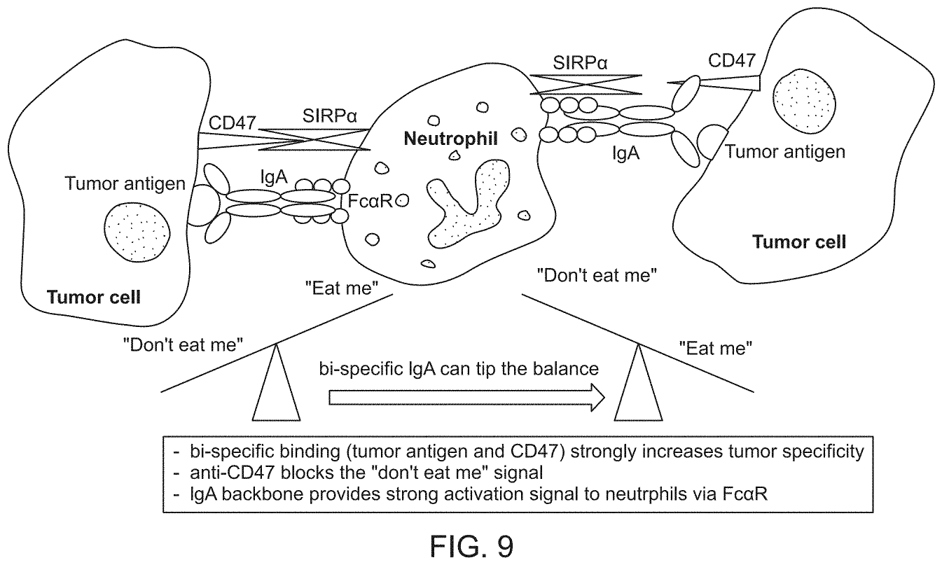

Provided herein are therapeutic agents comprising an IgA constant region domain, wherein the therapeutic agent binds CD47 and an additional tumor associated antigen, as shown in FIG. 1. Further provided herein are methods of treating a subject in need thereof, comprising administering to the subject a therapeutic dose of the therapeutic agents disclosed herein. Further provided herein, are pharmaceutical compositions comprising the therapeutic agents disclosed herein and a pharmaceutically acceptable carrier.

### Therapeutic Agents

Provided are therapeutic agents comprising antibody constructs described herein.

In embodiments described herein, are antibody constructs described herein that act as immune effector cell engager molecules. Such immune effector cell engagers comprise an immunoglobulin A (IgA) heavy chain domain based on IgA constructs as described herein, a CD47 binding domain as described herein; and an antigen binding domain which is expressed on antigen presenting cells; wherein the IgA heavy chain domain specifically binds a FcαR on an immune effector cell thereby engaging said immune effector cell, wherein the antigen binding domain binds an antigen on a target antigen presenting cell, wherein the CD47 binding domain inhibits binding of a CD47 expressed on a target antigen presenting cell with a signal regulatory protein α (SIRP α) on the immune effector cell thus allowing for the immune effector cell to destroy the antigen presenting cell, and wherein the antibody construct has a higher binding affinity for the antigen compared to the CD47, thereby selectively binding the antigen presenting cell.

In certain embodiments are method of inducing a neutrophil mediated immune response to a target antigen presenting cell comprising: contacting the target cell with an effective amount of an antibody construct, wherein the antibody construct comprises an immunoglobulin A (IgA) heavy chain domain; a CD47 binding domain; and an antigen binding domain;

wherein the antigen binding domain binds an antigen on the target cell, wherein the IgA heavy chain domain specifically binds a FcαR on a neutrophil thereby recruiting said neutrophil to the target cell, wherein the CD47 binding domain inhibits binding of a CD47 expressed on a target cell with a signal regulatory protein α (SIRP α) on the neutrophil, thereby facilitating the killing of the target cell by the neutrophil, and wherein the antibody construct has a higher binding affinity for the antigen compared to the CD47, thereby inducing the neutrophil mediated immune response, thereby selectively binding the antigen presenting target cell.

In some cases the target cell is a cancer cell that presents a tumor antigen. In cancer, there are four general groups of tumor antigens: (i) viral tumor antigens which can be identical for any viral tumor of this type, (ii) carcinogenic tumor antigens which can be specific for patients and for the tumors, (iii) isoantigens of the transplantation type or tumor-specific transplantation antigens which can be different in all individual types of tumor but can be the same in different tumors caused by the same virus; and (iv) embryonic antigens. As a result of the discovery of tumor antigens, tumor antigens have become important in the development of new cancer treatments that can specifically target the cancer. This has led to the development of antibodies directed against these tumor antigens. In addition to the development of antibodies against tumor antigens for cancer treatment, antibodies that target immune cells to boost the immune response have also been developed.

Disclosed herein, in certain embodiments, are therapeutic agents comprising an IgA constant region. In some cases, the therapeutic agent is an IgA antibody. The IgA constant region can be an IgA1 constant region. The IgA constant region can be an IgA2 constant region. For example, the therapeutic agent can be an IgA1 antibody or an IgA2 antibody. In some embodiments, the therapeutic agent does not bind a B cell, a T cell, a platelet, and/or an erythrocyte. For example, the therapeutic agent has low immunogenicity.

The therapeutic agent can be a bispecific or multispecific antibody. In some cases, the therapeutic agent co-engages two antigens at the cell surface. The therapeutic agent can co-engage two different antigens. In some examples, the binding of the therapeutic agent to two different antigens is sequential. For example, the binding of the therapeutic agent to the first antigen occurs first and thereby restricts the space explored by the second antibody arm. Consequentially, there can be a significant increase in local concentration of the second antigen, which can facilitate the binding of the second antibody arm.

In some embodiments, the therapeutic agent binds CD47 with low affinity as compared to the binding to an antigen. In some cases, the therapeutic agent reduces CD47 binding of a cancer cell. For example, the therapeutic agent can inhibit a human CD47 interaction with a signal regulatory protein α (SIRPα). Furthermore, the inhibition of interaction between the human CD47 and the SIRPα can increase a therapeutic potential of the therapeutic agent. The inhibition of interaction between the human CD47 and SIRPα can increase phagocytosis and clearance of cancer cells at a tumor site. For example, the cancer cells can be IgA-opsonized cancer cells. The therapeutic agent can be an IgA bispecific antibody comprising a low affinity CD47 arm. In some embodiments, the therapeutic agents described herein have a low affinity binding to CD47 that prevents the binding of the therapeutic agent to CD47 on a cell other than a cancer cell. In some cases, the low affinity CD47 arm of the therapeutic agent described herein binds to a tumor cell expressing CD47. In some examples, the low affinity CD47 arm of the therapeutic agent described herein does not bind to a cell expressing CD47 that is not a tumor cell. The therapeutic agent can be an IgA bispecific antibody that disrupts the CD47-SIRPα signal. In some examples, the therapeutic agent comprises an attenuated CD47 binding domain that binds to CD47 with a lower binding affinity (K_{d}) as compared to a corresponding wild type CD47 binding antibody.

In some embodiments, the therapeutic agent binds to CD47 with a binding affinity (K_{d}) of at least about 0.01 micromolar (04) to about 999 μM or more. In some embodiments, the therapeutic agent binds to CD47 with a binding affinity (K_{d}) of at least about 0.01 μM. In some embodiments, the therapeutic agent binds to CD47 with a binding affinity (K_{d}) of at most about 999 μM. In some embodiments, the therapeutic agent binds to CD47 with a binding affinity (K_{d}) of about 0.01 μM to about 0.1 μM, about 0.01 μM to about 0.5 μM, about 0.01 μM to about 1 μM, about 0.01 μM to about 5 μM, about 0.01 μM to about 10 μM, about 0.01 μM to about 50 μM, about 0.01 μM to about 100 μM, about 0.01 μM to about 200 μM, about 0.01 μM to about 300 μM, about 0.01 μM to about 500 μM, about 0.01 μM to about 999 μM, about 0.1 μM to about 0.5 μM, about 0.1 μM to about 1 μM, about 0.1 μM to about 5 μM, about 0.1 μM to about 10 μM, about 0.1 μM to about 50 μM, about 0.1 μM to about 100 μM, about 0.1 μM to about 200 μM, about 0.1 μM to about 300 μM, about 0.1 μM to about 500 μM, about 0.1 μM to about 999 μM, about 0.5 μM to about 1 μM, about 0.5 μM to about 5 μM, about 0.5 μM to about 10 μM, about 0.5 μM to about 50 μM, about 0.5 μM to about 100 μM, about 0.5 μM to about 200 μM, about 0.5 μM to about 300 μM, about 0.5 μM to about 500 μM, about 0.5 μM to about 999 μM, about 1 μM to about 5 μM, about 1 μM to about 10 μM, about 1 μM to about 50 μM, about 1 μM to about 100 μM, about 1 μM to about 200 μM, about 1 μM to about 300 μM, about 1 μM to about 500 μM, about 1 μM to about 999 μM, about 5 μM to about 10 μM, about 5 μM to about 50 μM, about 5 μM to about 100 μM, about 5 μM to about 200 μM, about 5 μM to about 300 μM, about 5 μM to about 500 μM, about 5 μM to about 999 μM, about 10 μM to about 50 μM, about 10 μM to about 100 μM, about 10 μM to about 200 μM, about 10 μM to about 300 μM, about 10 μM to about 500 μM, about 10 μM to about 999 μM, about 50 μM to about 100 μM, about 50 μM to about 200 μM, about 50 μM to about 300 μM, about 50 μM to about 500 μM, about 50 μM to about 999 μM, about 100 μM to about 200 μM, about 100 μM to about 300 μM, about 100 μM to about 500 μM, about 100 μM to about 999 μM, about 200 μM to about 300 μM, about 200 μM to about 500 μM, about 200 μM to about 999 μM, about 300 μM to about 500 μM, about 300 μM to about 999 μM, or about 500 μM to about 999 μM. In some embodiments, the therapeutic agent binds to CD47 with a binding affinity (K_{d}) of about 0.01 μM, about 0.1 μM, about 0.5 μM, about 1 μM, about 5 μM, about 10 μM, about 50 μM, about 100 μM, about 200 μM, about 300 μM, about 500 μM, or about 999 μM.

A construct described herein can comprise any CD47 binding domain from a known or de novo generated CD47 targeting antibody or molecule. These CD47 binding regions can be incorporated as is in a construct described herein or these can be further attenuated to reduce CD47 binding strength prior to incorporation in a construct described herein. Exemplary CDR regions of antibodies that can be used for CD47 targeting are provided in Table A below.

TABLE A
Exemplary human CD47 binding heavy and light
chain CDRs.

| | CDR- | | | | | CDR- |
|---|---|---|---|---|---|---|
| | H1 | CDR-H2 | CDR-H3 | CDR-L1 | CDR-L2 | L3 |
| CD47 | NYNMH | TIYPGNDD | GGYRAMDY | RSSQSIVY | KVSNRFS | FQGSH |
| | | TSYNQKFK | | SNGNTYLG | | VPYT |
| CD47 | GYGMS | TITSGGTY | SLAGNAMDY | RASQTISD | FASQSIS | QNGHG |
| | | TYYPDSVKG | | YLH | | FPRT |
| CD47 | GYTFT | YTDPRTDY | GGRVGLGY | RSSQNIVQ | KVFHRFS | FQGSH |
| | NYWIH | TEYNQKFK | | SNGNTYLE | | VPYT |
| CD47 | GYTFT | YIYPYNDGI | GGYYVPDY | RSRQSIVH | KVSNRFS | FQGSH |
| | NYVIH | LYNEKFKG | | TNGNTYLG | | VPYT |
| CD47 | GYSFT | YIDPLNGDT | GGKRAMDY | RASQDISN | YTSRLYS | QQGNT |
| | NYYIH | TYNQKFKG | | YLN | | LPWT |
| CD47 | GYTFT | IYPYNDGT | ARGGYYTY | QSLVHSNG | KVS | SQSTH |
| | NHV | | DD | KTY | | VPYT |

In some embodiments, the therapeutic agent binds to CD47 with a binding affinity (K_{d}) of about 1 mM to about 1,000 millimolar (mM). In some embodiments, the therapeutic agent binds to CD47 with a binding affinity (K_{d}) of at least about 1 mM. In some embodiments, the therapeutic agent binds to CD47 with a binding affinity (K_{d}) of at most about 1,000 mM. In some embodiments, the therapeutic agent binds to CD47 with a binding affinity (K_{d}) of about 1 mM to about 5 mM, about 1 mM to about 10 mM, about 1 mM to about 50 mM, about 1 mM to about 100 mM, about 1 mM to about 200 mM, about 1 mM to about 300 mM, about 1 mM to about 400 mM, about 1 mM to about 500 mM, about 1 mM to about 600 mM, about 1 mM to about 800 mM, about 1 mM to about 1,000 mM, about 5 mM to about 10 mM, about 5 mM to about 50 mM, about 5 mM to about 100 mM, about 5 mM to about 200 mM, about 5 mM to about 300 mM, about 5 mM to about 400 mM, about 5 mM to about 500 mM, about 5 mM to about 600 mM, about 5 mM to about 800 mM, about 5 mM to about 1,000 mM, about 10 mM to about 50 mM, about 10 mM to about 100 mM, about 10 mM to about 200 mM, about 10 mM to about 300 mM, about 10 mM to about 400 mM, about 10 mM to about 500 mM, about 10 mM to about 600 mM, about 10 mM to about 800 mM, about 10 mM to about 1,000 mM, about 50 mM to about 100 mM, about 50 mM to about 200 mM, about 50 mM to about 300 mM, about 50 mM to about 400 mM, about 50 mM to about 500 mM, about 50 mM to about 600 mM, about 50 mM to about 800 mM, about 50 mM to about 1,000 mM, about 100 mM to about 200 mM, about 100 mM to about 300 mM, about 100 mM to about 400 mM, about 100 mM to about 500 mM, about 100 mM to about 600 mM, about 100 mM to about 800 mM, about 100 mM to about 1,000 mM, about 200 mM to about 300 mM, about 200 mM to about 400 mM, about 200 mM to about 500 mM, about 200 mM to about 600 mM, about 200 mM to about 800 mM, about 200 mM to about 1,000 mM, about 300 mM to about 400 mM, about 300 mM to about 500 mM, about 300 mM to about 600 mM, about 300 mM to about 800 mM, about 300 mM to about 1,000 mM, about 400 mM to about 500 mM, about 400 mM to about 600 mM, about 400 mM to about 800 mM, about 400 mM to about 1,000 mM, about 500 mM to about 600 mM, about 500 mM to about 800 mM, about 500 mM to about 1,000 mM, about 600 mM to about 800 mM, about 600 mM to about 1,000 mM, or about 800 mM to about 1,000 mM. In some embodiments, the therapeutic agent binds to CD47 with a binding affinity (K_{d}) of about 1 mM, about 5 mM, about 10 mM, about 50 mM, about 100 mM, about 200 mM, about 300 mM, about 400 mM, about 500 mM, about 600 mM, about 800 mM, or about 1,000 mM.

In some cases, the therapeutic agent binds a tumor associated antigen. The therapeutic agent can be an IgA bispecific antibody comprising a high affinity tumor associated antigen arm. The tumor associated antigen can be CD20. The tumor associated antigen can be GD2. The tumor associated antigen can be mesothelin. The tumor associated antigen can be selected from the group consisting of GD2, CD38, CD19, EGFR, HER2, PD-L1, and CD25. The tumor associated antigen can be CD33, BCMA, CD44, α-Folate receptor, CAIX, CD30, ROR1, CEA, EGP-2, EGP-40, HER3, Folate-binding Protein, GD3, IL-13R-a2, KDR, EDB-F, mesothelin, CD22, EGFR, MUC-1, MAGE-A1, MUC16, h5T4, PSMA, TAG-72, EGFRvIII, CD123, VEGF-R2, or any combinations thereof.

A construct described herein can comprise any antigen binding domain from a known or de novo generated antigen targeting antibody or molecule. Exemplary CDR regions of antibodies that can be used for antigen targeting are provided in Table B below. The skilled artisan would know to utilize an appropriate antigen binding domain from known sources to appropriately target an antigen presenting cell.

TABLE B
Exemplary antigen binding heavy and light
chain CDRs.

| | CDR- | | | | CDR- | CDR- |
|---|---|---|---|---|---|---|
| | H1 | CDR-H2 | CDR-H3 | CDR-L1 | L2 | L3 |
| CD20 | GYAFS | IFPGDGDT | ARNVFD | KSLLHS | QMS | AQNLE |
| | YSW | | GYWLVY | NGITY | | LPYT |
| CD20 | GYTFT | IYPGNGDT | ARSTYYG | SSVSY | ATS | QQWTS |
| | SYN | | GDWYFNV | | | NPPT |
| CD20 | GFTFN | ISWNSGSI | AKDIQYGN | QSVSSY | DA | QRSNW |
| | DYA | | YYYGMDV | | | PIT |
| CD20 | GYAFS | IFPGDGDT | ARNVFDGY | KSLLHS | QMS | AQNLE |
| | YSW | | WLVY | NGITY | | LPYT |
| CD20 | GYTFT | IYPGNGDT | ARSTYYGG | SSVSY | ATS | QQWTS |
| | SYN | | DWYFNV | | | NPPT |
| CD20 | GFTFN | ISWNSGSI | AKDIQYGN | QSVSSY | DA | QRSNW |
| | DYA | | YYYGMDV | | | PIT |
| HER2 | GFNIK | IYPTNGYT | SRWGGDGF | QDVNTA | SAS | QQHYT |
| | DTY | | YAMDY | | | TPPT |
| HER2 | GFTFT | VNPNSGGS | ARNLGPSF | QDVSIG | SA | QQYYI |
| | DYT | | YFDYW | | | YPYT |
| HER2 | GFNIK | IYPTNGYT | SRWGGDGF | QDVNTA | SAS | QQHYT |
| | DTY | | YAMDY | | | TPPT |
| EGFR | GFSLT | IWSGGNT | ARALTYYD | QSIGTN | YAS | QQNNN |
| | NYG | | YEFAY | | | WPTT |
| EGFR | GGSVSS | IYYSGNT | VRDRVTGA | QDISNY | DAS | QHFDH |
| | GDYY | | FDI | | | LPLA |
| EGFR | GGSISS | IYYSGST | ARVSIFGV | QSVSSY | DAS | HQYGS |
| | GDYY | | GTFDY | | | TPLT |
| Meso- | GYSFT | ITPYNGAS | ARGGYDGR | SSVSY | DTS | QQWS |
| thelin | GYT | | GFDY | | | KHPLT |
| GD-2 | GSSFT | IDPYYGGT | VSGMEY | QSLVHR | KVS | SQSTH |
| | GYN | | | NGNTY | | VPPLT |

In some embodiments, the therapeutic agent described herein binds CD20. CD20 is a 33-37 kD, non-glycosylated phosphoprotein expressed on the surface of almost all normal and malignant B cells. CD20 knockout mice display an almost normal phenotype, suggesting a high level of redundancy. Recently, CD20 deficiency in humans was reported to result in impaired T cell-independent antibody responses. CD20 has been postulated to function as a calcium channel, and also to reside in lipid rafts, i.e. cholesterol-enriched microdomains in cell membranes. CD20 spans the membrane 4 times and possesses intracellular C- and N-termini. Two regions of CD20 are extracellular, forming a small and a large loop.

In some embodiments, the therapeutic agent described herein binds HER2. The HER2/neu (human epidermal growth factor receptor 2/receptor tyrosine-protein kinase erbB-2) is part of the human epidermal growth factor family. Overexpression of this protein can be shown to play an important role in the progression of cancer, for example, breast cancer. The HER2/neu protein can function as a receptor tyrosine kinase and autophosphorylates upon dimerization with binding partners. HER2/neu can activate several signaling pathways including, for example, mitogen-activated protein kinase, phosphoinositide 3-kinase, phospholipase C, protein kinase C, and signal transducer and activator of transcription (STAT). Examples of antibodies that can target and inhibit HER2/neu can include trastuzumab and pertuzumab.

In some embodiments, the therapeutic agent described herein binds EGFR. EGFR (epidermal growth factor receptor) encodes a member of the human epidermal growth factor family. Mutations that can lead to EGFR overexpression or over activity can be associated with a number of cancers, including squamous cell carcinoma and glioblastomas. EGFR can function as a receptor tyrosine kinase and ligand binding can trigger dimerization with binding partners and autophosphorylation. The phosphorylated EGFR can then activate several downstream signaling pathways including mitogen-activated protein kinase, phosphoinositide 3-kinase, phospholipase Cy, protein kinase C, and signal transducer and activator of transcription (STAT). Examples of antibodies that can target and inhibit EGFR can include cetuximab, panutumumab, nimotuzumab, and zalutumumab. One mutant variant of EGFR is EGFRvIII (epidermal growth factor receptor variant III). EGFRvIII can be the result of an EGFR gene rearrangement in which exons 2-7 of the extracellular domain are deleted. This mutation can result in a mutant receptor incapable of binding to any known ligand. The resulting receptor can engage in a constitutive low-level signaling and can be implicated in tumor progression. Examples of antibodies that can target EGFRvIII can include AMG595 and ABT806.

In some embodiments, the therapeutic agent described herein binds mesothelin. Mesothelin was originally described as the antigen recognized by the K1 monoclonal antibody that was generated after immunizing mice with the OVCAR-3 human ovarian carcinoma cell line. The mesothelin gene was then cloned in 1996. The mesothelin cDNA contains an open reading frame of 1884-bp and encodes a 69-kDa precursor protein (628 amino acids). After glycosylation, the precursor is cleaved by furin at amino acid 288-293 to yield a 40-kDa protein and a smaller 32-kDa fragment that is released from the cell. This 32-kDa shed fragment is called megakaryocyte-potentiating factor (MPF). The 40-kDa protein is found on the cell surface and can be released by treatment with phosphatidylinositol-specific phospholipase C. This 40-kDa GPI-linked membrane-bound protein was named mesothelin because it is produced by normal mesothelial cells. Since malignant mesotheliomas and ovarian adenocarcinomas are derived from normal mesothelial cells, it is not surprising that mesothelin is associated with these malignant diseases.

The most common form of mesothelin is membrane-bound, but 2 variants were found: Variant-1 with an 8 amino acid insertion is also membrane bound. Variant-2 is shed and soluble due to the lack of GPI-anchor signal sequences. Soluble mesothelin proteins are detectable in sera from patients with ovarian carcinoma and may provide a useful new marker for diagnosis of ovarian carcinoma and/or monitoring its response to therapy along with CA125 (cancer antigen-125). Moreover, soluble mesothelin is elevated in the blood and effusions of patients with mesothelioma and the determination of mesothelin levels in these fluids has been approved by the US FDA primarily as a tool for monitoring patient response and progression.

Mesothelin is suspected to play a role in cellular adhesion and tumor metastasis via its interaction with CA125, a tumor antigen used for diagnosis of ovarian cancer. CA125 is also named MUC16. CA125/MUC16 is a type I transmembrane protein expressed on the cell surfaces of many epithelia, and its soluble form can be released into extracellular space. In addition, mesothelin binding to CA125/MUC16 promotes pancreatic cancer cell motility and invasion via MMP-7 activation.

Mesothelin is considered a differentiation antigen because its expression in normal tissue is limited to mesothelia, but mesothelin is abundantly expressed in a variety of tumors including mesothelioma, ovarian cancer, pancreatic cancer and lung cancer. Mesothelin can be detected by immunohistological methods on normal mesothelial cells lining the pleural, pericardial, and peritoneal surfaces but not in any vital organs. It is often highly expressed in many epithelial cancers. Differential over-expression of mesothelin in tumors and its role in cell adhesion and tumor metastasis make mesothelin a suitable target for cancer therapy.

In some embodiments, the antigen binding moiety of a therapeutic agent described herein is specific to or binds GD2, CD38, CD19, EGFR, HER2, PD-L1, CD 25, CD33, BCMA, CD44, α-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, MUC-1, MUC-16, MAGE-A1, MUC16, h5T4, PSMA, TAG-72, EGFRvIII, CD123 and VEGF-R2. In some embodiments, the antigen binding domain comprises a single chain antibody fragment (scFv) comprising a variable domain light chain (VL) and variable domain heavy chain (VH) of a target antigen specific monoclonal antibody joined by a flexible linker, such as a glycine-serine linker or a Whitlow linker. In embodiments, the scFv is humanized. In some embodiments, the therapeutic agent comprises a variable domain an antibody. In some embodiments, the therapeutic agent comprises a variable domain from a human, humanized, or any other non-human origin IgG antibody. In some embodiments, the antigen binding moiety may comprise VH and VL that are directionally linked, for example, from N to C terminus, VH-linker-VL or VL-linker-VH. In some instances, the antigen binding domain recognizes an epitope of the target.

In one embodiment, the antigen binding moiety of a therapeutic agent described herein is specific to CD20. In one embodiment, the antigen binding moiety of a therapeutic agent described herein is specific to CD19. In one embodiment, the antigen binding moiety of a therapeutic agent described herein is specific to CD38. In one embodiment, the antigen binding moiety of a therapeutic agent described herein is specific to PD-L1. In one embodiment, the antigen binding moiety of a therapeutic agent described herein is specific to CD25. In one embodiment, the antigen binding moiety of a therapeutic agent described herein is specific to CD33. In another embodiment, the antigen binding moiety of a therapeutic agent described herein is specific to BCMA. In yet another embodiment, the antigen binding moiety of a therapeutic agent described herein is specific to CD44. In some embodiments, the antigen binding moiety of a therapeutic agent described herein is specific to α-Folate receptor. In some embodiments, the antigen binding moiety of a therapeutic agent described herein is specific to CAIX. In one embodiment, the antigen binding moiety of a therapeutic agent described herein is specific to CD30. In some embodiments, the antigen binding moiety of a therapeutic agent described herein is specific to ROR1. In one embodiment, the antigen binding moiety of a therapeutic agent described herein is specific to CEA. In some embodiments, the antigen binding moiety of a therapeutic agent described herein is specific to EGP-2. In one embodiment, the antigen binding moiety of a therapeutic agent described herein is specific to EGP-40. In another embodiment, the antigen binding moiety of a therapeutic agent described herein is specific to HER2. In yet another embodiment, the antigen binding moiety of a therapeutic agent described herein is specific to HER3. In yet another embodiment, the antigen binding moiety of a therapeutic agent described herein is specific to Folate-binding protein. In some embodiments, the antigen binding moiety of a therapeutic agent described herein is specific to GD2. In some embodiments, the antigen binding moiety of a therapeutic agent described herein is specific to GD3. In one embodiment, the antigen binding moiety of a therapeutic agent described herein is specific to IL-13R-a2. In one embodiment, the antigen binding moiety of a therapeutic agent described herein is specific to KDR. In one embodiment, the antigen binding moiety of a therapeutic agent described herein is specific to EDB-F. In another embodiment, the antigen binding moiety of a therapeutic agent described herein is specific to mesothelin. In yet another embodiment, the antigen binding moiety of a therapeutic agent described herein is specific to CD22. In one embodiment, the antigen binding moiety of a therapeutic agent described herein is specific to EGFR. In one embodiment, the antigen binding moiety of a therapeutic agent described herein is specific to MUC-1. In one embodiment, the antigen binding moiety of a therapeutic agent described herein is specific to MUC-16. In one embodiment, the antigen binding moiety of a therapeutic agent described herein is specific to MAGE-A1. In some embodiments, the antigen binding moiety of a therapeutic agent described herein is specific to h5T4. In some embodiments, the antigen binding moiety of a therapeutic agent described herein is specific to PSMA. In another embodiment, the antigen binding moiety of a therapeutic agent described herein is specific to TAG-72. In yet one embodiment, the antigen binding moiety of a therapeutic agent described herein is specific to EGFRvIII. In another embodiment, the antigen binding moiety of a therapeutic agent described herein is specific to CD123. In yet embodiment, the antigen binding moiety of a therapeutic agent described herein is specific to VEGF-R2.

Unaltered IgA, as a therapeutic monoclonal antibody, has a relatively short half-life. This is potentially due to a combination of the lack of binding to the neonatal Fc receptor (FcRn) in vivo and the low level of sialylation in the recombinant production system. The FcRn receptor has an established role in maintenance of antibody half-life, and decreased binding of an antibody molecule to FcRn translates into a shorter half-life in serum. In addition, IgA is more heavily glycosylated than IgG, therefore the enzyme (sialyl transferase) that transfers sialic acid to the nascent oligosaccharide is limited. In vivo, the Asialoglycoprotein Receptor (ASGPR) clears IgA more rapidly than IgG because of its low level of sialylation. Developing antibody therapeutics with extended half-life in blood serum is desirable for various reasons including, but not limited to improved therapeutic efficacy due to sustained circulation in the bloodstream and improved administration.

Provided herein are therapeutic agents comprising one or more mutations that result in improved pharmacokinetics (Lohse S. et al. Cancer Res. 2016; 76(2):403-17; Meyer S. et al. mAbs. 2016; 8(1):87-98). Provided herein are therapeutic agents comprising one or more albumin binding domains that result in improved pharmacokinetics. In some embodiments, the therapeutic agent comprises one or more mutations. The mutation can be a conservative mutation. The mutation can be a non-conservative mutation. In some embodiments, the therapeutic agent comprises one or more deletions. In some embodiments, the therapeutic agent comprises one or more albumin binding domains. The mutations or deletions can be found in the IgA constant region. Alternatively, the one or more mutations and/or the one or more deletions can be found in an IgA variable region. The one or more mutations can increase the serum half-life of the therapeutic agent to a level comparable to that of an immunoglobulin G (IgG) molecule. For example, the one or more mutations can increase the serum half-life of the therapeutic agent to up to 21 days or more in humans. Furthermore, the one or more mutations can increase the serum half-life of the therapeutic agent to up to 9 days or more in mice.

In some embodiments, the one or more mutations can increase the serum half-life of the therapeutic agent for at least about 7 days to about 30 days or more. In some embodiments, the one or more mutations can increase the serum half-life of the therapeutic agent for at least about 7 days. In some embodiments, the one or more mutations can increase the serum half-life of the therapeutic agent for at most about 30 days. In some embodiments, the one or more mutations can increase the serum half-life of the therapeutic agent for about 7 days to about 8 days, about 7 days to about 9 days, about 7 days to about 10 days, about 7 days to about 15 days, about 7 days to about 20 days, about 7 days to about 25 days, about 7 days to about 30 days, about 8 days to about 9 days, about 8 days to about 10 days, about 8 days to about 15 days, about 8 days to about 20 days, about 8 days to about 25 days, about 8 days to about 30 days, about 9 days to about 10 days, about 9 days to about 15 days, about 9 days to about 20 days, about 9 days to about 25 days, about 9 days to about 30 days, about 10 days to about 15 days, about 10 days to about 20 days, about 10 days to about 25 days, about 10 days to about 30 days, about 15 days to about 20 days, about 15 days to about 25 days, about 15 days to about 30 days, about 20 days to about 25 days, about 20 days to about 30 days, or about 25 days to about 30 days. In some embodiments, the one or more mutations can increase the serum half-life of the therapeutic agent for about 7 days, about 8 days, about 9 days, about 10 days, about 15 days, about 20 days, about 25 days, or about 30 days.

In some embodiments, the IgA constant region contains a mutation at position 166. The mutation can be a non-conservative mutation. For example, the mutation can comprise replacing asparagine at position 166 with glycine or a glycine homologue. In some cases, the amino acid replacement at position 166 increases a serum half-life of the therapeutic agent equal to or greater than the replacement with glycine. The therapeutic agent can have a greater half-life than a corresponding therapeutic agent comprising asparagine at position 166.

In some cases, the IgA constant region contains a mutation at position 337. The mutation can be a non-conservative mutation. For example, the mutation can comprise replacing asparagine at position 337 with threonine or a threonine homologue. In certain embodiments, the amino acid replacement at position 337 increases a serum half-life of the therapeutic agent equal to or greater than the replacement with asparagine. In some embodiments, the therapeutic agent has a greater half-life than a corresponding therapeutic agent comprising asparagine at position 337.

In some embodiments, the IgA constant region contains a mutation at position 338. The mutation can be a non-conservative mutation. For example, the mutation can comprise replacing isoleucine at position 338 with leucine or a leucine homologue. In certain cases, the amino acid replacement at position 338 increases a serum half-life of said therapeutic agent equal to or greater than the replacement with isoleucine. In some examples, the therapeutic agent has a greater half-life than a corresponding therapeutic agent comprising isoleucine at position 338.

In some embodiments, the IgA constant region contains a mutation at position 339. The mutation can be a non-conservative mutation. For example, the mutation can comprise replacing threonine at position 339 with serine or a serine homologue. In some cases, the therapeutic agent has a greater half-life than a corresponding therapeutic agent comprising threonine at position 339.

In certain embodiments, the IgA constant region lacks one or more glycosylation sites. In some cases, the IgA constant region comprises one or more albumin binding domains. In certain cases, one or more albumin binding domains are fused to a light chain or a heavy chain of said IgA constant region. In some embodiments, the one or more albumin binding domains increase a serum half-life of the therapeutic agent. In some cases, the therapeutic agent has a greater half-life than a corresponding therapeutic agent that does not comprise one or more albumin binding domains.

Developing antibody therapeutics with an increased stability is desirable as this can increase the shelf-life of the therapeutic while preserving the activity of the therapeutic intact. In some embodiments, the therapeutic agents provided herein have a stability that is the same or better than an IgG molecule. In some cases, the stability of the therapeutic agent is improved by introducing a mutation. In some embodiments, the IgA constant region contains a mutation at position 221. The mutation can be a non-conservative mutation. For example, the mutation can comprise replacing proline at position 221 with arginine or an arginine homologue. In some examples, the amino acid replacement has a greater stability of said therapeutic agent than a corresponding therapeutic agent comprising proline at position 221.

An additional inherent biophysical property of human antibodies is the propensity to aggregate. Antibody aggregation is a more common manifestation of physical instability. Protein aggregates generally have reduced activity and more importantly, greater immunogenicity potential because of the multiplicity of epitopes and/or conformational changes. Immunoglobulin aggregates are known to cause serious renal failure and anaphylactoid reactions such as headache, fever, and chills. It is therefore advantageous to decrease aggregation in antibody therapeutics. Additionally, the aggregate level in commercial intravenous immunoglobulin products is limited to less than 5% based on the World Health Organization (WHO) standards.

Provided herein are therapeutic agents comprising reduced aggregation. In some embodiments, the therapeutic agents provided herein have a decreased aggregation compared to that of an IgG molecule. In some embodiments, the therapeutic agents provided herein have a propensity to aggregate that is the same or better than an IgG molecule. In some cases, the propensity to aggregate of the therapeutic agent is improved by introducing a mutation. In some embodiments, IgA constant region contains a mutation at position 311. The mutation can be a non-conservative mutation. For example, the mutation can comprise replacing cysteine at position 311 with serine or a serine homologue. In some examples, the therapeutic agent has a decreased aggregation compared to a corresponding therapeutic agent comprising cysteine at position 311.

In some cases, the propensity to aggregate of the therapeutic agent is improved by introducing a deletion. In some cases, the IgA constant region contains a deletion at position 417. In some cases, the deletion of cysteine at position 471 can decrease a propensity to aggregate of the therapeutic agent. In certain examples, the therapeutic agent has a decreased aggregation compared to a corresponding therapeutic agent that does not comprise a deletion at position 471.

In some embodiments, the therapeutic agents provided herein have an aggregate level ranging from at least about 0.1% to about 5% at most. In some embodiments, the therapeutic agents provided herein have an aggregate level ranging from at least about 0.1%. In some embodiments, the therapeutic agents provided herein have an aggregate level ranging from at most about 5%. In some embodiments, the therapeutic agents provided herein have an aggregate level ranging from about 0.1% to about 0.5%, about 0.1% to about 1%, about 0.1% to about 2%, about 0.1% to about 3%, about 0.1% to about 4%, about 0.1% to about 5%, about 0.5% to about 1%, about 0.5% to about 2%, about 0.5% to about 3%, about 0.5% to about 4%, about 0.5% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the therapeutic agents provided herein have an aggregate level ranging from about 0.1%, about 0.5%, about 1%, about 2%, about 3%, about 4%, or about 5%.

Therapeutic agents disclosed herein may comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

A “multispecific antibody” is an antibody that can bind simultaneously to at least two targets that are of different structure, e.g., two different antigens, two different epitopes on the same antigen, or a hapten and/or an antigen or epitope. A “multivalent antibody” is an antibody that can bind simultaneously to at least two targets that are of the same or different structure. Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen. Specificity indicates how many antigens or epitopes an antibody is able to bind; i.e., monospecific, bispecific, trispecific, multispecific. Using these definitions, a natural antibody, e.g., an IgA, is bivalent because it has two binding arms but is monospecific because it binds to one epitope. Multispecific, multivalent antibodies are constructs that have more than one binding region of different specificity. For example, the bispecific antibody constructs disclosed herein have a CD47 binding region and an antigen binding region.

A “bispecific antibody” is an antibody that can bind simultaneously to two targets which are of different structure. Bispecific antibodies (bsAb) and bispecific antibody fragments (bsFab) may have at least one arm that specifically binds to, for example, a CD47, and at least one other arm that specifically binds to an antigen produced by or associated with a diseased cell, tissue, organ or pathogen, for example a tumor-associated antigen. A variety of bispecific antibodies can be produced using molecular engineering.

A bispecific antibody construct, or a composition described herein, is said to be administered in a “therapeutically effective amount” if the amount administered is physiologically significant. An agent is physiologically significant if its presence results in a detectable change in the physiology of a recipient subject. In particular embodiments, a bispecific antibody construct disclosed herein is physiologically significant if its presence invokes an antitumor response or mitigates the signs and symptoms of an infectious disease state. A physiologically significant effect could also be the evocation of a humoral and/or cellular immune response in the recipient subject leading to growth inhibition or death of target cells.

The term “linker” is used to denote polypeptides comprising two or more amino acid residues joined by peptide bonds and are used to link one or more antigen binding portions or variable domains. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). In some embodiments, the linker peptide comprises an amino acid sequence that is at least 90%, 91%, 92%, 93%, 94%, 95%, 96%, 97%, 98%, 99%, or 100% identical to the amino acid sequence of SEQ ID NO: 44. In some embodiments, the linker peptide comprises an amino acid sequence that is at least 90%, 91%, 92%, 93%, 94%, 95%, 96%, 97%, 98%, 99%, or 100% identical to the amino acid sequence of SEQ ID NO: 45.

An “Fv” or “Fv fragment” consists of only the light chain variable domain (VL) and heavy chain variable domain (VH) of a “single arm” of an immunoglobulin. Thus an “Fv” is the minimum antibody fragment which contains a complete antigen-recognition and binding site. A “two-chain” Fv fragment consists of a dimer of one heavy- and one light-chain variable domain in tight, non-covalent association. A single-chain Fv species (scFv) includes a VH and a VL domain of an immunoglobulin, with these domains being present in a single polypeptide chain in which they are covalently linked to each other by a linker peptide. Typically, in a scFv fragment the variable domains of the light and heavy chain associate in a dimeric structure analogous to that in a two-chain Fv species. In single chain Fv fragments, it is possible to either have the variable domain of the light chain arranged at the N-terminus of the single polypeptide chain, followed by the linker and the variable domain of the heavy chain arranged at the C-terminus of the polypeptide chain or vice versa, having the variable domain of the heavy chain arranged on the N-terminus and the variable domain of the light chain at the C-terminus with the linker peptide arranged in between. The linker peptide can be any flexible linker known in the art, for example, made from glycine and serine residues. It is also possible to additionally stabilize the domain association between the VH and the VL domain by introducing disulfide bonds into conserved framework regions (see Reiter et al. Stabilization of the Fv fragments in recombinant immunotoxins by disulfide bonds engineered into conserved framework regions, Biochemistry 1994, 33, 6551-5459). Such scFv fragments are also known as disulfide-stabilized scFv fragments (ds-scFv

### Binding to CD47

The bispecific antibody constructs that bind CD47 and fragments thereof serve to modulate, block, inhibit, reduce, antagonize, neutralize or otherwise interfere with the functional activity of CD47. Functional activities of CD47 include, by way of non-limiting example, interaction with SIRPα. The antibodies are considered to completely modulate, block, inhibit, reduce, antagonize, neutralize or otherwise interfere with the CD47-SIRPα interaction when the level of CD47-SIRPα interaction in the presence of the antibody is decreased by at least 50%, 60%, 70%, 80%, 90% or 95%, e.g., by 96%, 97%, 98%, 99% or 100% as compared to the level of CD47-SIRPα interaction in the absence of binding with a bispecific antibody described herein. The bispecific antibodies are considered to partially modulate, block, inhibit, reduce, antagonize, neutralize or otherwise interfere with the CD47-SIRPα interaction when the level of CD47-SIRPα interaction in the presence of the antibody is decreased by less than 95%, e.g., 10%, 20%, 25%, 30%, 40%, 50%, 60%, 75%, 80%, 85% or 90% as compared to the level of CD47-SIRPα interaction in the absence of binding with a bispecific antibody described herein.

The disclosure provides antibody constructs in which one binding region is specific for CD47, and other binding region is specific for an antigen (e.g., tumor antigen such as CD20). In some embodiments, the construct includes a functional IgA Fc portion, capable of binding a Fc receptor on a neutrophil. The antigen binding region of the bispecific antibody targets the CD47 binding region to the antigen presenting cell. The IgA Fc portion binds a neutrophil. The CD47 arm blocks, inhibits or otherwise reduces the interaction between CD47 and SIRPα, thereby inducing a neutrophil mediated immune response to the antigen. In some embodiments, the antigen binding region of the bispecific antibody includes an anti-CD20 antibody sequence or antigen-binding fragment thereof. Antigen binding region for use in a construct herein can target any antigen as described elsewhere in this disclosure. Such an antigen binding region can be obtained from known antibodies as well, for instance as shown in Table B.

In some embodiments the affinity of the antigen binding region is increased in the bispecific construct. In some embodiments, the affinity of the CD47 binding region is decreased in the bispecific construct. For example, in a bispecific antibody, the affinity of the antigen binding region is increased and the affinity of the CD47 binding region is decreased. These differences in the binding affinity of the antigen binding region and the CD47 binding region allows, for example, to improve selectivity for a target cell or group of target cells. CD47 binding region for use in a construct herein can be designed de novo, or obtained from known antibodies as well, for instance as shown in Table A.

In some embodiments, the affinity of the antigen binding region is increased by at least about: 2, 4, 5, 6, 7, 8, 9, 10, 20, 30, 40, 50, 60, 70, 80, 90, or 100 fold. In some embodiments, the increase is relative to a monospecific antigen binding antibody. In some embodiments, the increase is relative to the binding affinity of the CD47 binding region to CD47 in the bispecific antibody. In some embodiments, the affinity of the CD47 binding region is decreased by at least about: 2, 4, 5, 6, 7, 8, 9, 10, 20, 30, 40, 50, 60, 70, 80, 90, or 100 fold. In some embodiments, the decrease is relative to a monospecific CD47 binding antibody. In some embodiments, the decrease is relative to the binding affinity of the antigen binding region to antigen in the bispecific antibody.

### Methods of Making Antibodies

### General Antibody Techniques

Techniques for preparing monoclonal antibodies against virtually any target antigen are well known in the art. See, for example, Kohler and Milstein, Nature 256: 495 (1975), and Coligan et al. (eds.), CURRENT PROTOCOLS IN IMMUNOLOGY, VOL. 1, pages 2.5.1-2.6.7 (John Wiley & Sons 1991). Briefly, monoclonal antibodies can be obtained by injecting mice with a composition comprising an antigen, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

MAbs can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992).

After the initial raising of antibodies to the immunogen, the antibodies can be sequenced and subsequently prepared by recombinant techniques. Humanization and chimerization of murine antibodies and antibody fragments are well known to those skilled in the art. The use of antibody components derived from humanized, chimeric or human antibodies obviates potential problems associated with the immunogenicity of murine constant regions.

### Chimeric Antibodies

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. General techniques for cloning murine immunoglobulin variable domains are disclosed, for example, in Orlandi et al., Proc. Nat'l Acad. Sci. USA 86: 3833 (1989). Techniques for constructing chimeric antibodies are well known to those of skill in the art. As an example, Leung et al., Hybridoma 13:469 (1994), produced an LL2 chimera by combining DNA sequences encoding the Vκ and VH domains of murine LL2, an anti-CD22 monoclonal antibody, with respective human κ and IgG1 constant region domains.

### Humanized Antibodies

Techniques for producing humanized MAbs are well known in the art (see, e.g., Jones et al., Nature 321: 522 (1986), Riechmann et al., Nature 332: 323 (1988), Verhoeyen et al., Science 239: 1534 (1988), Carter et al., Proc. Nat'l Acad. Sci. USA 89: 4285 (1992), Sandhu, Crit. Rev. Biotech. 12: 437 (1992), and Singer et al., J. Immun. 150: 2844 (1993)). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al., Biotechnology 9:266 (1991) and Verhoeyen et al., Science 239: 1534 (1988). Generally, those human FR amino acid residues that differ from their murine counterparts and are located close to or touching one or more CDR amino acid residues would be candidates for substitution.

### Human Antibodies

Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Phamacol. 3:544-50). A fully human antibody also can be constructed by genetic or chromosomal transfection methods, as well as phage display technology, all of which are known in the art. See for example, McCafferty et al., Nature 348:552-553 (1990). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Recombinant Fab were cloned from the μ, γ and κ chain antibody repertoires and inserted into a phage display library (Id.). RNAs were converted to cDNAs and used to make Fab cDNA libraries using specific primers against the heavy and light chain immunoglobulin sequences (Marks et al., 1991, J Mol. Biol. 222:581-97). Library construction was performed according to Andris-Widhopf et al. (2000, In: PHAGE DISPLAY LABORATORY MANUAL, Barbas et al. (eds), 1st edition, Cold Spring Harbor Laboratory Press, Cold Spring Harbor, N.Y. pp. 9.1 to 9.22). The final Fab fragments were digested with restriction endonucleases and inserted into the bacteriophage genome to make the phage display library. Such libraries may be screened by standard phage display methods, as known in the art (see, e.g., Pasqualini and Ruoslahti, 1996, Nature 380:364-366; Pasqualini, 1999, The Quart. J. Nucl. Med. 43:159-162).

Phage display can be performed in a variety of formats, for their review, see e.g. Johnson and Chiswell, Current Opinion in Structural Biology 3:5564-571 (1993). Human antibodies may also be generated by in vitro activated B cells. See U.S. Pat. Nos. 5,567,610 and 5,229,275, incorporated herein by reference in their entirety. The skilled artisan will realize that these techniques are exemplary and any known method for making and screening human antibodies or antibody fragments may be utilized.

In another alternative, transgenic animals that have been genetically engineered to produce human antibodies may be used to generate antibodies against essentially any immunogenic target, using standard immunization protocols. Methods for obtaining human antibodies from transgenic mice are disclosed by Green et al., Nature Genet. 7:13 (1994), Lonberg et al., Nature 368:856 (1994), and Taylor et al., Int. Immun. 6:579 (1994). A non-limiting example of such a system is the XENOMOUSE® (e.g., Green et al., 1999, J. Immunol. Methods 231:11-23) from Abgenix (Fremont, Calif.). In the XENOMOUSE® and similar animals, the mouse antibody genes have been inactivated and replaced by functional human antibody genes, while the remainder of the mouse immune system remains intact.

The XENOMOUSE® was transformed with germline-configured YACs (yeast artificial chromosomes) that contained portions of the human IgH and Igkappa loci, including the majority of the variable region sequences, along accessory genes and regulatory sequences. The human variable region repertoire may be used to generate antibody producing B cells, which may be processed into hybridomas by known techniques. A XENOMOUSE® immunized with a target antigen will produce human antibodies by the normal immune response, which may be harvested and/or produced by standard techniques discussed above. A variety of strains of XENOMOUSE® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XENOMOUSE® system but may utilize any transgenic animal that has been genetically engineered to produce human antibodies.

### Antibody Cloning and Production

Various techniques, such as production of chimeric or humanized antibodies, may involve procedures of antibody cloning and construction. The antigen-binding Vκ (variable light chain) and VH (variable heavy chain) sequences for an antibody of interest may be obtained by a variety of molecular cloning procedures, such as RT-PCR, 5′-RACE, and cDNA library screening. The V genes of an antibody from a cell that expresses a murine antibody can be cloned by PCR amplification and sequenced. To confirm their authenticity, the cloned VL and VH genes can be expressed in cell culture as a chimeric Ab as described by Orlandi et al., (Proc. Natl. Acad. Sci. USA, 86: 3833 (1989)). Based on the V gene sequences, a humanized antibody can then be designed and constructed as described by Leung et al. (Mol. Immunol., 32: 1413 (1995)).

cDNA can be prepared from any known hybridoma line or transfected cell line producing a murine antibody by general molecular cloning techniques (Sambrook et al., Molecular Cloning, A laboratory manual, 2nd Ed (1989)). The Vκ sequence for the antibody may be amplified using the primers VK1BACK and VK1FOR (Orlandi et al., 1989) or the extended primer set described by Leung et al. (BioTechniques, 15: 286 (1993)). The VH sequences can be amplified using the primer pair VH1BACK/VH1FOR (Orlandi et al., 1989) or the primers annealing to the constant region of murine IgG described by Leung et al. (Hybridoma, 13:469 (1994)). Humanized V genes can be constructed by a combination of long oligonucleotide template syntheses and PCR amplification as described by Leung et al. (Mol. Immunol., 32: 1413 (1995)).

PCR products for Vκ can be subcloned into a staging vector, such as a pBR327-based staging vector, VKpBR, that contains an Ig promoter, a signal peptide sequence and convenient restriction sites. PCR products for VH can be subcloned into a similar staging vector, such as the pBluescript-based VHpBS. Expression cassettes containing the Vκ and VH sequences together with the promoter and signal peptide sequences can be excised from VKpBR and VHpBS and ligated into appropriate expression vectors, such as pKh and pG1g, respectively (Leung et al., Hybridoma, 13:469 (1994)). The expression vectors can be co-transfected into an appropriate cell and supernatant fluids monitored for production of a chimeric, humanized or human antibody. Alternatively, the Vκ and VH expression cassettes can be excised and subcloned into a single expression vector, such as pdHL2, as described by Gillies et al. Immunol. Methods 125:191 (1989) and also shown in Losman et al., Cancer, 80:2660 (1997)).

In an alternative embodiment, expression vectors may be transfected into host cells that have been pre-adapted for transfection, growth and expression in serum-free medium. Exemplary cell lines that may be used include the Sp/EEE, Sp/ESF and Sp/ESF-X cell lines (see, e.g., U.S. Pat. Nos. 7,531,327; 7,537,930 and 7,608,425; the Examples section of each of which is incorporated herein by reference). These exemplary cell lines are based on the Sp2/0 myeloma cell line, transfected with a mutant Bcl-EEE gene, exposed to methotrexate to amplify transfected gene sequences and pre-adapted to serum-free cell line for protein expression.

### Antibody Fragments

Antibody fragments which recognize specific epitopes can be generated by known techniques. Antibody fragments are antigen binding portions of an antibody, such as F(ab′)2, Fab′, F(ab)2, Fab, Fv, scFv and the like. F(ab′)2 fragments can be produced by pepsin digestion of the antibody molecule and Fab′ fragments can be generated by reducing disulfide bridges of the F(ab′)2 fragments. Alternatively, Fab′ expression libraries can be constructed (Huse et al., 1989, Science, 246:1274-1281) to allow rapid and easy identification of monoclonal Fab′ fragments with the desired specificity. F(ab)2 fragments may be generated by papain digestion of an antibody.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are described in U.S. Pat. Nos. 4,704,692; 4,946,778; Raag and Whitlow, FASEB 9:73-80 (1995) and Bird and Walker, TIBTECH, 9: 132-137 (1991).

Techniques for producing single domain antibodies (DABs or VHH) are also known in the art, as disclosed for example in Cossins et al. (2006, Prot Express Purif 51:253-259), incorporated herein by reference. Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; Maass et al., J Immunol Methods 324:13-25, 2007). The VHH may have potent antigen-binding capacity and can interact with novel epitopes that are inaccessible to conventional VH-VL pairs. (Muyldermans et al., 2001). Alpaca serum IgG contains about 50% camelid heavy chain only IgG antibodies (HCAbs) (Maass et al., 2007). Alpacas may be immunized with known antigens, such as TNF-α, and VHHs can be isolated that bind to and neutralize the target antigen (Maass et al., 2007). PCR primers that amplify virtually all alpaca VHH coding sequences have been identified and may be used to construct alpaca VHH phage display libraries, which can be used for antibody fragment isolation by standard biopanning techniques well known in the art (Maass et al., 2007). In certain embodiments, anti-pancreatic cancer VHH antibody fragments may be utilized in the claimed compositions and methods.

An antibody fragment can be prepared by proteolytic hydrolysis of the full length antibody or by expression in E. coli or another host of the DNA coding for the fragment. An antibody fragment can be obtained by pepsin or papain digestion of full length antibodies by conventional methods. These methods are described, for example, by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in METHODS IN ENZYMOLOGY VOL. 1, page 422 (Academic Press 1967), and Coligan at pages 2.8.1-2.8.10 and 2.10.-2.10.4.

### Bispecific Antibodies

Bispecific antibodies are antibodies that have binding specificities for at least two different antigens. In the present case, one of the binding specificities is for a target such as CD47 or any fragment thereof. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, and in Traunecker et al., EMBO J., 10:3655-3659 (1991).

Bispecific antibodies such as kappa lambda antibodies can be made using any of a variety of art-recognized techniques, including those disclosed in WO 2012/023053, the contents of which are hereby incorporated by reference in their entirety.

In other embodiments of producing bispecific antibodies, antibody variable domains with the desired binding specificities (antibody-antigen combining sites) can be linked to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

In some embodiments, the interface between a pair of antibody molecules in constructs herein can be engineered to maximize the percentage of heterodimers which are recovered from recombinant cell culture. The preferred interface includes at least a part of the CH3 region of an IgA antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991). Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Several strategies have been used to generate such bispecific molecules such as chemical cross-linking of antibody fragments, forced heterodimerization, quadroma technology, fusion of antibody fragments via polypeptide linkers and use of single domain antibodies. The availability of recombinant DNA technologies has lead to the generation of a multitude of bispecific antibody formats (see e.g., Ridgway J B et al. (1996) Protein Eng 9: 617-621). Linkers and mutations have frequently been introduced into different regions of the antibody to force heterodimer formation or to connect different binding moieties into a single molecule.

### Chemical Cross-Linking.

The use of chemical cross-linking reagents to covalently link two antibodies is a conceptually straightforward approach. Antibody fragments generated from their respective parent antibodies by enzymatic digestion or generated through recombinant technologies are conjugated using bifunctional reagents (Glennie M J et al., J Exp Med 1992; 175:217-225). Product homogeneity is the main limitation of this approach as the bispecific species has to be purified from homodimers and the modification steps can alter the integrity and stability of the proteins.

### Quadromas.

Quadromas and triomas can be generated by fusing either two hybridomas or one hybridoma with a B lymphocyte, respectively (Suresh M R et al., Methods Enzymol 1986; 121: 210-228). In this case the simultaneous expression of two heavy and two light chains leads to the random assembly of 10 antibody combinations and the desired bsAb represent only a small fraction of the secreted antibodies. The bsAb has to be purified using a combination of chromatographic techniques, and dramatically reduces production yields. A major limitation is that quadromas produce bsAb of rodent origin which limit their therapeutic potential due to immunogenicity issues.

### Recombinant Bispecific Antibodies

The majority of bispecific antibody formats can be generated by genetic engineering techniques using antibody fragment such as scFv or Fab fragments as building blocks connected via polypeptide linkers. Formats based on linked antibody fragments include tandem scFv (BiTE), diabodies and tandem-diabodies (Kipriyanov S M. Methods Mol Biol 2003; 207:323-333; Korn T et al, Int J Cancer 2002; 100:690-697). These building blocks can further be linked to an immunoglobulin Fc region given rise to ‘IgA-like’ molecules. These formats include diabody-Fc, tandem diabody-Fc, tandem diabody-CH3, (scFv)4-Fc and DVD-Ig (Lu D et al, J Immunol Methods 2003; 279: 219-232; Lu D et al, J Biol Chem 2005; 280: 19665-19672; Lu D et al, J Biol Chem 2004; 279: 2856-2865; Wu C et al., Nat Biotechnol 2007 25: 1290-7).

Strategies based on forcing the heterodimerization of two heavy chains have been explored. A first approach coined ‘knob into hole’ aims at forcing the pairing of two different IgG heavy chains by introducing mutations into the CH3 domains to modify the contact interface (Ridgway J B et al., Protein Eng 1996; 9: 617-621). On one chain amino acids with large side chains were introduced, to create a ‘knob’. Conversely, bulky amino acids were replaced by amino acids with short side chains to create a ‘hole’ into the other CH3 domain. By co-expressing these two heavy chains, more than 90% heterodimer formation was observed (‘knob-hole’) versus homodimers formation (‘hole-hole’ or ‘knob-knob’). A similar concept was developed using strand-exchange engineered domain (SEED) human CH3 domains based on human IgG and human IgA sequences (Davis J H et al., 2010, PEDS 23: 195-202). These engineered domains lead to the formation of heterodimeric molecules that can carry two different specificities.

Recently an improvement over the ‘knob into hole’ approach; “CrossMab.” has been described in WO 2009/080253 A1. This method involves the exchange of some of the light chain and heavy chain domains in addition to the ‘knob into hole’ mutations.

Single domain based antibodies. The immune systems of camelids (lamas and camels) and cartilaginous fish (nurse sharks) use single V-domains fused to a Fc demonstrating that a single domain can confer high affinity binding to an antigen. Camelid, shark and even human V domains represent alternatives to antibodies but they also be used for bsAbs generation. They can be reformatted into a classical IgG in which each arm has the potential to bind two targets either via its VH or VL domain.

The bispecific antibodies of the present disclosure can be made by any process disclosed in the application or otherwise known in the art.

### Dual Variable Domain Immunoglobulin

The bispecific antibody can comprise individually encoded peptides or “segments” which, in a single continuous chain, would comprise a compact tertiary structure. The component peptides are chosen so as to be asymmetric in their assumed structure, so as not to self-associate to form homo-multimers, but rather to associate in a complementary fashion, adopting a stable complex which resembles the parent tertiary structure. On the genetic level, these segments are encoded by interchangeable cassettes with suitable restriction sites. These standardized cassettes are fused C- or N-terminally to different recombinant proteins via a linker or hinge in a suitable expression vector system. Polypeptide segments which do not have the ability to assemble as homodimers are derived by cutting a parental polypeptide which has a compact tertiary structure. These polypeptide segments can then be fused to one or more different functional domains at the genetic level. These distinct polypeptide segments which are now fused to one or more functional domains can be, for example, coexpressed resulting in the formation of a native like parental structure attached to functional domains. This parental structure is formed by the dimerization of the polypeptide segments which were derived from the original parental polypeptide. The resulting multifunctional construct, would appear as a compact tertiary structure attached to the one or more functional domains. Once structural sub-domains are identified, the protein is dissected in such a way these sub-domains remain intact. As part of this disclosure, DNA sequences, vectors, preferably bicistronic vectors, vector cassettes, can be made and characterized in that they comprise a DNA sequence encoding an amino acid sequence and optionally at least one further (poly)peptide comprised in the multifunctional polypeptide of the invention, and additionally at least one, preferably singular cloning sites for inserting the DNA encoding at least one further functional domain or that they comprise DNA sequences encoding the amino acid sequences, and optionally the further (poly)peptide(s) comprised in the multifunctional polypeptide of the invention and suitable restriction sites for the cloning of DNA sequences encoding the functional domains, such that upon expression of the DNA sequences after the insertion of the DNA sequences encoding the functional domains into said restriction sites, in a suitable host the multifunctional polypeptide of the invention may be formed. Said vector cassette is characterized in that it comprises the inserted DNA sequence(s) encoding said functional domain(s) and host cells transformed with at least one vector or vector cassette of the invention which can be used for the preparation of said bispecific or multi-functional polypeptides. The host cell may be a mammalian, preferably human, yeast, insect, plant or bacterial, preferably E. coli cell. The bispecific antibodies can be prepared by a method which comprises culturing at least two host cells of the invention in a suitable medium, said host cells each producing only one of said first and said second amino acid sequences attached to at least one further functional domain, recovering the amino acid sequences, mixing thereof under mildly denaturing conditions and allowing in vitro folding of the multifunctional polypeptide of the invention from said amino acid sequences. The method may be characterized in that the further amino acid sequences attached to at least one further functional domain are/is produced by at least one further host cell not producing said first or second amino acid sequence. Additionally, the method may be characterized in that at least one further amino acid sequence attached to at least one further functional domain is produced by the host cell of the invention producing said first or second amino acid sequence.

When either the second or the first portion of an antibody construct described herein comprises two antibody variable domains, these two antibody variable domains can be a VH- and VL-domain which are associated with one another. However, it is also contemplated that the two antibody variable domains comprised in either the second or the first portion may be two VH domains or two VL regions which are associated with one another. In the event that the two antibody variable domains of the first or second portion are covalently associated with one another, the two antibody variable domains may be designed as an scFv fragment, meaning that the two domains are separated from one another by a peptide linker long enough to allow intermolecular association between these two domains. The design of linkers suitable for this purpose is described in the prior art, for example in the granted patents EP 623 679 B1, U.S. Pat. No. 5,258,498, EP 573 551 B1 and U.S. Pat. No. 5,525,491. In other words, a bispecific antibody may be a construct with a total of three antibody variable domains. One antibody variable domain specifically binds alone, i.e., without being paired with another antibody variable domain (a) either to a human immune effector cell by specifically binding to an effector antigen on the human immune effector cell or to a target cell, while the remaining two antibody variable domains together specifically bind (b) either to the target antigen on the target cell or to a human immune effector cell by specifically binding to an effector antigen on the human immune effector cell, respectively. In this case, the presence of three antibody variable domains in the bispecific antibody entails unique advantages. Often, an scFv exhibiting the desired binding specificity for a target antigen is already known and optimized, and omitting one of its two antibody variable domains would abolish or at least attenuate its binding characteristics. Such an scFv may make up part of an antibody construct described herein. Specifically, such a three-domain antibody may advantageously comprise an entire scFv as either its effector antigen- or target antigen-conferring portion. Effectively, then, this allows a bispecific antibody to be formed starting from a desired scFv by simple incorporation of only one additional antibody variable domain into the same polypeptide chain as the scFv, wherein the one additional antibody variable domain incorporated has an antigen binding specificity different than that of the scFv. The first and second portions of the bispecific antibody may be separated from one another by a synthetic polypeptide spacer moiety, which covalently (i.e., peptidically) links either the C-terminus of the first portion with the N-terminus of the second portion, or the C-terminus of the second portion with the N-terminus of the first portion. As such, the portions of these bispecific antibodies may be arranged, as either N-(first portion)-(second portion)-C or N-(second portion)-(first portion)-C. In some embodiments, binding sites of a second specificity are fused to the N- or C-terminus of the heavy or light chain, e.g., in the form of an scFv fragment or a variable single domain, resulting in bispecific, tetravalent molecules. Bispecific molecules generated through fusion of an scFv fragment to a mAb offer great flexibility. ScFv molecules can be linked to the N-terminus but also the C-terminus of the heavy or light chain variable domain of a mAb, generally without compromising productivity or antigen-binding activity. This group of bispecific molecules also includes DVD-Igs, where a second VH and VL domain is fused to the heavy and light chain, respectively, of a mAb, two-in-one antibodies, where a second specificity is introduced into the natural binding site of an IgG molecule, and mAb2 molecules, where a second specificity is built into the CH3 domain of the Fc region. A characteristic feature of all these molecules is a symmetry caused by dimeric assembly of two identical heavy chains, an intrinsic property of these chains.

Heavy chain heterodimerization can be achieved by engineering a charged CH3 interface to introduce an electrostatic steering effect or using the strand-exchange engineered domain technology (SEEDbody) with CH3 sequences composed of alternating segments from human IgA and IgG. In contrast to the bispecific IgG-like molecules, these bispecific antibodies are bivalent with a size basically identical to that of IgG. Fc heterodimerization was recently applied to generate a trivalent, bispecific molecule fusing a VH and a VL domain to the C-termini of the engineered heavy chains (HA-TF Fc variant.) Bispecific antibodies with a molecular mass in the range of 50-100 kDa can be generated by combining the variable domains of two antibodies. For example, two scFv have been connected by a more or less flexible peptide linker in a tandem orientation (tandem scFv, taFv, tascFv), which can be extended further by additional scFv, e.g., generating bispecific or trispecific triple bodies (sctb). Diabodies are heterodimeric molecules composed of the variable domains of two antibodies arranged either in the order VHA-VLB and VHB-VLA (VH-VL orientation) or in the order VLA-VHB and VLB-VHA (VL-VH orientation). The linker connecting the two domains within one chain is approximately 5 residues leading, after co-expression of the two chains within one cell, to a head-to-tail assembly and hence formation of a compact molecule with two functional binding sites. The diabody (Db) format was further stabilized by introducing interchain disulfide bonds (dsDb, DART molecules) or by generating a single-chain derivative (scDb). ScDbs can be converted into tetravalent molecules by reducing the middle linker, resulting in homodimerization of two chains. Small bispecific molecules have also been produced by fusing a scFv to the heavy or light chain of a Fab fragment. Furthermore, tandem scFv, diabodies and scDb have been fused to the Fc or a CH3 domain to generate tetravalent derivatives. Also, scFv can be combined with Fc or CH3 domains to generate tetravalent molecules, e.g., fusing scFvs to the N- and C-terminus of an Fc fragment, or using the knobs-into-holes approach to generate bivalent scFv-Fc or scFv-CH3 molecules. A different approach for the generation of bispecific antibodies of the present invention is the dock-and-lock method (DNL). Here, antibody fragments are fused to a homodimerizing docking domain (DDD) from human cAMP-dependent protein kinase A (PKA) and the anchoring domain (AD) from A-kinase anchor protein (AKAP) leading to the formation of bispecific, trivalent molecules. Many of the established bispecific antibody formats can also be combined with additional proteins and components, e.g., drugs, toxins, enzymes and cytokines, enabling dual targeting and delivery of a fusion partner. In addition, fusion to plasma proteins such as serum albumin or albumin-binding moieties can be applied to extend the plasma half-life of bispecific antibodies. Structure of Bispecific Antibodies

In one example the bispecific antibody may be a binding protein comprising a first polypeptide chain, wherein the polypeptide chain comprises VD-H1-(X1)n-VD-H2-C—(X2)n, wherein VD-H1 is a first heavy chain variable domain, VD-H2 is a second heavy chain variable domain, C is a constant domain, X1 represents a polypeptide linker, X2 represents an IgA Fc region and n is 0 or 1. In some embodiments The VD-H1 and VD-H2 in the binding protein may be heavy chain variable domains selected from the group consisting of a murine heavy chain variable domain, a human heavy chain variable domain, a CDR grafted heavy chain variable domain, and a humanized heavy chain variable domain. VD-H1 and VD-H2 may be capable of binding different antigens. C may be a heavy chain constant domain. For example, X1 is a linker peptide. For example, X1 is a linker listed herein. In an embodiment, X2 is an IgA Fc region. In another embodiment, X2 is a variant IgA Fc region. In some embodiments, VD-H1 is capable of binding CD47 and VD-H2 is capable of binding an antigen. In some embodiments, VD-H1 is capable of binding an antigen and VD-H2 is capable of binding a CD47.

In one example the bispecific antibody may be a binding protein comprising a second polypeptide chain, wherein the polypeptide chain comprises VD-L1-(X1)n-VD-L2-C—(X2)n, wherein VD-L1 is a first light chain variable domain, VD-L2 is a second light chain variable domain, C is a constant domain, X1 represents a polypeptide linker, X2 represents an IgA Fc region and n is 0 or 1. In some embodiments The VD-L1 and VD-L2 in the binding protein may be light chain variable domains selected from the group consisting of a murine light chain variable domain, a human light chain variable domain, a CDR grafted light chain variable domain, and a humanized light chain variable domain. VD-L1 and VD-L2 may be capable of binding different antigens. C may be a heavy chain constant domain. For example, X1 is a linker peptide. For example, X1 is a linker listed herein. In an embodiment, X2 is an IgA Fc region. In another embodiment, X2 is a variant IgA Fc region. In some embodiments, VD-L1 is capable of binding CD47 and VD-L2 is capable of binding an antigen. In some embodiments, VD-L1 is capable of binding an antigen and VD-L2 is capable of binding a CD47. In some embodiments, the bispecific antibody construct comprises both the first polypeptide chain and the second polypeptide chain. The bispecific antibodies of the present disclosure can be a dual-variable domain immunoglobulin (DVD-Ig™) as described in Jakob 2013 which combines the target binding domains of two monoclonal antibodies via flexible naturally occurring linkers, which yields a tetravalent IgG-like molecule.

The invention additionally provides a method of making a DVD-Ig binding protein by preselecting the parent antibodies of CD47 and a desired antigen (e.g., CD20). A method of making a Dual Variable Domain Immunoglobulin that binds two antigens comprises the steps of a) obtaining a first parent antibody, or antigen binding portion thereof, that binds a first antigen; b) obtaining a second parent antibody or antigen binding portion thereof, that binds a second antigen; c) constructing two copies of a first polypeptide chains, each of which comprises VD1-(X1)n-VD2-C—(X2)n, wherein, VD1 is a first heavy chain variable domain obtained from said first parent antibody, or antigen binding portion thereof; VD2 is a second heavy chain variable domain obtained from said second parent antibody or antigen binding portion thereof, which can be the same as or different from the first parent antibody; C is a heavy chain constant domain; (X1)n is a linker wherein said (X1)n is either present or absent; and (X2)n is an IgA Fc region, d) constructing two copies of a second polypeptide chains each of which comprises VD1-(X1)n-VD2-C—(X2)n, wherein, VD1 is a first light chain variable domain obtained from said first parent antibody, or antigen binding portion thereof; VD2 is a second light chain variable domain obtained from said second parent antibody, or antigen binding thereof, which can be the same as or different from the first parent antibody; C is a light chain constant domain; (X1)n is a linker, wherein said (X1)n is either present or absent; and (X2)n does not comprise an IgA Fc region, wherein said (X2)n is either present or absent; and e) expressing two copies of said first and second polypeptide chains; such that a DVD-Ig binds said first antigen (e.g., CD47) and said second antigen (e.g., CD20) is generated.

Generation of Antigen Binding and/or CD47 Binding Domains:

The variable domains of the DVD binding protein can be obtained from parent antibodies, including polyclonal and mAbs that bind antigens of interest. These antibodies may be naturally occurring or may be generated by recombinant technology, or can be designed de novo. MAbs can be prepared using a wide variety of techniques known in the art including the use of hybridoma, recombinant, and phage display technologies, or a combination thereof. Monoclonal antibodies can be prepared by methods disclosed herein.

The dual variable domain immunoglobulin (DVD-Ig) molecule is designed such that two different light chain variable domains (VL) from the two different parent monoclonal antibodies are linked in tandem directly or via a short linker by recombinant DNA techniques, followed by the light chain constant domain, and optionally, an Fc region. Similarly, the heavy chain comprises two different heavy chain variable domains (VH) linked in tandem, followed by the constant domain CH1 and Fc region. The variable domains can be obtained using recombinant DNA techniques from a parent antibody generated by any one of the methods described herein. The variable domain may be a murine heavy or light chain variable domain, a CDR a human heavy or light chain variable domain. The first and second variable domains may be linked directly to each other using recombinant DNA techniques, linked via a linker sequence, or the two variable domains are linked. The variable domains may bind the same antigen or may bind different antigens. The constant domain may be linked to the two linked variable domains using recombinant DNA techniques. Sequence comprising linked heavy chain variable domains may be linked to a heavy chain constant domain and sequence comprising linked light chain variable domains is linked to a light chain constant domain. The constant domains may also be human heavy chain constant domain and human light chain constant domain respectively. The DVD heavy chain may be further linked to an IgA Fc region. The Fc region may be a native sequence Fc region, or a variant Fc region, or a human Fc region, or a Fc region from IgA1, IgA2. Two heavy chain DVD polypeptides and two light chain DVD polypeptides may be combined to form a DVD-Ig molecule.

The design of the “dual-specific multivalent full length binding proteins” of the present invention leads to a dual variable domain light chain and a dual variable domain heavy chain which assemble primarily to the desired “dual-specific multivalent full length binding proteins”.

### Construction of DVD Molecules

The dual variable domain immunoglobulin (DVD-Ig) molecule is designed such that two different light chain variable domains (VL) from the two parent monoclonal antibodies, which can be the same or different, are linked in tandem directly or via a short linker by recombinant DNA techniques, followed by the light chain constant domain, and optionally, an IgA Fc region. Similarly, the heavy chain comprises two different heavy chain variable domains (VH) linked in tandem, followed by the constant domain CH1 and IgA Fc region

The variable domains can be obtained using recombinant DNA techniques from a parent antibody generated by any one of the methods described herein. In an embodiment, the variable domain is a murine heavy or light chain variable domain. In another embodiment, the variable domain is a CDR grafted or a humanized variable heavy or light chain domain. In an embodiment, the variable domain is a human heavy or light chain variable domain.

In one embodiment the first and second variable domains are linked directly to each other using recombinant DNA techniques. In another embodiment the variable domains are linked via a linker sequence. In an embodiment, two variable domains are linked. Three or more variable domains may also be linked directly or via a linker sequence. The variable domains may bind the same antigen or may bind different antigens. DVD-Ig molecules of the invention may include one immunoglobulin variable domain and one non-immunoglobulin variable domain, such as ligand binding domain of a receptor, or an active domain of an enzyme. DVD-Ig molecules may also comprise two or more non-Ig domains.

In an embodiment a constant domain is linked to the two linked variable domains using recombinant DNA techniques. In an embodiment, sequence comprising linked heavy chain variable domains is linked to a heavy chain constant domain and sequence comprising linked light chain variable domains is linked to a light chain constant domain. In an embodiment, the constant domains are human heavy chain constant domain and human light chain constant domain respectively. In an embodiment, the DVD heavy chain is further linked to an Fc region. The Fc region may be a native sequence Fc region, or a variant Fc region. In another embodiment, the Fc region is a human Fc region. In another embodiment the Fc region includes Fc region from IgG1, IgG2, IgG3, IgG4, IgA, IgM, IgE, or IgD.

In another embodiment two heavy chain DVD polypeptides and two light chain DVD polypeptides are combined to form a DVD-Ig molecule.

Binding proteins of the present invention may be produced by any of a number of techniques known in the art. For example, expression from host cells, wherein expression vector(s) encoding the DVD heavy and DVD light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the DVD proteins of the invention in either prokaryotic or eukaryotic host cells, DVD proteins are expressed in eukaryotic cells, for example, mammalian host cells, because such eukaryotic cells (and in particular mammalian cells) are more likely than prokaryotic cells to assemble and secrete a properly folded and immunologically active DVD protein.

Exemplary mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in Kaufman, R. J. and Sharp, P. A. (1982) Mol. Biol. 159:601-621), NS0 myeloma cells, COS cells, SP2 and PER.C6 cells. When recombinant expression vectors encoding DVD proteins are introduced into mammalian host cells, the DVD proteins are produced by culturing the host cells for a period of time sufficient to allow for expression of the DVD proteins in the host cells or secretion of the DVD proteins into the culture medium in which the host cells are grown. DVD proteins can be recovered from the culture medium using standard protein purification methods.

In an exemplary system for recombinant expression of DVD proteins in constructs described herein, a recombinant expression vector encoding both the DVD heavy chain and the DVD light chain is introduced into dhfr-CHO cells by calcium phosphate-mediated transfection. Within the recombinant expression vector, the DVD heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the DVD heavy and light chains and intact DVD protein is recovered from the culture medium. Standard molecular biology techniques are used to prepare the recombinant expression vector, transfect the host cells, select for transformants, culture the host cells and recover the DVD protein from the culture medium. Still further the invention provides a method of synthesizing a DVD protein of the invention by culturing a host cell of the invention in a suitable culture medium until a DVD protein of the invention is synthesized. The method can further comprise isolating the DVD protein from the culture medium.

An important feature of DVD-Ig is that it can be produced and purified in a similar way as a conventional antibody. The production of DVD-Ig results in a homogeneous, single major product with desired dual-specific activity, without any sequence modification of the constant region or chemical modifications of any kind. Other previously described methods to generate “bi-specific”, “multi-specific”, and “multi-specific multivalent” full length binding proteins do not lead to a single primary product but instead lead to the intracellular or secreted production of a mixture of assembled inactive, mono-specific, multi-specific, multivalent, full length binding proteins, and multivalent full length binding proteins with combination of different binding sites. As an example, based on the design described by Miller and Presta (PCT Publication No. WO2001/077342(A1), there are 16 possible combinations of heavy and light chains. Consequently only 6.25% of protein is likely to be in the desired active form, and not as a single major product or single primary product compared to the other 15 possible combinations. Separation of the desired, fully active forms of the protein from inactive and partially active forms of the protein using standard chromatography techniques, typically used in large scale manufacturing, is yet to be demonstrated.

The design of the “dual-specific multivalent full length binding proteins” for use in constructs described herein leads to a dual variable domain light chain and a dual variable domain heavy chain which assemble primarily to the desired “dual-specific multivalent full length binding proteins”.

At least 50%, at least 75% and at least 90% of the assembled, and expressed dual variable domain immunoglobulin molecules are the desired dual-specific tetravalent protein. This aspect of the invention particularly enhances the commercial utility of the invention. Therefore, the present invention includes a method to express a dual variable domain light chain and a dual variable domain heavy chain in a single cell leading to a single primary product of a “dual-specific tetravalent full length binding protein”.

Provided herein are methods of expressing a dual variable domain light chain and a dual variable domain heavy chain in a single cell leading to a “primary product” of a “dual-specific tetravalent full length binding protein,” where the “primary product” is more than 50% of all assembled protein, comprising a dual variable domain light chain and a dual variable domain heavy chain.

Provided herein are methods of expressing a dual variable domain light chain and a dual variable domain heavy chain in a single cell leading to a single “primary product” of a “dual-specific tetravalent full length binding protein,” where the “primary product” is more than 75% of all assembled protein, comprising a dual variable domain light chain and a dual variable domain heavy chain.

Provided herein are methods of expressing a dual variable domain light chain and a dual variable domain heavy chain in a single cell leading to a single “primary product” of a “dual-specific tetravalent full length binding protein,” where the “primary product” is more than 90% of all assembled protein, comprising a dual variable domain light chain and a dual variable domain heavy chain.

### Kappa-Lambda Bodies

In some embodiments, provided herein are bispecific antibodies in the kappa-lambda antibody format. The bispecific antibodies provided herein have a common heavy chain, two light chains—one Kappa (K), one Lambda (λ)—that each has a different specificity (i.e., two light chains, two specificities). The methods provided herein produce molecules having specific binding where diversity is restricted to the VL region. These methods produce the bispecific antibodies through controlled co-expression of the three chains (one VH chains, two VL chains), and purification of the bispecific antibody

This type of molecule is composed of two copies of a unique heavy chain polypeptide, a first light chain variable region fused to a constant Kappa domain and second light chain variable region fused to a constant Lambda domain. Each combining site displays a different antigen specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However it is also possible to obtain bispecific antibodies of the invention by fusing a Kappa light chain variable domain to a constant Lambda domain for a first specificity and fusing a Lambda light chain variable domain to a constant Kappa domain for the second specificity.

An essential step of the method is the identification of two antibody Fv regions (each composed by a variable light chain and variable heavy chain domain) having different antigen specificities that share the same heavy chain variable domain. Numerous methods have been described for the generation of monoclonal antibodies and fragments thereof (See, e.g., Antibodies: A Laboratory Manual, Harlow E, and Lane D, 1988, Cold Spring Harbor Laboratory Press, Cold Spring Harbor, N.Y., incorporated herein by reference). Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique (see Kozbor, et al., 1983 Immunol Today 4: 72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al, 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

Monoclonal antibodies are generated, e.g., by immunizing an animal with a target antigen or an immunogenic fragment, derivative or variant thereof. Alternatively, the animal is immunized with cells transfected with a vector containing a nucleic acid molecule encoding the target antigen, such that the target antigen is expressed and associated with the surface of the transfected cells. A variety of techniques are well-known in the art for producing xenogeneic non-human animals. For example, see U.S. Pat. Nos. 6,075,181 and 6,150,584, which is hereby incorporated by reference in its entirety.

Alternatively, the antibodies are obtained by screening a library that contains antibody or antigen binding domain sequences for binding to the target antigen. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”).

Hybridomas resulting from myeloma/B cell fusions are then screened for reactivity to the target antigen. Monoclonal antibodies are prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Kappa-lambda antibodies having the same heavy chain variable domain can be generated by the use of antibody libraries in which the heavy chain variable domain is the same for all the library members and thus the diversity is confined to the light chain variable domain. Such libraries are described, for example, WO 2010/135558. However, as the light chain variable domain is expressed in conjunction with the heavy variable domain, both domains can contribute to antigen binding. To further facilitate the process, antibody libraries containing the same heavy chain variable domain and either a diversity of Lambda variable light chains or Kappa variable light chains can be used in parallel for in vitro selection of antibodies against different antigens. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the invention. The bispecific antibodies of the invention can be of IgA Isotypes and their Fc portion can be modified in order to alter the bind properties to different Fc receptors and in this way modify the effectors functions of the antibody as well as it pharmacokinetic properties. Numerous methods for the modification of the Fc portion have been described and are applicable to antibodies of the invention, (see for example Strohl, W R Curr Opin Biotechnol 2009 (6):685-91).

Another key step of the invention is the optimization of co-expression of the common heavy chain and two different light chains into a single cell to allow for the assembly of a bispecific antibody of the invention. If all the polypeptides get expressed at the same level and get assembled equally well to form an immunoglobulin molecule then the ratio of monospecific (same light chains) and bispecific (two different light chains) should be 50%.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the invention. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

The co-expression of the three chains led to the assembly of three different antibodies: two monospecific and one bispecific antibodies. Their theoretical relative ratios should be 1:1:2 provided the expression levels and assembly rates are similar for both light chains. The bispecific antibodies were purified using a three-step affinity chromatography procedure: (1) Protein A: capture IgA (mono- and bi-), (2) Kappa select: capture IgA containing a Kappa light chain(s), and (3) Lambda select: capture IgG containing a Lambda light chain. Kappaselect and Lambdaselect are affinity chromatography media developed by BAC, BV and GE Healthcare.

The purified bispecific antibodies were characterized as follows. The flow-through and elution from each affinity purification step was analyzed by SDS-PAGE. The specificity and affinity of κλ-bodies was determined by ELISA and surface plasmon resonance. The methods of the invention allow for the identification of antibodies with affinities in the sub-nanomolar to nanomolar range without optimization. This is not obvious as the diversity in antibody libraries described herein is restricted to the light chain which contributes less to the binding energy in standard antibodies.

To avoid the requirement of having access to two antibodies having light chain variable domains of the Kappa and Lambda type being perceived as a limitation to the instant invention, the methods described herein allow for the generation of hybrid light chain in which a Lambda variable domain can be fused to a Kappa constant domain and conversely a Kappa variable domain can be fused to a Lambda constant domain. In some embodiments, the methods of generating bispecific and/or multi-specific antibodies use a complete serum-free chemically defined process. These methods incorporate the most widely used mammalian cell line in pharmaceutical industry, the Chinese Hamster Ovary (CHO) cell line. The methods described therein are used to generate both semi-stable and stable cell lines. The methods can be used to manufacture the bispecific and/or multi-specific antibodies of the invention at small scale (e.g., in an Erlenmeyer flask) and at mid-scale (e.g., in 25 L Wave bag). The methods are also readily adaptable for larger scale production of the bispecific and/or multi-specific antibodies, as well as antibody mixtures of the invention.

### Exemplary Antibody Constructs

In some embodiments, the CD47 binding region comprises a first heavy chain variable domain, comprising at least one, two, or three CDRs selected from (a) CDR-H1 comprising the amino acid sequence of SEQ ID NO: 4; (b) CDR-H2 comprising the amino acid sequence of SEQ ID NO: 5; (c) CDR-H3 comprising the amino acid sequence of SEQ ID NO: 6.

In some embodiments, the CD47 binding region comprises a first heavy chain variable domain, comprising at least one, two, or three CDRs selected from (a) CDR-H1 comprising the amino acid sequence of SEQ ID NO: 7; (b) CDR-H2 comprising the amino acid sequence of SEQ ID NO: 8; (c) CDR-H3 comprising the amino acid sequence of SEQ ID NO: 9.

In some embodiments, the CD47 binding region comprises a first light chain variable domain, comprising at least one, two, or three CDRs selected from (a) CDR-L1 comprising the amino acid sequence of SEQ ID NO: 13; (b) CDR-L2 comprising the amino acid sequence of SEQ ID NO: 14; and (c) CDR-L3 comprising the amino acid sequence of SEQ ID NO: 15.

In some embodiments, the CD47 binding region comprises a first light chain variable domain, comprising at least one, two, or three CDRs selected from (a) CDR-L1 comprising the amino acid sequence of SEQ ID NO: 16; (b) CDR-L2 comprising the amino acid sequence of SEQ ID NO: 17; and (c) CDR-L3 comprising the amino acid sequence of SEQ ID NO: 18.

In some embodiments, the CD47 binding region comprises a first heavy chain variable domain, comprising at least one, two, or three CDRs selected from (a) CDR-H1 comprising the amino acid sequence of SEQ ID NO: 4; (b) CDR-H2 comprising the amino acid sequence of SEQ ID NO: 5; (c) CDR-H3 comprising the amino acid sequence of SEQ ID NO: 6; and a first a first light chain variable domain, comprising at least one, two, or three CDRs selected from (a) CDR-L1 comprising the amino acid sequence of SEQ ID NO: 13; (b) CDR-L2 comprising the amino acid sequence of SEQ ID NO: 14; and (c) CDR-L3 comprising the amino acid sequence of SEQ ID NO: 15.

In some embodiments, the CD47 binding region comprises a first heavy chain variable domain, comprising at least one, two, or three CDRs selected from (a) CDR-H1 comprising the amino acid sequence of SEQ ID NO: 7; (b) CDR-H2 comprising the amino acid sequence of SEQ ID NO: 8; (c) CDR-H3 comprising the amino acid sequence of SEQ ID NO: 9, and a first light chain variable domain, comprising at least one, two, or three CDRs selected from (a) CDR-L1 comprising the amino acid sequence of SEQ ID NO: 16; (b) CDR-L2 comprising the amino acid sequence of SEQ ID NO: 17; and (c) CDR-L3 comprising the amino acid sequence of SEQ ID NO: 18.

In some embodiments, the CD47 binding region comprises a first heavy chain variable sequence having at least 90%, 91%, 92%, 93%, 94%, 95%, 96%, 97%, 98%, 99%, or 100% sequence identity to the amino acid sequence of SEQ ID NO: 23.

In some embodiments, a first heavy chain variable sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 23. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 23, including post-translational modifications of that sequence.

In some embodiments, the CD47 binding region comprises a first light chain variable domain (VL) having at least 90%, 91%, 92%, 93%, 94%, 95%, 96%, 97%, 98%, 99%, or 100% sequence identity to the amino acid sequence of SEQ ID NO: 26. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 26. In some embodiments, the substitutions, insertions, or deletions occur in regions outside the CDRs (e.g., in the FRs). Optionally, the antibody or antigen-binding fragment thereof comprises the VL sequence of SEQ ID NO: 26, including post-translational modifications of that sequence.

In some embodiments, the CD47 binding region comprises a first heavy chain variable domain amino acid sequence of SEQ ID NO: 23, and a first light chain variable domain amino acid sequence in SEQ ID NO: 26, including post-translational modifications of those sequences.

In some embodiments, the CD47 binding region comprises a first heavy chain variable sequence having at least 90%, 91%, 92%, 93%, 94%, 95%, 96%, 97%, 98%, 99%, or 100% sequence identity to the amino acid sequence of SEQ ID NO: 24.

In some embodiments, a first heavy chain variable sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 24. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 27, including post-translational modifications of that sequence.

In some embodiments, the CD47 binding region comprises a first light chain variable domain (VL) having at least 90%, 91%, 92%, 93%, 94%, 95%, 96%, 97%, 98%, 99%, or 100% sequence identity to the amino acid sequence of SEQ ID NO: 27. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 27. In some embodiments, the substitutions, insertions, or deletions occur in regions outside the CDRs (e.g., in the FRs). Optionally, the antibody or antigen-binding fragment thereof comprises the VL sequence of SEQ ID NO: 27, including post-translational modifications of that sequence.

In some embodiments, the CD47 binding region comprises a first heavy chain variable domain amino acid sequence of SEQ ID NO: 24, and a first light chain variable domain amino acid sequence in SEQ ID NO: 27, including post-translational modifications of those sequences.

In some embodiments, the antigen binding region comprises a second heavy chain variable domain, comprising at least one, two, or three CDRs selected from (a) CDR-H1 comprising the amino acid sequence of SEQ ID NO: 1; (b) CDR-H2 comprising the amino acid sequence of SEQ ID NO: 2; (c) CDR-H3 comprising the amino acid sequence of SEQ ID NO: 3.

In some embodiments, the antigen binding region comprises a second heavy chain variable domain, comprising at least one, two, or three CDRs selected from (a) CDR-H1 comprising the amino acid sequence of SEQ ID NO: 10; (b) CDR-H2 comprising the amino acid sequence of SEQ ID NO: 11; (c) CDR-H3 comprising the amino acid sequence of SEQ ID NO: 12.

In some embodiments, the antigen binding region comprises a second heavy chain variable domain, comprising at least one, two, or three CDRs selected from (a) CDR-H1 comprising the amino acid sequence of SEQ ID NO: 1; (b) CDR-H2 comprising the amino acid sequence of SEQ ID NO: 2; (c) CDR-H3 comprising the amino acid sequence of SEQ ID NO: 3; and a second a first light chain variable domain, comprising at least one, two, or three CDRs selected from (a) CDR-L1 comprising the amino acid sequence of SEQ ID NO: 10; (b) CDR-L2 comprising the amino acid sequence of SEQ ID NO: 11; and (c) CDR-L3 comprising the amino acid sequence of SEQ ID NO: 12.

In some embodiments, the antigen binding region comprises a second heavy chain variable domain,

In some embodiments, the antigen binding region comprises a second heavy chain variable sequence having at least 90%, 91%, 92%, 93%, 94%, 95%, 96%, 97%, 98%, 99%, or 100% sequence identity to the amino acid sequence of SEQ ID NO: 22.

In some embodiments, a second heavy chain variable sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in the amino acid sequence of SEQ ID NO: 22. In some embodiments, substitutions, insertions, or deletions occur in regions outside the CDRs (e.g., in the FRs). Optionally, the antibody or antigen-binding fragment thereof comprises the VH sequence of the amino acid sequence of SEQ ID NO: 22, including post-translational modifications of that sequence.

In some embodiments, the antigen binding region comprises a second light chain variable domain (VL) having at least 90%, 91%, 92%, 93%, 94%, 95%, 96%, 97%, 98%, 99%, or 100% sequence identity to the amino acid sequence of SEQ ID NO: 25. In some embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an antibody or antigen-binding fragment thereof comprising that sequence retains the ability to bind to antigen. In some embodiments, a total of 1 to 10 amino acids have been substituted, inserted and/or deleted in any one of the amino acid sequence of SEQ ID NO: 25. In some embodiments, the substitutions, insertions, or deletions occur in regions outside the CDRs (e.g., in the FRs). Optionally, the antibody or antigen-binding fragment thereof comprises the VL sequence of SEQ ID NO: 25, including post-translational modifications of that sequence.

In some embodiments, the antigen binding region comprises a second heavy chain variable domain amino acid sequence of SEQ ID NO: 22, and a second light chain variable domain amino acid sequence in SEQ ID NO: 25, including post-translational modifications of those sequences.

In some embodiments are constructs described herein wherein a CD47 binding region comprises CDRs from Table A, and an antigen binding region comprises CDRs from Table B. These CDRs and binding regions can be substituted with others known in the literature to achieve a desired binding profile to recruit an immune effector cell to a target antigen presenting cell.

### Exemplary DVD Ig Antibodies

In some embodiments, the antibody construct comprises a polypeptide, wherein the C-terminus of a second heavy chain variable domain is linked to the N-terminus of the first heavy chain variable domain. In some embodiments, the linking is via a linker peptide. In some embodiments, the linker peptide comprises an amino acid sequence that is at least at least 90%, 91%, 92%, 93%, 94%, 95%, 96%, 97%, 98%, 99%, or 100% identical to the amino acid sequence of SEQ ID NO: 44. In some embodiments, the polypeptide wherein the C-terminus of a second heavy chain variable domain is linked to the N-terminus of the first heavy chain variable domain, comprises an amino acid sequence that is at least 90%, 91%, 92%, 93%, 94%, 95%, 96%, 97%, 98%, 99%, or 100% identical to the amino acid sequence of SEQ ID NO: 29. In some embodiments, the C-terminus of the polypeptide is linked to an amino acid sequence encoding the IgA constant heavy chain.

In some embodiments, the antibody construct comprises a polypeptide, wherein the C-terminus of a second light chain variable domain is linked to the N-terminus of the first light chain variable domain. In some embodiments, the linking is via a linker peptide. In some embodiments, the linker peptide comprises an amino acid sequence that is at least at least 90%, 91%, 92%, 93%, 94%, 95%, 96%, 97%, 98%, 99%, or 100% identical to the amino acid sequence of SEQ ID NO: 44. In some embodiments, the polypeptide wherein the C-terminus of a second light chain variable domain is linked to the N-terminus of the first light chain variable domain, comprises an amino acid sequence that is at least 90%, 91%, 92%, 93%, 94%, 95%, 96%, 97%, 98%, 99%, or 100% identical to the amino acid sequence of SEQ ID NO: 31. In some embodiments, the C-terminus of the polypeptide is linked to an amino acid sequence encoding the IgA constant heavy chain.

In some embodiments, the bispecific antibody comprises a first polypeptide comprising an amino acid sequence that is at least 90%, 91%, 92%, 93%, 94%, 95%, 96%, 97%, 98%, 99%, or 100% identical to the amino acid sequence of SEQ ID NO: 29, and a second polypeptide comprising comprises an amino acid sequence that is at least 90%, 91%, 92%, 93%, 94%, 95%, 96%, 97%, 98%, 99%, or 100% identical to the amino acid sequence of SEQ ID NO: 31.

In some embodiments, the bispecific antibody comprises a first polypeptide comprising the C-terminus of the first heavy chain variable domain is linked to the N-terminus of the second heavy chain variable domain. In some embodiments, the linking is via a linker peptide. In some embodiments, the linker peptide comprises an amino acid sequence that is at least at least 90%, 91%, 92%, 93%, 94%, 95%, 96%, 97%, 98%, 99%, or 100% identical to the amino acid sequence of SEQ ID NO: 44. In some embodiments, the polypeptide comprising the C-terminus of the first heavy chain variable domain is linked to the N-terminus of the second heavy chain variable domain, comprises an amino acid sequence that is at least 90%, 91%, 92%, 93%, 94%, 95%, 96%, 97%, 98%, 99%, or 100% identical to the amino acid sequence of SEQ ID NO: 30. In some embodiments, the C-terminus of the polypeptide is linked to an amino acid sequence encoding the IgA constant heavy chain.

In some embodiments, the bispecific antibody comprises a first polypeptide comprising the C-terminus of the first light chain variable domain is linked to the N-terminus of the second light chain variable domain. In some embodiments, the linking is via a linker peptide. In some embodiments, the linker peptide comprises an amino acid sequence that is at least at least 90%, 91%, 92%, 93%, 94%, 95%, 96%, 97%, 98%, 99%, or 100% identical to the amino acid sequence of SEQ ID NO: 44. In some embodiments, the polypeptide comprising the C-terminus of the first light chain variable domain is linked to the N-terminus of the second light chain variable domain, comprises an amino acid sequence that is at least 90%, 91%, 92%, 93%, 94%, 95%, 96%, 97%, 98%, 99%, or 100% identical to the amino acid sequence of SEQ ID NO: 32. In some embodiments, the C-terminus of the polypeptide is linked to an amino acid sequence encoding the IgA constant heavy chain.

In some embodiments, the bispecific antibody comprises a first polypeptide comprising an amino acid sequence that is at least 90%, 91%, 92%, 93%, 94%, 95%, 96%, 97%, 98%, 99%, or 100% identical to the amino acid sequence of SEQ ID NO: 30, and a second polypeptide comprising comprises an amino acid sequence that is at least 90%, 91%, 92%, 93%, 94%, 95%, 96%, 97%, 98%, 99%, or 100% identical to the amino acid sequence of SEQ ID NO: 32.

In some embodiments, the antibody construct comprises a first polypeptide comprising a scFv linked to a light chain or heavy chain variable domain. In some embodiments, the linking is via a linker peptide. In some embodiments, the linker peptide comprises an amino acid sequence that is at least at least 90%, 91%, 92%, 93%, 94%, 95%, 96%, 97%, 98%, 99%, or 100% identical to the amino acid sequence of SEQ ID NO: 44 or SEQ ID NO: 45.

In some embodiments, the scFv comprises a second heavy chain variable domain and a second light chain variable domain, and is linked to a first light chain variable domain. In some embodiments, the first polypeptide comprising the scFv (e.g., comprising a second heavy chain variable domain and a second light chain variable domain), linked to a first light chain variable domain, comprises a sequence that is at least 90%, 91%, 92%, 93%, 94%, 95%, 96%, 97%, 98%, 99%, or 100% identical to the amino acid sequence of SEQ ID NO: 35.

In some embodiments, the scFv comprises a second heavy chain variable domain and a second light chain variable domain, and is linked to a first heavy chain variable domain. In some embodiments, the first polypeptide comprising the scFv (e.g., comprising a second heavy chain variable domain and a second light chain variable domain), linked to a first heavy chain variable domain, comprises a sequence that is at least 90%, 91%, 92%, 93%, 94%, 95%, 96%, 97%, 98%, 99%, or 100% identical to the amino acid sequence of SEQ ID NO: 33.

In some embodiments, the scFv comprises a first heavy chain variable domain and a first light chain variable domain, and is linked to a second light chain variable domain. In some embodiments, the first polypeptide comprising the scFv (e.g., comprising a first heavy chain variable domain and a first light chain variable domain), linked to a second light chain variable domain, comprises a sequence that is at least 90%, 91%, 92%, 93%, 94%, 95%, 96%, 97%, 98%, 99%, or 100% identical to the amino acid sequence of SEQ ID NO: 36.

In some embodiments, the scFv comprises a first heavy chain variable domain and a first light chain variable domain, and is linked to a second heavy chain variable domain. In some embodiments, the first polypeptide comprising the scFv (e.g., comprising a first heavy chain variable domain and a first light chain variable domain), linked to a second heavy chain variable domain, comprises a sequence that is at least 90%, 91%, 92%, 93%, 94%, 95%, 96%, 97%, 98%, 99%, or 100% identical to the amino acid sequence of SEQ ID NO: 34.

### Exemplary Kappa Lambda Bodies

In some embodiments, the antibody construct (e.g. a Kappa-lambda body) comprises a first light chain variable domain of the Kappa type or the lambda type, and a second light chain variable domain of the Kappa type or the Lambda type. In some embodiments, the antibody construct (e.g. a Kappa-lambda body) comprises a first light chain variable domain of the Kappa type and a second light chain variable domain of the Lambda type. In some embodiments, the antibody construct (e.g. a Kappa-lambda body) comprises a first light chain variable domain of the Lambda type and a second light chain variable domain of the Lambda type. In some embodiments, the CD47 binding region comprises a first light chain variable domain, comprising at least one, two, or three CDRs selected from (a) CDR-L1 comprising the amino acid sequence of SEQ ID NO: 16; (b) CDR-L2 comprising the amino acid sequence of SEQ ID NO: 17; and (c) CDR-L3 comprising the amino acid sequence of SEQ ID NO: 18. In some embodiments, the CD47 binding region comprises a first light chain variable domain (VL) having at least 90%, 91%, 92%, 93%, 94%, 95%, 96%, 97%, 98%, 99%, or 100% sequence identity to the amino acid sequence of SEQ ID NO: 27.

In some embodiments, the antigen binding domain comprises a second light chain variable domain, comprising at least one, two, or three CDRs selected from (a) CDR-L1 comprising the amino acid sequence of SEQ ID NO: 19; (b) CDR-L2 comprising the amino acid sequence of SEQ ID NO: 20; and (c) CDR-L3 comprising the amino acid sequence of SEQ ID NO: 21. In some embodiments, the antigen binding domain comprises a first light chain variable domain (VL) having at least 90%, 91%, 92%, 93%, 94%, 95%, 96%, 97%, 98%, 99%, or 100% sequence identity to the amino acid sequence of SEQ ID NO: 28.

In some embodiments, the first light chain variable domain further comprises a light chain constant domain. In some embodiments, the second light chain variable domain further comprises a light chain constant domain. In some embodiments, the light chain constant domain is of the Kappa type. In some embodiments, the light chain constant domain is of the lambda type. In some embodiments, the light chain constant domain is of the Kappa type comprising amino acid sequence having at least 90%, 91%, 92%, 93%, 94%, 95%, 96%, 97%, 98%, 99%, or 100% sequence identity to the amino acid sequence of SEQ ID NO: 42. In some embodiments, the light chain constant domain is of the Lambda type comprising amino acid sequence having at least 90%, 91%, 92%, 93%, 94%, 95%, 96%, 97%, 98%, 99%, or 100% sequence identity to the amino acid sequence of SEQ ID NO: 43.

In some embodiments, the antibody construct comprises a first heavy chain variable domain and a second heavy chain variable domain, wherein the first heavy chain variable domain and the second heavy chain variable domain is the same. In some embodiments, the first heavy chain variable domain and the second heavy chain variable domain comprises at least one, two, or three CDRs selected from (a) CDR-H1 comprising the amino acid sequence of SEQ ID NO: 7; (b) CDR-H2 comprising the amino acid sequence of SEQ ID NO: 8; (c) CDR-H3 comprising the amino acid sequence of SEQ ID NO: 9. In some embodiments, the first heavy chain variable domain and the second heavy chain variable domain comprises a sequence having at least 90%, 91%, 92%, 93%, 94%, 95%, 96%, 97%, 98%, 99%, or 100% sequence identity to the amino acid sequence of SEQ ID NO: 24.

In some embodiments, the antibody construct herein comprises a IgA heavy chain constant region. In some embodiments, the IgA heavy chain constant region comprises a sequence having at least 80%, 85%, 90%, 91%, 92%, 93%, 94%, 95%, 96%, 97%, 98%, 99%, or 100% sequence identity to the amino acid sequence of SEQ ID NO: 37. In some embodiments, the IgA heavy chain constant region comprises a sequence having at least 80%, 85%, 90%, 91%, 92%, 93%, 94%, 95%, 96%, 97%, 98%, 99%, or 100% sequence identity to the amino acid sequence of SEQ ID NO: 38. In some embodiments, the IgA heavy chain constant region comprises a sequence having at least 80%, 85%, 90%, 91%, 92%, 93%, 94%, 95%, 96%, 97%, 98%, 99%, or 100% sequence identity to the amino acid sequence of SEQ ID NO: 39. In some embodiments, the IgA heavy chain constant region comprises a sequence having at least 80%, 85%, 90%, 91%, 92%, 93%, 94%, 95%, 96%, 97%, 98%, 99%, or 100% sequence identity to the amino acid sequence of SEQ ID NO: 40. In some embodiments, the IgA heavy chain constant region comprises a sequence having at least 80%, 85%, 90%, 91%, 92%, 93%, 94%, 95%, 96%, 97%, 98%, 99%, or 100% sequence identity to the amino acid sequence of SEQ ID NO: 41.

### IgA Constant Region

In one embodiment, the bispecific IgA antibodies of the present disclosure contain a complete IgA heavy chain constant domain. In some embodiments, the IgA heavy chain constant domain comprise one or more modifications to create an asymmetric interface between two heavy chains

The term “asymmetric interface” is used to refer to an interface (as hereinabove defined) formed between two antibody chains, such as a first and a second IgA heavy chain constant region and/or between an IgA heavy chain constant region and its matching light chain, wherein the contact residues in the first and the second chains are different by design, comprising complementary contact residues. The asymmetric interface can be created by knobs/holes interactions and/or salt bridges coupling (charge swaps) and/or other techniques known in the art, such as for example, by the CrossMab approach for coupling an a heavy chain to its matching light chain. A “cavity” or “hole” refers to at least one amino acid side chain which is recessed from the interface of the second polypeptide and therefore accommodates a corresponding protuberance (“knob”) on the adjacent interface of the first polypeptide. The cavity (hole) may exist in the original interface or may be introduced synthetically (e.g. by altering nucleic acid encoding the interface). Normally, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. The upper limit for the number of original residues which are replaced is the total number of residues in the interface of the second polypeptide. The preferred import residues for the formation of a cavity are usually naturally occurring amino acid residues and are preferably selected from alanine (A), serine (S), threonine (T), valine (V) and glycine (G). Most preferred amino acid residues are serine, alanine or threonine, most preferably alanine. In the preferred embodiment, the original residue for the formation of the protuberance has a large side chain volume, such as tyrosine (Y), argmine (R), phenylalanine (F) or tryptophan (W).

An “original” amino acid residue is one which is replaced by an “import*’ residue which can have a smaller or larger side chain volume than the original residue. The import amino acid residue can be a naturally occurring or non-naturally occurring amino acid residue, but preferably is the former.

By “non-naturally occurring” amino acid residue is meant a residue which is not encoded by the genetic code, but which is able to covalently bind adjacent amino acid residue(s) in the polypeptide chain. Examples of non-naturally occurring amino acid residues are norleucine, ornithine, norvaline, homoserine and other amino acid residue analogues such as those described in Eilman et al Enzym. 202:301-336 (1991), for example. To generate such non-naturally occurring amino acid residues, the procedures of Noren et al. Science 244: 182 (1989) and Eilman et al., supra can be used. Briefly, this involves chemically activating a suppressor tRNA with a non-naturally occurring amino acid residue followed by in vitro transcription and translation of the RNA, The methods of the current invention, in certain embodiments, involve replacing at least one original amino acid residue in an IgM heavy chain, but more than one original residue can be replaced. Normally, no more than the total residues in the interface of the first or second polypeptide will comprise original amino acid residues which are replaced. The preferred original residues for replacement are “buried”, By “buried” is meant that the residue is essentially inaccessible to solvent. The preferred import residue is not cysteine to prevent possible oxidation or mispairing of di sulfide bonds. The protuberance is “positionable” in the cavity which means that the spatial location of the protuberance and cavity on the interface of the first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe and Trp do not typically extend perpendicularly from the x is of the interface and. have preferred, conformations, the alignment of a protuberance with a corresponding cavity relies on modeling the protuberance/cavity pair based upon a three-dimensional structure such as that obtained by X-ray crystallography or nuclear magnetic resonance (NMR). This can be achieved using widely accepted techniques in the art, including techniques of molecular modeling,

By “original nucleic acid” is meant the nucleic acid encoding a polypeptide of interest, which can be “altered” (i.e. genetically engineered or mutated) to encode a protuberance or cavity. The original or starting nucleic acid may be a naturally occurring nucleic acid or may comprise a nucleic acid which has been subjected to prior alteration (e.g. a humanized antibody fragment). By “altering” the nucleic acid is meant that the original nucleic acid is mutated by inserting, deleting or replacing at least one codon encoding an amino acid residue of interest. Normally, a codon encoding an original residue is replaced by a codon encoding an import residue. Techniques for genetically modifying a DNA in this manner have been reviewed in Mutagenesis: a Practical Approach. M, J. McPherson, Ed. (IRL Press, Oxford. UK. (1991), and include site-directed mutagenesis, cassette mutagenesis and polymerase chain reaction (PGR) mutagenesis, for example.

The protuberance or cavity can be “introduced” into the interface of the first or second polypeptide by synthetic means, e.g. by recombinant techniques, in vitro peptide synthesis, those techniques for introducing non-naturally occurring amino acid residues previously described, by enzymatic or chemical coupling of peptides or some combination of these techniques. According, the protuberance or cavity which is “introduced” is “non-naturally occurring” or “non-native”, which means that it does not exist in nature or in the original polypeptide (e.g. a humanized monoclonal antibody).

Preferably the import amino acid residue for forming the protuberance has a relatively small number of “rotamers” (e.g. about 3-6). A “rotamer” is an. energetically favorable conformation of an amino acid side chain. The number of rotamers for the various amino acid residue

In a further embodiment, the multi-specific IgA antibodies of the present invention comprise a complete native J chain. The J chain Is a key protein in. the generation of SlgA because it promotes polymerization of IgA and because its presence in these polymers is believed to be required for their affinity to SC/plgR. The multi-specific IgA antibodies herein may comprise a I chain fragment, or an. otherwise modified J chain, as long as the fragment or modified J chain retains the function of native J chain, in particular to enable efficient polymerization of IgA and binding of such polymers to the secretory component (SC)/polymeric (p)IgR-For further details of the structure-function relationship of J chain see. e.g. Johansen et a)., 2001; j. Immunol, 167(9):5185-5192.

In order to generate an IgA molecule with two different a heavy chains (i.e. where at least one of the binding nnlts is bispecific), a solution must be found for coupling the two matching a heavy chains with two different binding specificities to each other. In addition, if a light chain is needed to form a binding region, a solution must be found to couple each heavy chain with its matching light chain to provide the desired binding specificity.

The coupling can be achieved by salt bridge pairs charge switching (also referred to as charge swaps or charge reversals) between certain residues and/or by creating knobs-holes interactions between the two chains. The heavy chains can also be paired with their matching light chains by using the CrossMab technique. The different approaches can also be combined in order to achieve an optimal result.

### Knobs-into-Holes Technique

To improve the yields of the penta- or hexameric bispecific binding molecules of the present invention, the IgA heavy chain constant regions, e.g. the CH3 domains, can be altered by the “knob-into-holes” technique which is described in detail with several examples in e.g. WO 96/02701 1, Ridgway, J., B., et al., Protein Eng 9 (1996) 617-621; and Merchant, A. M, et ah, Nat Bioiechno). 16 (1998) 677-681. In this method the interaction surfaces of two IgA heavy chain constant domains are altered to increase the heterodimerization of two heavy chains with different binding specificities and/or between a heavy chain and its matching light chain. Each of the two heavy chain domains, can be the “knobe” while the other is the “hole”. The introduction of a disulfide bridge stabilizes the heterodimers (Merchant, A. M.s et ah, Nature Biotech 16 (1998) 677-681; Atwell, S., et al., J. Moi. Biol. 270 (1997) 26-35) and increases the yield. Similarly, the matching heavy and light chains can be coupled to each other by this technique (Zhu, Z.; Presta, L. G.; Zapata, G.; Carter, P. Remodeling domain interfaces to enhance heterodimer formation. Prof Sci. 6:781-788 (1997)).

Following this approach, within the original interface of the Co. (e.g. Ca3) domains of one heavy chain that meets the original interface of the corresponding domain of the other heavy chain within the bispecific IgA antibody, an amino acid residue may be replaced with an amino acid residue having a larger side chain volume, thereby creating a protuberance within the interface, which is positionable in a cavity within the interface of the corresponding domain in the other IgA heavy chain constant region. Similarly, the second IgA heavy chain may be altered, by replacing an amino acid residue within the interface with a corresponding domain in the constant region of the first IgA heavy chain, with an amino acid residue having a smaller side chain volume, thereby creating a hole (cavity) within the interface between the two heavy chain regions.

### Salt Bridge Pairs Charge Switching (Charge Swapping)

Opposite charges attract and similar charges repel each other, The charge of an amino acid molecule is pH dependent and can be characterized by the pK values, which are determined for the alpha amino group (N), the alpha carboxy group (C) and the side chain for free amino acids. The local environment can alter the pH of a side chain when the amino acid is part of a protein or peptide.

The charge properties of an amino acid molecule can also be characterized by the isoelectric point (pi), which is the pH at which the overall charge of the molecule is neutral. Since amino acids differ from each other in their side chains, the pi reflects differences in the pKs of the side chains.

Most amino acids (15 out of 20) have a pi close to 6 so they are regarded as having neutral overall charge. Asp and Glu are negatively charged, and His, Lys, Arg are positively charged.

One or more of these mutations, or sets of mutations, can be combined with one or more sets of knobs-holes mutations to provide a desired asymmetric interface between two different IgA heavy chains and/or between an IgA heavy chain and its matching light chain.

Preferably, the asymmetric interface between two different IgA heavy chain constant regions is created by up to 8, such as, for example, 1-8, or 1-7, or 1-6, or 1-5, or 1-4, or 1-3, or 1-2 mutations in one IgA heavy chain, or 2-10, or 2-9, or 2-8, or 2-7, or 2-6, or 2-5, or 2-4, or 2-3 combined mutations in the two IgA heavy chains.

### CrossMab Technique

As discussed above, the knobs-into-holes technology or charge swapping enables heterodimerization of the antibody heavy chains. Correct association of the light chains and their cognate heavy chains can be achieved by exchange of heavy-chain and light-chain domains within the antigen binding fragment (Fab) of one half of the bispecific antibody binding unit Crossover can occur as a crossover of the complete VH-CH and VL-CL domains, crossover of only the VH and VL domains, or the CA and CL domains within the one half of the bispecific binding unit of an igA antibody. This “crossover’ retains the antigen-binding affinity but makes the two arms so different that light-chain mispairing can no longer occur. For further details, in the context of IgG antibodies, see, for example, Sehaeffer et al., (2011) Proc Natl Acad Sci USA 108(27): 1 1 187-11192.

### Immunoconjugates

In certain embodiments, the antibodies or fragments thereof may be conjugated to one or more therapeutic or diagnostic agents. The therapeutic agents do not need to be the same but can be different, e.g. a drug and a radioisotope. For example, 131I can be incorporated into a tyrosine of an antibody or fusion protein and a drug attached to an epsilon amino group of a lysine residue. Therapeutic and diagnostic agents also can be attached, for example to reduced SH groups and/or to carbohydrate side chains. Many methods for making covalent or non-covalent conjugates of therapeutic or diagnostic agents with antibodies or fusion proteins are known in the art and any such known method may be utilized.

A therapeutic or diagnostic agent can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56: 244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995). Alternatively, the therapeutic or diagnostic agent can be conjugated via a carbohydrate moiety in the Fc region of the antibody. The carbohydrate group can be used to increase the loading of the same agent that is bound to a thiol group, or the carbohydrate moiety can be used to bind a different therapeutic or diagnostic agent.

Methods for conjugating peptides to antibody components via an antibody carbohydrate moiety are well-known to those of skill in the art. See, for example, Shih et al., Int. J. Cancer 41: 832 (1988); Shih et al., Int. J. Cancer 46: 1101 (1990); and Shih et al., U.S. Pat. No. 5,057,313, incorporated herein in their entirety by reference. The general method involves reacting an antibody component having an oxidized carbohydrate portion with a carrier polymer that has at least one free amine function. This reaction results in an initial Schiff base (imine) linkage, which can be stabilized by reduction to a secondary amine to form the final conjugate.

The Fc region may be absent if the antibody used as the antibody component of the immunoconjugate is an antibody fragment. However, it is possible to introduce a carbohydrate moiety into the light chain variable region of a full length antibody or antibody fragment. See, for example, Leung et al., J. Immunol. 154: 5919 (1995); Hansen et al., U.S. Pat. No. 5,443,953 (1995), Leung et al., U.S. Pat. No. 6,254,868, incorporated herein by reference in their entirety. The engineered carbohydrate moiety is used to attach the therapeutic or diagnostic agent.

In some embodiments, a chelating agent may be attached to an antibody, antibody fragment or fusion protein and used to chelate a therapeutic or diagnostic agent, such as a radionuclide. Exemplary chelators include but are not limited to DTPA (such as Mx-DTPA), DOTA, TETA, NETA or NOTA. Methods of conjugation and use of chelating agents to attach metals or other ligands to proteins are well known in the art (see, e.g., U.S. Pat. No. 7,563,433, the Examples section of which is incorporated herein by reference).

In certain embodiments, radioactive metals or paramagnetic ions may be attached to proteins or peptides by reaction with a reagent having a long tail, to which may be attached a multiplicity of chelating groups for binding ions. Such a tail can be a polymer such as a polylysine, polysaccharide, or other derivatized or derivatizable chains having pendant groups to which can be bound chelating groups such as, e.g., ethylenediaminetetraacetic acid (EDTA), diethylenetriaminepentaacetic acid (DTPA), porphyrins, polyamines, crown ethers, bis-thiosemicarbazones, polyoximes, and like groups known to be useful for this purpose.

Chelates may be directly linked to antibodies or peptides, for example as disclosed in U.S. Pat. No. 4,824,659, incorporated herein in its entirety by reference. Particularly useful metal-chelate combinations include 2-benzyl-DTPA and its monomethyl and cyclohexyl analogs, used with diagnostic isotopes in the general energy range of 60 to 4,000 keV, such as 125I, 131I, 123I, 124I, 62Cu, 64Cu, 18F, 111In, 67Ga, 68Ga, 99mTc, 94mTc, 11C, 13N, 150, 76Br, for radioimaging. The same chelates, when complexed with non-radioactive metals, such as manganese, iron and gadolinium are useful for MM. Macrocyclic chelates such as NOTA, DOTA, and TETA are of use with a variety of metals and radiometals, most particularly with radionuclides of gallium, yttrium and copper, respectively. Such metal-chelate complexes can be made very stable by tailoring the ring size to the metal of interest. Other ring-type chelates such as macrocyclic polyethers, which are of interest for stably binding nuclides, such as 223Ra for RAIT are encompassed.

More recently, methods of 18F-labeling of use in PET scanning techniques have been disclosed, for example by reaction of F-18 with a metal or other atom, such as aluminum. The 18F—Al conjugate may be complexed with chelating groups, such as DOTA, NOTA or NETA that are attached directly to antibodies or used to label targetable constructs in pre-targeting methods. Such F-18 labeling techniques are disclosed in U.S. Pat. No. 7,563,433, the Examples section of which is incorporated herein by reference.

Another exemplary immunoconjugate was disclosed in Johannson et al. (2006, AIDS 20:1911-15), in which a doxorubicin-conjugated P4/D10 (anti-gp120) antibody was found to be highly efficacious in treating cells infected with HIV.

### Additional Therapeutic Agents

In alternative embodiments, therapeutic agents such as cytotoxic agents, anti-angiogenic agents, pro-apoptotic agents, antibiotics, hormones, hormone antagonists, chemokines, drugs, prodrugs, toxins, enzymes or other agents may be used, either conjugated to the subject bsAbs, ADCs and/or antibodies or separately administered before, simultaneously with, or after the bsAbs, ADCs and/or antibodies. Drugs of use may possess a pharmaceutical property selected from the group consisting of antimitotic, antikinase, alkylating, antimetabolite, antibiotic, alkaloid, anti-angiogenic, pro-apoptotic agents and combinations thereof.

Exemplary drugs of use may include, but are not limited to, 5-fluorouracil, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, camptothecin, carboplatin, 10-hydroxycamptothecin, carmustine, celebrex, chlorambucil, cisplatin (CDDP), Cox-2 inhibitors, irinotecan (CPT-11), SN-38, carboplatin, cladribine, camptothecans, crizotinib, cyclophosphamide, cytarabine, dacarbazine, dasatinib, dinaciclib, docetaxel, dactinomycin, daunorubicin, doxorubicin, 2-pyrrolinodoxorubicine (2P-DOX), cyano-morpholino doxorubicin, doxorubicin glucuronide, epirubicin glucuronide, erlotinib, estramustine, epidophyllotoxin, erlotinib, entinostat, estrogen receptor binding agents, etoposide (VP16), etoposide glucuronide, etoposide phosphate, exemestane, fingolimod, floxuridine (FUdR), 3′,5′-O-dioleoyl-FudR (FUdR-dO), fludarabine, flutamide, farnesyl-protein transferase inhibitors, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechlorethamine, melphalan, mercaptopurine, 6-mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide (an aqueous form of DTIC), transplatinum, thalidomide, thioguanine, thiotepa, teniposide, topotecan, uracil mustard, vatalanib, vinorelbine, vinblastine, vincristine, vinca alkaloids and ZD1839.

Toxins of use may include ricin, abrin, alpha toxin, saporin, ribonuclease (RNase), e.g., onconase, DNase I, Staphylococcal enterotoxin-A, pokeweed antiviral protein, gelonin, diphtheria toxin, Pseudomonas exotoxin, and Pseudomonas endotoxin.

Chemokines of use may include RANTES, MCAF, MIP1-alpha, MIP1-Beta and IP-10. In certain embodiments, anti-angiogenic agents, such as angiostatin, baculostatin, canstatin, maspin, anti-VEGF antibodies, anti-P1GF peptides and antibodies, anti-vascular growth factor antibodies, anti-Flk-1 antibodies, anti-Flt-1 antibodies and peptides, anti-Kras antibodies, anti-cMET antibodies, anti-MIF (macrophage migration-inhibitory factor) antibodies, laminin peptides, fibronectin peptides, plasminogen activator inhibitors, tissue metalloproteinase inhibitors, interferons, interleukin-12, IP-10, Gro-β, thrombospondin, 2-methoxyoestradiol, proliferin-related protein, carboxiamidotriazole, CM101, Marimastat, pentosan polysulphate, angiopoietin-2, interferon-alpha, herbimycin A, PNU145156E, 16K prolactin fragment, Linomide (roquinimex), thalidomide, pentoxifylline, genistein, TNP-470, endostatin, paclitaxel, accutin, angiostatin, cidofovir, vincristine, bleomycin, AGM-1470, platelet factor 4 or minocycline may be of use.

Immunomodulators of use may be selected from a cytokine, a stem cell growth factor, a lymphotoxin, a hematopoietic factor, a colony stimulating factor (CSF), an interferon (IFN), erythropoietin, thrombopoietin and a combination thereof. Specifically useful are lymphotoxins such as tumor necrosis factor (TNF), hematopoietic factors, such as interleukin (IL), colony stimulating factor, such as granulocyte-colony stimulating factor (G-CSF) or granulocyte macrophage-colony stimulating factor (GM-CSF), interferon, such as interferons-α, -β or -λ and stem cell growth factor, such as that designated “51 factor”. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and —II; erythropoietin (EPO); osteoinductive factors; interferons such as interferon-α, -β, and -γ; colony stimulating factors (CSFs) such as macrophage-CSF (M-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21, IL-25, LIF, kit-ligand or FLT-3, angiostatin, thrombospondin, endostatin, tumor necrosis factor and LT.

Radionuclides of use include, but are not limited to—111In, 177Lu, 212Bi, 213Bi, 211At, 62Cu, 67Cu, 90Y, 125I, 131I, 32P, 33P, 47Sc, 111Ag, 67G a, 142Pr, 153Sm, 161Tb, 166Dy, 166Ho, 186Re, 188Re, 189Re, 212Pb, 223Ra, 225Ac, 59Fe, 75Se, 77As, 89Sr, 99Mo, 105Rh, 109Pd, 143Pr, 149Pm, 169Er, 194Ir, 198Au, 199Au, 211Pb, and 227Th. The therapeutic radionuclide preferably has a decay-energy in the range of 20 to 6,000 keV, preferably in the ranges 60 to 200 keV for an Auger emitter, 100-2,500 keV for a beta emitter, and 4,000-6,000 keV for an alpha emitter. Maximum decay energies of useful beta-particle-emitting nuclides are preferably 20-5,000 keV, more preferably 100-4,000 keV, and most preferably 500-2,500 keV. Also preferred are radionuclides that substantially decay with Auger-emitting particles. For example, Co-58, Ga-67, Br-80m, Tc-99m, Rh-103m, Pt-109, In-111, Sb-119, 1-125, Ho-161, Os-189m and Ir-192. Decay energies of useful beta-particle-emitting nuclides are preferably <1,000 keV, more preferably <100 keV, and most preferably <70 keV. Also preferred are radionuclides that substantially decay with generation of alpha-particles. Such radionuclides include, but are not limited to: Dy-152, At-211, Bi-212, Ra-223, Rn-219, Po-215, Bi-211, Ac-225, Fr-221, At-217, Bi-213, Th-227 and Fm-255. Decay energies of useful alpha-particle-emitting radionuclides are preferably 2,000-10,000 keV, more preferably 3,000-8,000 keV, and most preferably 4,000-7,000 keV. Additional potential radioisotopes of use

include 11C, 13N, 15O, 75Br, 198Au, 224Ac, 1261, 1331, 77Br, 113mIn, 95Ru, 97Ru, 103Ru, 105Ru, 107Hg, 203Hg, 121mTe, 122mTe, 125mTe, 165Tm, 167Tm, 168Tm, 197Pt, 109Pd, 105 Rb, 142Pr, 143Pr, 161Tb, 166Ho, 199Au, 57Co, 58Co, 51Cr, 59Fe, 75Se, 201T1, 225Ac, 76Br, 169Y b, and the like. Some useful diagnostic nuclides may
include 18F, 52Fe, 62Cu, 64Cu, 67Cu, 67Ga, 68Ga, 86Y, 89Zr, 94Tc, 94mTc, 99mTc, or, 111In.

Therapeutic agents may include a photoactive agent or dye. Fluorescent compositions, such as fluorochrome, and other chromogens, or dyes, such as porphyrins sensitive to visible light, have been used to detect and to treat lesions by directing the suitable light to the lesion. In therapy, this has been termed photoradiation, phototherapy, or photodynamic therapy. See Joni et al. (eds.), PHOTODYNAMIC THERAPY OF TUMORS AND OTHER DISEASES (Libreria Progetto 1985); van den Bergh, Chem. Britain (1986), 22:430. Moreover, monoclonal antibodies have been coupled with photoactivated dyes for achieving phototherapy. See Mew et al., J. Immunol. (1983), 130:1473; idem., Cancer Res. (1985), 45:4380; Oseroff et al., Proc. Natl. Acad. Sci. USA (1986), 83:8744; idem., Photochem. Photobiol. (1987), 46:83; Hasan et al., Prog. Clin. Biol. Res. (1989), 288:471; Tatsuta et al., Lasers Surg. Med. (1989), 9:422; Pelegrin et al., Cancer (1991), 67:2529.

Other useful therapeutic agents may comprise oligonucleotides, especially antisense oligonucleotides that preferably are directed against oncogenes and oncogene products, such as bcl-2 or p53. A preferred form of therapeutic oligonucleotide is siRNA. The skilled artisan will realize that any siRNA or interference RNA species may be attached to an antibody or fragment thereof for delivery to a targeted tissue. Many siRNA species against a wide variety of targets are known in the art, and any such known siRNA may be utilized in the claimed methods and compositions.

Known siRNA species of potential use include those specific for IKK-gamma (U.S. Pat. No. 7,022,828); VEGF, Flt-1 and Flk-1/KDR (U.S. Pat. No. 7,148,342); Bcl2 and EGFR (U.S. Pat. No. 7,541,453); CDC20 (U.S. Pat. No. 7,550,572); transducin (beta)-like 3 (U.S. Pat. No. 7,576,196); KRAS (U.S. Pat. No. 7,576,197); carbonic anhydrase II (U.S. Pat. No. 7,579,457); complement component 3 (U.S. Pat. No. 7,582,746); interleukin-1 receptor-associated kinase 4 (IRAK4) (U.S. Pat. No. 7,592,443); survivin (U.S. Pat. No. 7,608,7070); superoxide dismutase 1 (U.S. Pat. No. 7,632,938); MET proto-oncogene (U.S. Pat. No. 7,632,939); amyloid beta precursor protein (APP) (U.S. Pat. No. 7,635,771); IGF-1R (U.S. Pat. No. 7,638,621); ICAM1 (U.S. Pat. No. 7,642,349); complement factor B (U.S. Pat. No. 7,696,344); p53 (U.S. Pat. No. 7,781,575), and apolipoprotein B (U.S. Pat. No. 7,795,421), the Examples section of each referenced patent incorporated herein by reference.

Additional siRNA species are available from known commercial sources, such as Sigma-Aldrich (St Louis, Mo.), Invitrogen (Carlsbad, Calif.), Santa Cruz Biotechnology (Santa Cruz, Calif.), Ambion (Austin, Tex.), Dharmacon (Thermo Scientific, Lafayette, Colo.), Promega (Madison, Wis.), Minis Bio (Madison, Wis.) and Qiagen (Valencia, Calif.), among many others. Other publicly available sources of siRNA species include the siRNAdb database at the Stockholm Bioinformatics Centre, the MIT/ICBP siRNA Database, the RNAi Consortium shRNA Library at the Broad Institute, and the Probe database at NCBI. For example, there are 30,852 siRNA species in the NCBI Probe database. The skilled artisan will realize that for any gene of interest, either a siRNA species has already been designed, or one may readily be designed using publicly available software tools. Any such siRNA species may be delivered using the subject DNL® complexes.

### Methods of Treatment

Disclosed herein, in certain embodiments, are methods of treating a subject in need thereof, comprising administering to the subject a therapeutic dose of the therapeutic agents or pharmaceutical compositions disclosed herein. In some examples, the subject has a cancer or an infectious disease. In some cases, the cancer is a cancer associated with an expression of CD20, GD2, CD38, CD19, EGFR, HER2, PD-L1, CD25, CD33, BCMA, CD44, α-Folate receptor, CAIX, CD30, ROR1, CEA, EGP-2, EGP-40, HER3, Folate-binding Protein, GD3, IL-13R-a2, KDR, EDB-F, mesothelin, CD22, EGFR, MUC-1, MAGE-A1, MUC16, h5T4, PSMA, TAG-72, EGFRvIII, CD123 or VEGF-R2.

In some embodiments, the pharmaceutical composition or the therapeutic agent is administered intravenously, cutaneously, subcutaneously, or injected at a site of an affliction. In certain cases, the pharmaceutical composition or the therapeutic agent is administered as a single dose or in divided doses within about 48 hours of each other. In some examples, the pharmaceutical composition or the therapeutic agent induces greater immune activation against a cancer as measured by a decrease in cancer cell number or volume as compared to non-cancerous tissue.

In some embodiments, disclosed herein are methods of administering a therapeutic agent or pharmaceutical composition described herein, to a subject having a cancer associated with an overexpression of CD20. In some embodiments, disclosed herein are methods of administering a therapeutic agent or pharmaceutical composition described herein to a subject having a cancer associated with an overexpression of GD2. In some embodiments, disclosed herein are methods of administering a therapeutic agent or pharmaceutical composition described herein to a subject having a cancer associated with an overexpression of mesothelin. In some embodiments, disclosed herein are methods of administering a modified effector cell to a subject having a cancer associated with an overexpression of CD38, CD19, EGFR, HER2, PD-L1, CD25, CD33, BCMA, CD44, α-Folate receptor, CAIX, CD30, ROR1, CEA, EGP-2, EGP-40, HER3, Folate-binding Protein, GD2, GD3, IL-13R-a2, KDR, EDB-F, mesothelin, CD22, EGFR, MUC-1, MAGE-A1, MUC16, h5T4, PSMA, TAG-72, EGFRvIII, CD123 or VEGF-R2.

In some cases, the cancer is a metastatic cancer. In other cases, the cancer is a relapsed or refractory cancer.

In some cases, a cancer is a solid tumor or a hematologic malignancy. In some instances, the cancer is a solid tumor. In other instances, the cancer is a hematologic malignancy. In some cases, the cancer is a metastatic cancer. In some cases, the cancer is a relapsed or refractory cancer.

In some instances, the cancer is a solid tumor. Exemplary solid tumors include, but are not limited to, anal cancer; appendix cancer; bile duct cancer (i.e., cholangiocarcinoma); bladder cancer; brain tumor; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; vulvar cancer; or glioblastoma.

“Glioblastoma” or “glioblastoma multiforme” (GBM) is an aggressive neuroepithelial brain cancer. GBM may originate from glial type cells, astrocytes, oligodendrocyte progenitor cells, or neural stem cells. Four subtypes of glioblastoma have been identified. The classical subtype, a majority of GBM, carries extra copies of the epidermal growth factor receptor (EGFR) gene, and most have higher than normal expression of epidermal growth factor receptor (EGFR). In a subset of the cases, EGFR amplification is accompanied by gene rearrangement, the most common of which is EGFR variant III (EGFRvIII). The gene TP53 (p53), which is often mutated in glioblastoma, is rarely mutated in the classical subtype. The proneural subtype often has high rates of alterations in TP53 (p53), and in PDGFRA, the gene encoding platelet-derived growth factor receptor A, and in IDH1, the gene encoding isocitrate dehydrogenase-1. The Mesenchymal subtype is characterized by high rates of mutations or other alterations in NF1, the gene encoding neurofibromin 1 and fewer alterations in the EGFR gene and less expression of EGFR than other types. The Neural subtype was typified by the expression of neuron markers such as NEFL, GABRA1, SYT1 and SLC12A5. Other genetic alterations have been described in glioblastoma, and the majority of them are clustered in two pathways, the RB and the PI3K/AKT. Glioblastomas have alterations in 68-78% and 88% of these pathways, respectively.

In some instances, the cancer is a hematologic malignancy. In some cases, a hematologic malignancy comprises a lymphoma, a leukemia, a myeloma, or a B-cell malignancy. In some cases, a hematologic malignancy comprises a lymphoma, a leukemia or a myeloma. In some instances, exemplary hematologic malignancies include chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic malignancy comprises a myeloid leukemia. In some embodiments, the hematologic malignancy comprises acute myeloid leukemia (AML) or chronic myeloid leukemia (CML).

In some instances, disclosed herein are methods of administering to a subject having a hematologic malignancy selected from chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis a modified effector cell described herein. In some instances, disclosed herein are methods of administering to a subject having a hematologic malignancy selected from AML or CML a modified effector cell to the subject.

In other cases, disclosed herein are methods of administering to a subject having an infection due to an infectious disease. An infectious disease can be a disease resulting from a bacterial, viral or fungi infection. In other instances, exemplary viral pathogens include those of the families of Adenoviridae, Epstein-Barr virus (EBV), Cytomegalovirus (CMV), Respiratory Syncytial Virus (RSV), JC virus, BK virus, HSV, HHV family of viruses, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. Exemplary pathogenic viruses cause smallpox, influenza, mumps, measles, chickenpox, ebola, and rubella. Exemplary pathogenic fungi include Candida, Aspergillus, Cryptococcus, Histoplasma, Pneumocystis, and Stachybotrys. Exemplary pathogenic bacteria include Streptococcus, Pseudomonas, Shigella, Campylobacter, Staphylococcus, Helicobacter, E. coli, Rickettsia, Bacillus, Bordetella, Chlamydia, Spirochetes, and Salmonella.

### Use of Bispecific Antibodies

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the invention, which include an antibody of the invention, are used to treat or alleviate a symptom associated with a cancer, such as, by way of non-limiting example, leukemias, lymphomas, breast cancer, colon cancer, ovarian cancer, bladder cancer, prostate cancer, glioma, lung & bronchial cancer, colorectal cancer, pancreatic cancer, esophageal cancer, liver cancer, urinary bladder cancer, kidney and renal pelvis cancer, oral cavity & pharynx cancer, uterine corpus cancer, and/or melanoma The present invention also provides methods of treating or alleviating a symptom associated with a cancer. A therapeutic regimen is carried out by identifying a subject, e.g., a human patient suffering from (or at risk of developing) a cancer, using standard methods.

Efficaciousness of treatment is determined in association with any known method for diagnosing or treating the particular immune-related disorder. Alleviation of one or more symptoms of the immune-related disorder indicates that the antibody confers a clinical benefit.

Methods for the screening of antibodies that possess the desired specificity include, but are not limited to, enzyme linked immunosorbent assay (ELISA) and other immunologically mediated techniques known within the art.

Antibodies directed against a target such as CD47, a tumor associated antigen or other antigen (or a fragment thereof) may be used in methods known within the art relating to the localization and/or quantitation of these targets, e.g., for use in measuring levels of these targets within appropriate physiological samples, for use in diagnostic methods, for use in imaging the protein, and the like). In a given embodiment, antibodies specific any of these targets, or derivative, fragment, analog or homolog thereof, that contain the antibody derived antigen binding domain, are utilized as pharmacologically active compounds (referred to hereinafter as “Therapeutics”).

An antibody of the invention can be used to isolate a particular target using standard techniques, such as immunoaffinity, chromatography or immunoprecipitation. Antibodies of the invention (or a fragment thereof) can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Antibodies of the invention, including polyclonal, monoclonal, humanized and fully human antibodies, may be used as therapeutic agents. Such agents will generally be employed to treat or prevent a disease or pathology associated with aberrant expression or activation of a given target in a subject. An antibody preparation, preferably one having high specificity and high affinity for its target antigen, is administered to the subject and will generally have an effect due to its binding with the target. Administration of the antibody may abrogate or inhibit or interfere with the signaling function of the target. Administration of the antibody may abrogate or inhibit or interfere with the binding of the target with an endogenous ligand to which it naturally binds. For example, the antibody binds to the target and neutralizes or otherwise inhibits the interaction between CD47 and SIRPα.

A therapeutically effective amount of an antibody of the invention relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies or a fragment thereof of the invention can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of a given target (or a protein fragment thereof) in a sample. In some embodiments, the antibody contains a detectable label. Antibodies are polyclonal, or more preferably, monoclonal. An intact antibody, or a fragment thereof (e.g., Fab, scFv, or F(ab)2 is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Pharmaceutical Compositions and Dosage

Disclosed herein, in certain embodiments, are pharmaceutical compositions comprising a therapeutic agent disclosed herein for administration in a subject.

In some instances, pharmaceutical compositions comprising a therapeutic agent described herein are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some instances, the pharmaceutical composition is formulated for parenteral (e.g., intravenous, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Proliferative disease” as referred to herein means a unifying concept that excessive proliferation of cells and turnover of cellular matrix contribute significantly to the pathogenesis of several diseases, including cancer is presented.

“Patient” or “subject” as used herein refers to a mammalian subject diagnosed with or suspected of having or developing a physiological condition, for instance a cancer or an autoimmune condition or an infection. In some embodiments, the term “patient” refers to a mammalian subject with a higher than average likelihood of developing cancer. Exemplary patients may be humans, apes, dogs, pigs, cattle, cats, horses, goats, sheep, rodents and other mammalians that can benefit from the therapies disclosed herein. Exemplary human patients can be male and/or female.

“Patient in need thereof” or “subject in need thereof” is referred to herein as a patient diagnosed with or suspected of having a disease or disorder, for instance, but not restricted to a proliferative disorder such as cancer. In some cases, a cancer is a solid tumor or a hematologic malignancy. In some instances, the cancer is a solid tumor. In other instances, the cancer is a hematologic malignancy. In some cases, the cancer is a metastatic cancer. In some cases, the cancer is a relapsed or refractory cancer. In some instances, the cancer is a solid tumor. Exemplary solid tumors include, but are not limited to, anal cancer; appendix cancer; bile duct cancer (i.e., cholangiocarcinoma); bladder cancer; brain tumor; breast cancer; cervical cancer; colon cancer; cancer of Unknown Primary (CUP); esophageal cancer; eye cancer; fallopian tube cancer; gastroenterological cancer; kidney cancer; liver cancer; lung cancer; medulloblastoma; melanoma; oral cancer; ovarian cancer; pancreatic cancer; parathyroid disease; penile cancer; pituitary tumor; prostate cancer; rectal cancer; skin cancer; stomach cancer; testicular cancer; throat cancer; thyroid cancer; uterine cancer; vaginal cancer; vulvar cancer; or glioblastoma. In some embodiments leukemia can be, for instance, acute lymphoblastic leukemia (ALL), acute myeloid leukemia (AML), chronic lymphocytic leukemia (CLL) and chronic myeloid leukemia (CML).

“Administering” is referred to herein as providing the compositions of the present disclosure to a patient. By way of example and not limitation, composition administration, e.g., injection, may be performed by intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration can be, for example, by bolus injection or by gradual perfusion over time. Alternatively, or concurrently, administration may be by the oral route. Additionally, administration may also be by surgical deposition of a bolus or pellet of cells, or positioning of a medical device. In an embodiment, a composition of the present disclosure may comprise engineered cells or host cells expressing nucleic acid sequences described herein, or a vector comprising at least one nucleic acid sequence described herein, in an amount that is effective to treat or prevent proliferative disorders. A pharmaceutical composition may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the terms “treatment,” “treating,” and its grammatical equivalents refer to obtaining a desired pharmacologic and/or physiologic effect. In embodiments, the effect is therapeutic, i.e., the effect partially or completely cures a disease and/or adverse symptom attributable to the disease. To this end, the method described herein comprises administering a “therapeutically effective amount” of the composition comprising the host cells expressing the nucleic acid sequence described herein, or a vector comprising the nucleic acid sequences described herein.

The terms “therapeutically effective amount”, “therapeutic amount”, “immunologically effective amount”, anti-tumor effective amount”, “tumor inhibiting effective amount” or their grammatical equivalents refers to an amount effective, at dosages and for periods of time necessary, to achieve a desired therapeutic result. The therapeutically effective amount may vary according to factors such as the disease state, age, sex, and weight of the individual and the ability of a composition described herein to elicit a desired response in the individual. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject may be “prophylactic,” i.e., the effect completely or partially prevents a disease or symptom thereof.

A “prophylactically effective amount” refers to an amount effective, at dosages and for periods of time necessary, to achieve a desired prophylactic result (e.g., prevention of disease onset).

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and An anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Nucleic Acid Molecules Encoding Antibodies

Another aspect of the present disclosure pertains to isolated nucleic acid sequences that encode the antibody polypeptide, described herein or antigen-binding fragment thereof “Polynucleotide,” or “nucleic acid molecule,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A nucleic acid molecule can comprise modified nucleotides, such as methylated nucleotides and their analogs. A nucleic acid molecule is “isolated” or “rendered substantially pure” when purified away from other cellular components or other contaminants, e.g., other cellular nucleic acids or proteins, by standard techniques, including, but not limited to alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid according to at least some embodiments of the disclosure can be, for example, DNA or RNA and may or may not contain intronic sequences. In a preferred embodiment, the nucleic acid is a cDNA molecule.

In some embodiments, the isolated nucleic acid molecule comprises a nucleic acid sequence encoding a heavy chain variable domain of an antibody. In some embodiments, the nucleic acid sequence encoding a second variable light chain domain comprises a nucleotide sequence of SEQ ID NO: 49. In some embodiments, the nucleic acid sequence encoding a second variable light chain domain comprises a nucleotide sequence of SEQ ID NO: 52. In some embodiments, the nucleic acid sequence encoding a first variable light chain domain comprises a nucleotide sequence of SEQ ID NO: 50. In some embodiments, the nucleic acid sequence encoding a first variable light chain domain comprises a nucleotide sequence of SEQ ID NO: 51.

In some embodiments, the isolated nucleic acid molecule comprises a nucleic acid sequence encoding a heavy chain polypeptide of an antibody. In some embodiments, the nucleic acid sequence encoding a second variable heavy chain domain comprises a nucleotide sequence of SEQ ID NO: 46. In some embodiments, the nucleic acid sequence encoding a first variable heavy chain domain comprises a nucleotide sequence of SEQ ID NO: 47. In some embodiments, the nucleic acid sequence encoding a first variable heavy chain domain comprises a nucleotide sequence of SEQ ID NO: 48.

In some embodiments, the isolated nucleic acid molecule encoding a first polypeptide in a dual variable domain Ig comprises a nucleotide sequence of SEQ ID NO: 53. In some embodiments, the isolated nucleic acid molecule encoding a second polypeptide in a dual variable domain Ig comprises a nucleotide sequence of SEQ ID NO: 55.

In some embodiments, the isolated nucleic acid molecule encoding a first polypeptide in a dual variable domain Ig comprises a nucleotide sequence of SEQ ID NO: 54. In some embodiments, the isolated nucleic acid molecule encoding a second polypeptide in a dual variable domain Ig comprises a nucleotide sequence of SEQ ID NO: 56.

In some embodiments, the isolated nucleic acid encoding a polypeptide of the antibody construct comprises a nucleotide sequence selected from Table 15, and Table 16. In some embodiments, the isolated nucleic acid encoding a IgA heavy chain constant domain is selected from SEQ ID NOs: 61-66. In some embodiments, the isolated nucleic acid encoding a light chain constant domain of the Kappa type comprises a nucleotide sequence of SEQ ID NO: 67 or SEQ ID NO: 68. In some embodiments, the isolated nucleic acid encoding a light chain constant domain of the Lambda type comprises a nucleotide sequence of SEQ ID NO: 69.

Nucleic acid molecules according to at least some embodiments of the present disclosure can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Once DNA fragments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame. The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgA1 or IgA2 constant region. The VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a peptide linker, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

Nucleic acid molecules isolated from the present disclosure can include nucleic acid molecules comprising an open reading frame (ORF), optionally with one or more introns, e.g., but not limited to, at least one specified portion of at least a CDR, as CDR1, CDR2 and/or CDR3 of at least one light chain or at least one heavy chain; nucleic acid molecules comprising the coding sequence of an antibody construct disclosed herein or variable region e.g., variable regions of the light chain and variable regions of the heavy chain; and nucleic acid molecules comprising a nucleotide sequence substantially different from those described above but which, due to the degeneracy of the genetic code, still encode at least antibody or antigen binding fragment thereof or domains or polypeptides of antibody as described herein and/or as it is known in the art. Of course, the genetic code is well known in the art. Therefore, it would be routine for one skilled in the art to generate such degenerate nucleic acid variants encoding specific antibodies of the present disclosure. See for example, Ausubel et al., Supra, and such nucleic acid variants are included in the present invention.

Nucleic acid molecules comprising nucleic acid sequence that encode one or more domains of an antibody are provided herein. In some embodiments, a nucleic acid molecule comprises a nucleic acid sequence that encodes a heavy chain variable domain or a light chain variable domain of an antibody. In some embodiments, a nucleic acid molecule comprises both a nucleic acid sequence that encodes a heavy chain variable domain and a nucleic acid sequence that encodes a light chain variable domain, of an antibody. In some embodiments, a first nucleic acid molecule comprises a first nucleic acid sequence that encodes a heavy chain variable domain and a second nucleic acid molecule comprises a second nucleic acid sequence that encodes a light chain variable domain.

In some embodiments, the heavy variable domain and the light chain variable domain are expressed from one nucleic acid molecule, or from two separate nucleic acid molecules, as two separate polypeptides. In some embodiments, such as when an antibody is an scFv, a single nucleic acid sequence encodes a single polypeptide comprising both a heavy chain variable domain and a light variable domain chain linked together.

In some embodiments, the nucleic acid molecule is one that encodes for any of the amino acid sequences for the antibodies in the Tables 1-2 herein. In some embodiments, the nucleic acid sequence is one that is at least 80% identical to a nucleic acid encoding any of the amino acid sequences in the Tables 12-17 herein, for example, at least 80, 85, 90, 91, 92, 93, 94, 95, 96, 97, 98, or 99% identical. In some embodiments, the nucleic acid is one that hybridizes to any one or more of the nucleic acid sequences provided herein. In some of the embodiments, the hybridization is under moderate conditions. In some embodiments, the hybridization is under highly stringent conditions, such as: at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

Nucleic acid molecules can be constructed using recombinant DNA techniques conventional in the art. In some embodiments, a nucleic acid molecule is placed in an expression vector that is suitable for expression in a selected host cell.

Vectors comprising nucleic acid molecules that encode the antibodies or antigen binding fragment herein are provided. Vectors comprising nucleic acid molecules that encode a heavy chains and/or a light chains are also provided. Such vectors include, but are not limited to, DNA vectors, phage vectors, viral vectors, retroviral vectors, etc. In one embodiment, the nucleic acid coding for the light variable domain and that coding for the heavy chain variable domain are isolated separately by the procedures outlined above. In one embodiment, the isolated nucleic acid encoding the light chain variable domain and that coding for the heavy chain variable domain may be inserted into separate expression plasmids, or together in the same plasmid, so long as each is under suitable promoter and translation control. In some embodiments, the heavy chain variable domain and light chain variable domain are expressed as part of a single polypeptide, such as, for example, when the antibody is an scFv.

In some embodiments, a first vector comprises a nucleic acid molecule that encodes a heavy chain variable domain and a second vector comprises a nucleic acid molecule that encodes a light chain variable domain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used. In some embodiments, a vector is selected that is optimized for expression of polypeptides in CHO or CHO-derived cells, or in NSO cells. Exemplary such vectors are described, for example, in Running Deer et al., Biotechnol. Prog. 20:880-889 (2004).

In one aspect, the present disclosure provides methods for treatment or prevention of cancer comprising administering nucleic acid molecules, wherein the nucleic acid molecules encode for a VH, VL, CDR3 region of VH or CDR 3 region of VL or antigen binding fragment thereof, wherein the nucleic acid molecule comprises a sequence disclosed herein (e.g. Tables 12-17) by way of gene therapy. Gene therapy refers to therapy performed by the administration to a subject of an expressed or expressible nucleic acid. In this embodiment of the invention, the nucleic acids produce their encoded protein that mediates a prophylactic or therapeutic effect. Any of the methods for gene therapy available in the art can be used according to the embodiments herein.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215 Methods. commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY. Delivery of a therapeutic antibody to appropriate cells can be effected via gene therapy ex vivo, in situ, or in vivo by use of any suitable approach known in the art, including by use of physical DNA transfer methods (e.g., liposomes or chemical treatments) or by use of viral vectors (e.g., adenovirus, adeno-associated virus, or a retrovirus).

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

Other in vivo nucleic acid transfer techniques include transfection with viral vectors (such as adenovirus, Herpes simplex I virus, or adeno-associated virus) and lipid-based systems. The nucleic acid and transfection agent are optionally associated with a microparticle. Exemplary transfection agents include calcium phosphate or calcium chloride co-precipitation, DEAE-dextran-mediated transfection, quaternary ammonium amphiphile DOTMA ((dioleoyloxypropyl) trimethylammonium bromide, commercialized as Lipofectin by GIBCO-BRL))(Felgner et al, (1987) Proc. Natl. Acad. Sci. USA 84, 7413-7417; Malone et al. (1989) Proc. Natl Acad. Sci. USA 86 6077-6081); lipophilic glutamate diesters with pendent trimethylammonium heads (Ito et al. (1990) Biochem. Biophys. Acta 1023, 124-132); the metabolizable parent lipids such as the cationic lipid dioctadecylamido glycylspermine (DOGS, Transfectam, Promega) and dipalmitoylphosphatidyl ethanolamylspermine (DPPES)(J. P. Behr (1986) Tetrahedron Lett. 27, 5861-5864; J. P. Behr et al. (1989) Proc. Natl. Acad. Sci. USA 86, 6982-6986); metabolizable quaternary ammonium salts (DOTB, N-(1-[2,3-dioleoyloxy]propyl)-N,N,N-trimethylammonium methylsulfate (DOTAP)(Boehringer Mannheim), polyethyleneimine (PEI), dioleoyl esters, ChoTB, ChoSC, DOSC)(Leventis et al. (1990) Biochim. Inter. 22, 235-241); 3beta[N—(N′,N′-dimethylaminoethane)-carbamoyl]cholesterol (DC-Chol), dioleoylphosphatidyl ethanolamine (DOPE)/3beta[N—(N′,N′ dimethylaminoethane)-carbamoyl] cholesterolDC-Chol in one to one mixtures (Gao et al., (1991) Biochim. Biophys. Acta 1065, 8-14), spermine, spermidine, lipopolyamines (Behr et al., Bioconjugate Chem, 1994, 5: 382-389), lipophilic polylysines (LPLL) (Zhou et al., (1991) Biochim. Biophys. Acta 939, 8-18), [[(1, 1,3,3 tetramethylbutyl)cresoxy]ethoxy]ethyl]dimethylbnzylammonium hydroxide (DEBDA hydroxide) with excess phosphatidylcholine/cholesterol (Ballas et al., (1988) Biochim. Biophys. Acta 939, 8-18), cetyltrimethylammonium bromide (CTAB)/DOPE mixtures (Pinnaduwage et al, (1989) Biochim. Biophys. Acta 985, 33-37), lipophilic diester of glutamic acid (TMAG) with DOPE, CTAB, DEBDA, didodecylammonium bromide (DDAB), and stearylamine in admixture with phosphatidylethanolamine (Rose et al., (1991) Biotechnique 10, 520-525), DDAB/DOPE (TransfectACE, GIBCO BRL), and oligogalactose bearing lipids. Exemplary transfection enhancer agents that increase the efficiency of transfer include, for example, DEAE-dextran, polybrene, lysosome-disruptive peptide (Ohmori N I et al, Biochem Biophys Res Commun Jun. 27, 1997; 235(3):726-9), chondroitan-based proteoglycans, sulfated proteoglycans, polyethylenimine, polylysine (Pollard H et al. J Biol Chem, 1998 273 (13):7507-11), integrin-binding peptide CYGGRGDTP, linear dextran nonasaccharide, glycerol, cholesteryl groups tethered at the 3′-terminal internucleoside link of an oligonucleotide (Letsinger, R. L. 1989 Proc Natl Acad Sci USA 86: (17):6553-6), lysophosphatide, lysophosphatidylcholine, lysophosphatidylethanolamine, and 1-oleoyl lysophosphatidylcholine.

In some situations, it may be desirable to deliver the nucleic acid with an agent that directs the nucleic acid containing vector to target cells. Such “targeting” molecules include antibodies specific for a cell-surface membrane protein on the target cell, or a ligand for a receptor on the target cell. Where liposomes are employed, proteins which bind to a cell-surface membrane protein associated with endocytosis may be used for targeting and/or to facilitate uptake. Examples of such proteins include capsid proteins and fragments thereof tropic for a particular cell type, antibodies for proteins which undergo internalization in cycling, and proteins that target intracellular localization and enhance intracellular half-life. In other embodiments, receptor-mediated endocytosis can be used. Such methods are described, for example, in Wu et al., 1987 or Wagner et al., 1990. For review of the currently known gene marking and gene therapy protocols, see Anderson 1992. See also WO 93/25673 and the references cited therein.

### EXAMPLES

These examples are provided for illustrative purposes only and not to limit the scope of the claims provided herein.

### Example 1. IgA Based Antibody Construct Induce Phagocytosis of Tumor Cells

A CD20-CD47 IgA bispecific antibody construct enables phagocytosis of tumor cells as a result of blocking the CD47-SIRPα interaction and engaging FcRs on macrophages. CD20+CD47+ CFSE-labeled cell lines are co-incubated with unlabeled human macrophages in the presence of different antibodies, and phagocytosis of target cells is assessed by flow cytometry. Antibodies directed against CD47 or CD20 induce significant phagocytosis relative to the baseline level observed with isotype control antibody. IgA bispecific antibodies recapitulate the synergy of the anti-CD47 and rituximab combination treatment and increase phagocytosis relative to treatment with anti-CD47 alone in the cell lines that are tested.

### Example 2 Bispecific Antibody Constructs

### Dual Variable Domain IgA Immunoglobulin (DVD-Ig Bispecific IgA)

Bispecific antibody constructs capable of binding CD20 and CD47 that were dual variable domain IgA immunoglobulin (DVD-Ig bispecific IgA) were generated. In these bispecific antibody constructs, additional variable light chain domain (VL) and variable heavy chain domain (VH) domain were linked on top of the existing IgA VH-VL domains by means of a short linker (SEQ ID NO: 44; SGGGGS). The additional VH domain is linked to the pre-existing VH, likewise the additional VL is linked to the pre-existing VL. In addition to these bispecific constructs, two single-linked dual variable domain antibodies, employing a scFv format were also prepared (DVD-IgA scFv). The scFv element by itself links the VL to the VH with a long linker (SEQ ID NO: 45; GGGGSGGGGSGGGGS), whereas the total scFv molecule is linked to either a VL or a VH by the short linker (SEQ ID NO:44; SGGGGS).

The antigen CD20 binding variable domains were derived from Obinituzumab (a CD20 binding antibody). The CD47 binding variable domains were derived from the 5A3M5 or 2.3D11 clones (both CD47 binding antibodies). An overview of all combinations can be found in Table 1 below

The positioning of the additional CD20 and CD47 variable domains were used in both orientations, using the following combinations:

### Dual-Linked Bispecific IgA:

In one bispecific antibody construct (bispecific antibody #2), additional VH/VL domain positioned in the outer domain contained the CD20 binding moiety (i.e., CD20 binding variable domains), and the pre-existing VH/VL domain contained the aCD47 2.3D11 binding moiety (i.e., CD47 binding variable domains).

In another bispecific antibody construct (bispecific antibody #1), additional VH/VL domain positioned in the outer domain contains the aCD47 5A3M5 binding moiety (i.e., CD47 binding variable domains), and the pre-existing VH/VL domain contains the aCD20 binding moiety.

scFv-LC Single Linked Bispecific IgA

Bispecific antibody #6 had the scFv positioned in the outer domain. The scFV contained the CD20 binding moiety, and the pre-existing VH/VL domain in the bispecific antibody construct contained the aCD47 2.3D11 binding moiety. The scFv was linked to the variable light chain, specifically variable light chain domain of the aCD47 2.3D11 binding moiety.

Bispecific antibody #5 had the scFv positioned in the outer domain. The scFv contained the aCD47 5A3M5 binding moiety, and the pre-existing VH/VL domain contained the CD20 binding moiety. The scFv was linked to the variable light chain, specifically variable light chain domain of the CD20 binding moiety.

scFv-HC Single Linked Bispecific IgA

Bispecific antibody #4 had the scFv positioned in the outer domain. The scFV contained the CD20 binding moiety, and the pre-existing VH/VL domain contained the aCD47 2.3D11 binding moiety. The scFv was linked to the variable heavy chain, specifically variable heavy chain domain of the CD 47 binding moiety (aCD47 2.3D11 binding moiety).

Bispecific antibody #3 had the scFv positioned in the outer domain. The scFv contained the CD47 binding moiety (aCD47 5A3M5 binding moiety), and the pre-existing VH/VL domain contained the CD20 binding moiety. The scFv was linked to the variable heavy chain, specifically variable heavy chain domain of the CD20 binding moiety.

TABLE 1
lists the specificities and orientations of DVD-Ig bispecific IgA.

| Ab format | Bispecific Ab | Target outer domain | Target inner domain |
|---|---|---|---|
| DVD-IgA | #1 (5A3M5-Obi) | 5A3M5 | Obinituzumab |
| | | (CD47 binding domain) | (Exemplary antigen |
| | | | CD20 binding domain) |
| | #2 (Obi-2.3D11) | Obinituzumab | 2.3D11 |
| | | (Exemplary antigen | (CD47 binding domain) |
| | | CD20 binding domain) | |
| DVD-IgA | #3 (5A3M5-scFv: Obi_HC) | 5A3M5 | Obinituzumab |
| scFv_HC | | (CD47 binding domain) | (Exemplary antigen |
| | | | CD20 binding domain) |
| | #4 (Obi-scFv: 2.3D11_HC) | Obinituzumab | 2.3D11 |
| | | (Exemplary antigen | (CD47 binding domain) |
| | | CD20 binding domain) | |
| DVD-IgA | #5 (5A3M5-scFv: Obi_LC) | 5A3M5 | Obinituzumab |
| scFv_LC | | (CD47 binding domain) | (Exemplary antigen |
| | | | CD20 binding domain) |
| | #6 (Obi-scFv: 2.3D11_LC) | Obinituzumab | 2.3D11 |
| | | (Exemplary antigen | (CD47 binding domain) |
| | | CD20 binding domain) | |

### Kappa-Lambda Bispecific Antibodies

Kappa-lambda bispecific antibodies were also generated. In these antibodies, heavy chain which does not have any affinity against anything was used in combination with both kappa and lambda light chains, each containing a separate affinity in their VL domains. Hence, the dual specificity was directed by the light chains only, which in this case is CD19 by the C2 clone and CD47 by the 5A3M5 clone. The VH region of the common heavy chain was tailored to an IgA molecule. The kappa and lambda light chains were unmodified. A set of control light chains not containing any affinity nor specificity were used to determine single arm affinity, so-called Dummy light chains. The table 2 below depicts exemplary combinations of kappa-lambda antibodies.

TABLE 2
lists specificities and combinations of kappa-lambda IgA bispecific antibodies

| Ab format | Kappa-lambda Ab | Kappa specificity | Lambda specificity |
|---|---|---|---|
| Kappa-lambda IgA | #7 (5A3M5-C2) | 5A3M5 | C2 |
| | | (CD47 binding domain) | (Exemplary antigen |
| | | | CD19 binding domain) |
| | #8 (5A3M5-dummy) | 5A3M5 | Dummy |
| | | (CD47 binding domain) | |
| | #9 (Dummy-C2) | Dummy | C2 |
| | | | (Exemplary antigen |
| | | | CD19 binding domain) |
| | #10 (dummy-dummy) | Dummy | Dummy |

### Example 3. Generation of Bispecific Antibody Constructs

### Cloning of IgA Bispecific Antibody Constructs

### DVD-IgA3.0

The fragment consisting of 5A3M3 VH and Obinituzumab VH containing a SGGGGS linker (SEQ ID NO: 44) (bispecific antibody #1) joining the two VH's to each other was codon optimized for use in Cricetulus griseus cells, ordered as synthetic DNA (gBlock) at IDT, and cloned into a pEE14.4 vector containing the constant region of IgA3.0 pre-digested with HindIII and NotI using HiFi assembly (NEB) according to manufacturer's protocol.

The fragment consisting of Obinituzumab VH and 2.3D11 VH containing a SGGGGS linker (SEQ ID NO:44) (bispecific antibody #2) joining the two VH's to each other was codon optimized for use in Cricetulus griseus cells, ordered as synthetic DNA (gBlock) at IDT, and cloned into a pEE14.4 vector containing the constant region of IgA3.0 pre-digested with HindIII and NotI using HiFi assembly (NEB) according to manufacturer's protocol.

The fragment consisting of 5A3M3 VL and Obinituzumab VL containing a SGGGGS linker (SEQ ID NO: 44) (bispecific antibody #1) joining the two VL's to each other was codon optimized for use in Cricetulus griseus cells, ordered as synthetic DNA (gBlock) at IDT, and cloned into a pEE14.4 vector containing the constant region of IgA3.0 pre-digested with HindIII and NotI using HiFi assembly (NEB) according to manufacturer's protocol.

The fragment consisting of Obinituzumab VL and 2.3D11 VL containing a SGGGGS linker (SEQ ID NO: 44) (bispecific antibody #2) joining the two VL's to each other was codon optimized for use in Cricetulus griseus cells, ordered as synthetic DNA (gBlock) at IDT, and cloned into a pEE14.4 vector containing the constant region of IgA3.0 pre-digested with HindIII and NotI using HiFi assembly (NEB) according to manufacturer's protocol.

DVD-IgA scFv_HC

The scFv fragment consisting of 5A3M5 VH linked via GGGGSGGGGSGGGGS (SEQ ID NO: 45) to 5A3M5 VL and subsequently linked to Obinituzumab VH by a SGGGGS (SEQ ID NO: 44) linker (bispecific antibody #3), joining the scFv to the VH has been codon optimized for use in Cricetulus griseus cells, ordered as synthetic DNA (gBlock) at IDT, and cloned into a pEE14.4 vector containing the constant region of IgA3.0 pre-digested with HindIII and NotI using HiFi assembly (NEB) according to manufacturer's protocol.

The scFv fragment consisting of Obinituzumab VH linked via GGGGSGGGGSGGGGS (SEQ ID NO: 45) to Obinituzumab VL and subsequently linked to 2.3D11 VH by a SGGGGS linker (SEQ ID NO: 44)(bispecific antibody #4), joining the scFv to the VH was codon optimized for use in Cricetulus griseus cells, ordered as synthetic DNA (gBlock) at IDT, and cloned into a pEE14.4 vector containing the constant region of IgA3.0 pre-digested with HindIII and NotI using HiFi assembly (NEB) according to manufacturer's protocol.

The Obinituzumab VL or VH has been cloned in a pBluescript II vector, from which the VH or VL has been subcloned via HindIII-NotI into the pEE14.4 vector containing the constant region of IgA3.0 or kappa light chain.

The 2.3D11 VL or VH has been cloned in a pBluescript II vector, from which the VH or VL has been subcloned via HindIII-NotI into the pEE14.4 vector containing the constant region of IgA3.0 or kappa light chain.

DVD-IgA scFv_LC

The scFv fragment consisting of 5A3M5 VH linked via GGGGSGGGGSGGGGS (SEQ ID NO: 45) to 5A3M5 VL and subsequently linked to Obinituzumab VL by a SGGGGS linker (SEQ ID NO: 44) (bispecific antibody #5), joining the scFv to the VH has been codon optimized for use in Cricetulus griseus cells, ordered as synthetic DNA (gBlock) at IDT, and cloned into a pEE14.4 vector containing the constant region of IgA3.0 pre-digested with HindIII and NotI using HiFi assembly (NEB) according to manufacturer's protocol.

The scFv fragment consisting of Obinituzumab VH linked via GGGGSGGGGSGGGGS (SEQ ID NO: 45) to Obinituzumab VL and subsequently linked to 2.3D11 VL by a SGGGGS linker (SEQ ID NO: 44) (bispecific antibody #6), joining the scFv to the VH was codon optimized for use in Cricetulus griseus cells, ordered as synthetic DNA (gBlock) at IDT, and cloned into a pEE14.4 vector containing the constant region of IgA3.0 pre-digested with HindIII and NotI using HiFi assembly (NEB) according to manufacturer's protocol.

### Kappa-Lambda Antibodies

A full IgA3.0 common heavy chain was codon optimized for use in Cricetulus griseus cells, ordered as synthetic DNA and assembled into a pcDNA3.4 vector using HiFi assembly (NEB) according to manufacturer's protocol.

A full 5A3M5 kappa light chain was codon optimized for use in Cricetulus griseus cells, ordered as synthetic DNA and assembled into a pcDNA3.4 vector using HiFi assembly (NEB) according to manufacturer's protocol.

A full C2 lambda light chain was codon optimized for use in Cricetulus griseus cells, ordered as synthetic DNA and assembled into a pcDNA3.4 vector using HiFi assembly (NEB) according to manufacturer's protocol.

### Transfection of HEK293F Cells

HEK293 Freestyle cells were transfected with the following modifications: A ratio of DNA:293fectin of 1:1.33 was used. Several antibody chain ratios of HC:LC DNA. i.e., DNA encoding the heavy chain and DNA encoding the light chain were tested in combination with pAdvantage (Promega) in a total concentration of 1 ug/mL in a volume of 2 mL. The amount of pAdvantage DNA was always equal to heavy chain DNA.

The production of the supernatant used in the FACS, ADCC, RBC and platelet profiling experiments were produced in 4 mL in the following ratios: #1 (1:1:1), #2(1:2:1), #3 (1:0,5:1), #4 (1:0,5:1), #5 (1:2:1), #6 (1:2:1).

Cell producing antibodies were harvested, centrifuged for 5 minutes at 350 g and the resulting supernatant was collected. The supernatant was clarified of any cell debris by passing through a 0.22 um filter.

### ELISA

Concentrations of bispecific antibodies were then measured in the clarified supernatant obtained above by ELISA as follows;

### Day 1: Plate Coating

Plates were coated with antibody in PBS o/n @ 4′C, use 100 μL/well.

### Day 2: ELISA

Plate was washed 3× with 150 μL/well of Wash buffer, Plate was gently flicked after each wash. To block plate 150 μL/well Block buffer was added to each well, and incubated for 1 hr at room temperature. The plate was gently flicked and standards were added. Samples were diluted in Blocking buffer according to following conditions and incubated for 2 hr at RT; standard dilutions; 100 μL/well and diluted samples; 100 μL/well.

Plate was flicked and then washed 3× with 150 μL/well of Wash buffer. Detection antibody was added in Blocking buffer; 100 μL/well, and incubated 1 hr at RT. Plate was flicked and washed 3× with 150 μL/well of Wash buffer, plate was flicked after each wash. ABTS substrate was added (1 tablet of 50 mg in 50 mL ABTS buffer, store in dark); 100 μL/well for ±15 min. Plate was measured on Bio-Rad spectrophotometer at OD 415 nm.

Buffers used were; Wash buffer (PBS, 0.05% Tween-20) and Blocking buffer (PBS, 0.05% Tween-20, 1% BSA). PBS used; Sigma; D88537. Coating antibody was Goat-anti-human kappa; Southern Biotech; 2060-01 used 1:2000. Coating antibody used was Goat-anti-human lambda; Southern Biotech; 2070-01 used 1:2000. Standard curve human IgA; BETHYL; p80-102. Detection antibody used was Goat-anti-human IgA HRP; Southern Biotech; 2050-05 used 1:2000. Detection antibody used was: Goat-anti-human lambda HRP; Southern Biotech; 2070-05 used 1:5000. MaxiSORP plates were used for assay; NUNC; 439454 and BSA; Roche; 10735094001.

For the DVD-IgA, DVD-IgA-scFv and kappa-kappa antibody molecules the Goat-anti-human kappa coating antibody was used in combination with Goat-anti-human IgA-HRP detection antibody. For the lambda-lambda antibodies the Goat-anti-human lambda coating antibody was used in combination with Goat-anti-human IgA-HRP detection antibody. For the kappa-lambda antibodies the Goat-anti-human kappa coating antibody was used in combination with Goat-anti-human lambda-HRP detection antibody.

### Purification of Bispecific Antibodies by HiTrap KappaSelect

Supernatant was diluted in PBS 1:1 prior to loading onto the 5 mL HiTrap Kappa Select column (17-5458-12; GE Healthcare). After loading of entire sample column was rinsed in 5 column volumes (CV) of PBS, followed by elution buffer (0.1M Glycine pH2.5). Fractions of 1 mL were collected in tubes pre-filled with neutralization buffer (75 uL 1M Tris pH 8.8).

### SDS-PAGE

A total of 15 uL per HiTrap Kappa Select eluate was added to 5 uL 4x loading buffer (Bio-Rad) without reducing agents and ran on a 4-20% Precast gradient gel MiniProtean TGX (Bio-Rad) for ±1 hour. Gel was stained in InstantBlue (1SB1L; Expedeon) on a rocker until bands were visible, washed three times with water for 5 minutes on a rocker and scanned.

### Flow Cytometry Bispecific Antibody Binding Assay

SKBR3 WT and SKBR3-CD20 cells were cultured in RPMI-1640 medium (Gibco) using standard culture conditions. An amount of 100.000 cells were stained with Obi-IgA3.0 (1 ug/mL), or 2.3D11-IgA2 (1 ug/mL), 50 uL bispecific antibody supernatant for 45 minutes at 4′C, followed by a wash in FACS buffer (1% BSA in PBS). A secondary antibody was used to detect the IgA molecules, using a Goat anti-hIgA RPE F(ab′)2 (Southern Biotech 2052-09; used 1:150) for 45 minutes at 4′C, followed by a wash in FACS buffer. Part of the cells were pre-blocked with mIgG1 anti-CD47 PerCP-Cy5.5 (Biolegend; clone CC26C; use 1:20) for 45 minutes at 4′C, followed by a wash. After the pre-block the IgA antibodies were applied as described above. Samples were analyzed on a BD Canto II, and data has been analyzed using FlowJo (BD).

### Antibody-Dependent Cellular Cytotoxicity (ADCC)

A total of 1 million target cells were labeled with 51Cr for at least 3 hours, followed by a wash in culture medium. A subset (500.000) of target cells were pre-blocked by the mIgG1 anti-CD47 PerCp-Cy5.5 antibody by incubating for 1 hour at 4′C in 200 uL 20x diluted, followed by a culture medium wash.

Effector cells were isolated form healthy donors using standard Ficoll-paque (GE Healthcare) and Histopaque-1119 (Sigma Aldrich). Histopaque was applied on top of the Ficoll layer prior to the 1:1 in PBS diluted blood mixture application. Tubes were spun down for 25 minutes at 450 g at RT. PMN's were isolated from the Histopaque layer, and washed in culture medium.

A total of 200.000 PMN's were used to be co-cultured for 4 hours with 5000 target cells (1:40 ratio) in the presence of various concentrations of antibodies (Obi-IgA3.0; 2.3D11-IgA3.0), or raw supernatants of the bispecific antibodies. As a minimal release target and effector cells only were taken, whereas for maximal release target and effector cells were cocultured in 5% Triton.

### Example 4. Generation and Characterization of Bispecific Antibodies

All 10 forms of the bispecific antibodies were produced in HEK293F cells using standard conditions and clarified supernatant was harvested, passed through a 0.22 um filter, and stored at 4′C until use. Supernatants were assessed by ELISA to determine successful production and antibody concentrations. Productions of all bispecific molecules were successful and resulted in sufficient concentrations to be used in downstream assays.

For the DVD-Ig like bispecific antibodies (#1-6) raw supernatants were applied directly to the following cells: 1) SKBR3 cells naturally expressing CD47 but not CD20 (CD20−/CD47+); 2) SKBR3 cell line transduced with CD20, acting as a double-positive (CD20+/CD47+) cell line.

A flow cytometric analysis was performed to detect the IgA-bispecific antibodies on the cell surface.

In order to determine what the efficiency is regarding CD47 opsonization by bispecific antibodies and whether the additional binding capacity to CD47 can enhance the CD20 binding of the bispecific antibodies, an mIgG1 blocking antibody against CD47 (labeled with PerCP-Cy5.5) was used to either post- or pre-block the CD47 molecule respectively. This was a different clone than is used in the IgA variants. Also a control was taken along that determines the opsonization of CD20 by the bispecifics, for this IgG1 Obinituzumab was used.

For the pre-block (determining enhanced CD20 binding by bispecific antibodies) the following method was used: 1) the CD47 blocking antibody was applied onto the cells, the non-bound antibody fraction was washed away; 2) cells were incubated with bispecific antibodies (supernatant), the non-bound bispecific antibody fraction was washed away; and 3) cells were incubated with anti-IgA-PE, the non-bound antibody fraction was washed away. Cells were fixed and measured by FACS.

For the post-block (determining the CD47 opsonization by bispecific antibodies) the following method was used: 1) cells were incubated with bispecific antibodies (supernatant), the non-bound bispecific antibody fraction was washed away; and 2) cells were incubated with anti-IgA-PE, at the same time the CD47 blocking antibody was applied onto the cells, the non-bound antibody fraction was washed away. Cells were fixed and measured by FACS. Controls that have been taken along are unstained cells, secondary antibody only, and bivalent monospecific antibodies.

In the SKBR3 cells a strong binding of CD47 of the bivalent monospecific antibody 2.3D11 was observed as expected. A crossblocking experiment using the mIgG1 aCD47 antibody showed CD47 could be blocked successfully, no binding by bispecific antibody could be observed. Although, binding of bispecific #4 and #6 was seen, yet they do not fully opsonize CD47 on the cells as observed in the post blocking experiment.

In the SKBR3-CD20 all bispecific antibodies bind CD20 with high affinity, but do not fully opsonize the cells. The CD47 molecule has also not been fully opsonized as determined by post-blocking with an anti-CD47 mIgG1. However, when the CD47 molecule was pre-block and then stained with the bispecific antibodies a decrease in binding is observed for antibody #2, #3, and #5. These antibodies do not show binding of CD47 as observed on CD20-negative cells (SKBR3-WT). This shows that by binding the CD47 molecule, the bispecific antibodies this are better able to bind CD20, showing enhanced binding. The observations above indicate that IgA antibodies can be tailored into bispecific antibodies, binding two targets simultaneously, where in these examples CD47 binding enhances CD20 binding.

### Erythrocyte Staining

A Histopaque/ficoll isolation was performed to separate the erythrocytes from the PMN/PBMC. Plasma, PBMC, ficoll, histopaque and PMN have been discarded. The remaining pellet of erythrocytes were washed in PBS, spun for 5 minutes at 2400 rpm and pellet was resuspended in 5 mL PBS. An amount of 10 uL per sample was used for staining with mouse IgG2b anti-human CD235a-PE (BD; clone GA-R2; dilution 1:40) combined with mouse IgG1 anti human CD47 PerCP-Cy5.5 (Biolegend; clone CC2C6; dilution 1:20), or 40 uL supernatant of the bispecific IgA antibodies 1-6. Dilutions are done in FACS buffer (1% BSA in PBS). Incubated for 30 minutes at RT in the dark, excess unbound antibody was washed away with FACS buffer. 40 uL was added in FACS buffer diluted detection antibody (goat F(ab)′2 anti-human IgA-FITC (Southern Biotech; dilution 1:100), incubated for 30 minutes at RT in the dark, excess unbound antibody was washed away with FACS buffer. Samples were fixed by adding 1% PFA to pelleted cells. Measure on Canto II (BD), gating on CD235-positive events.

### Platelet Staining

10 uL whole blood was used from a Heparin/EDTA tube per sample for staining with mouse IgG1 anti-human CD61-FITC (Dako; clone Y2-51; dilution 1:20) combined with mouse IgG1 anti human CD47 PerCP-Cy5.5 (Biolegend; clone CC2C6; dilution 1:20), or 40 uL supernatant of the bispecific IgA antibodies 1-6. Dilutions were done in FACS buffer (1% BSA in PBS). Incubated for 30 minutes at RT in the dark, then 0.5 uL detection antibody (goat F(ab)′2 anti-human IgA-RPE (Southern Biotech; dilution 1:50) was added and incubated for 30 minutes at RT in the dark. Samples were fixed by adding 1% PFA to pelleted cells. Measurements were done on Canto II (BD), gating on CD61-positive events.

TABLE 3
lists amino acid sequences of complementarity
determining regions of the variable heavy chains

| | SEQ | | SEQ | | SEQ | |
|---|---|---|---|---|---|---|
| | ID | | ID | | ID | |
| Name | NO: | CDR-H1 | NO: | CDR-H2 | NO: | CDR-H3 |
| Obinituzumab_VH | 1 | GYAFSY | 2 | RIFPGDG | 3 | NVFDGYWL |
| | | S | | DTDYNG | | VY |
| 2.3D11_VH | 4 | GVSIRS | 5 | EIYHSGS | 6 | DGGIAVTD |
| | | IN | | TNYNPSL | | YYYYGLDV |
| | | | | KS | | |
| Common_VH | 7 | GFTFSS | 8 | AISGSGG | 9 | SYGAFDY |
| | | YAMS | | STYYADS | | |
| | | | | VKG | | |

TABLE 4
lists amino acid sequences of complementarity
determining regions of the variable light chain

| | SEQ | | SEQ | | SEQ | |
|---|---|---|---|---|---|---|
| | ID | | ID | | ID | |
| Name | NO: | CDR-L1 | NO: | CDR-L2 | NO: | CDR-L3 |
| Obinituzumab_VL | 10 | RSSKSLLHS | 11 | QMSNLVS | 12 | AQNLELPY |
| | | NGITYLY | | | | T |
| 2.3D11_VL | 13 | RASESVSSN | 14 | GAFNRAT | 15 | QQRSDWFT |
| | | LA | | | | |
| 5A3M5_VL | 16 | QASQDINKY | 17 | GASRLET | 18 | QQKHPRYP |
| | | LN | | | | RT |
| C2_VL | 19 | TRSSGSIED | 20 | YDNERPS | 21 | QTYDQSLY |
| | | KYVQ | | | | GWV |

TABLE 5
lists amino acid sequences of variable
heavy chain (VH).

| SEQ | | |
|---|---|---|
| ID | | |
| NO: | Name | VH |
| 22 | Obinituzumab_ | QVQLVQSGAEVKKPGSSVKVSCKASGYAFSYSW |
| | VH | INWVRQAPGQGLEWMGRIFPGDGDTDYNGKFKG |
| | | RVTITADKSTSTAYMELSSLRSEDTAVYYCARN |
| | | VFDGYWLVYVVGQGTLVTVSS |
| 23 | 2. 3D11_VH | QVQLQESGPGLVKPSGTLSLTCAVSGVSIRSIN |
| | | WWNWVRQPPGKGLEWIGEIYHSGSTNYNPSLKS |
| | | RVTISVDKSKNQFSLKLNSVTAADTAVYYCARD |
| | | GGIAVTDYYYYGLDVWGQGTTVTVSS |
| 24 | Common_VH | EVQLLESGGGLVQPGGSLRLSCAASGFTFSSYA |
| | | MSWVRQAPGKGLEWVSAISGSGGSTYYADSVKG |
| | | RFTISRDNSKNTLYLQMNSLRAEDTAVYYCAKS |
| | | YGAFDYVVGQGTLVTVSS |

Sequences in bold are amino acid sequences of CDR-H1, CDR-H2 and CDR-H3 in that order.

TABLE 6
lists amino acid sequences of variable
light chain (VL).

| SEQ | | |
|---|---|---|
| ID | | |
| NO: | Name | VL |
| 25 | Obinituzumab_ | DIVMTQTPLSLPVTPGEPASISCRSSKSLLHSN |
| | VL | GITYLYWYLQKPGQSPQLLIYQMSNLVSGVPDR |
| | | FSGSGSGTDFTLKISRVEAEDVGVYYCAQNLEL |
| | | PYTFGGGTKVEIK |
| 26 | 2.3D11_VL | EIVLTQSPATLSLSPGERATLSCRASESVSSNL |
| | | AWYQQKPGQAPRLLIYGAFNRATGIPARFSGSG |
| | | SGTDFTLTISSLEPEDFAVYYCQQRSDWFTFGG |
| | | GTKVEIK |
| 27 | 5A3M5_VL | DIQMTQSPSSLSASVGDRVTITCQASQDINKYL |
| | | NVVYQQKPGKAPKLLIYGASRLETGVPSRFSGS |
| | | GSGTDFTFTISLQPEDIATYYCQQKHPRYPRTF |
| | | GQGTKVEIK |
| 28 | C2_VL | NFMLTQPHSVSESPGKTVTISCTRSSGSIEDKY |
| | | VQWYQQRPGSSPTIVIYYDNERPSGVPDRFSGS |
| | | IDSSSNSASLTISGLKTEDEADYYCQTYDQSLY |
| | | GWVFGGGTKLTVL |

Sequences in bold are amino acid sequences of CDR-LL CDR-L2 and CDR-L3 in that order.

TABLE 7
lists amino acid sequences of DVD-IgA bispecific
variable heavy chain and variable light chain.

| SEQ | | |
|---|---|---|
| ID | | |
| NO: | Name | Sequence |
| 29 | Obi_VH- | QVQLVQSGAEVKKPGSSVKVSCKASGYAFSYSWI |
| | linker- | NWVRQAPGQGLEWMGRIFPGDGDTDYNGKFKGRV |
| | 2.3D11_VH | TITADKSTSTAYMELSSLRSEDTAVYYCARNVFD |
| | | GYWLVYWGQGTLVTVSSSGGGGSQVQLQESGPGL |
| | | VKPSGTLSLTCAVSGVSIRSINWWNWVRQPPGKG |
| | | LEWIGEIYHSGSTNYNPSLKSRVTISVDKSKNQF |
| | | SLKLNSVTAADTAVYYCARDGGIAVTDYYYYGLD |
| | | VWGQGTTVTVSS |
| 30 | 5A3M5_VH- | EVQLLESGGGLVQPGGSLRLSCAASGFTFSSYAM |
| | linker- | SWVRQAPGKGLEWVSAISGSGGSTYYADSVKGRF |
| | Obi_VH | TISRDNSKNTLYLQMNSLRAEDTAVYYCAKSYGA |
| | | FDYWGQGTLVTVSSSGGGGSQVQLVQSGAEVKKP |
| | | GSSVKVSCKASGYAFSYSWINWVRQAPGQGLEWM |
| | | GRIFPGDGDTDYNGKFKGRVTITADKSTSTAYME |
| | | LSSLRSEDTAVYYCARNVFDGYWLVYWGQGTLV |
| | | TVSS |
| 31 | Obi_VL- | DIVMTQTPLSLPVTPGEPASISCRSSKSLLHSNG |
| | linker- | ITYLYWYLQKPGQSPQLLIYQMSNLVSGVPDRFS |
| | 2.3D11_VL | GSGSGTDFTLKISRVEAEDVGVYYCAQNLELPYT |
| | | FGGGTKVEIK |
| | | SGGGGSEIVLTQSPATLSLSPGERATLSCRASES |
| | | VSSNLAWYQQKPGQAPRLLIYGAFNRATGIPARF |
| | | SGSGSGTDFTLTISSLEPEDFAVYYCQQRSDWFT |
| | | FGGGTKVEIK |
| 32 | 5A3M5_VL- | DIQMTQSPSSLSASVGDRVTITCQASQDINKYLN |
| | linker- | WYQQKPGKAPKLLIYGASRLETGVPSRFSGSGSG |
| | Obi_VL | TDFTFTISSLQPEDIATYYCQQKHPRYPRTFGQG |
| | | TKVEIKSGGGGSDIVMTQTPLSLPVTPGEPASIS |
| | | CRSSKSLLHSNGITYLYWYLQKPGQSPQLLIYQM |
| | | SNLVSGVPDRFSGSGSGTDFTLKISRVEAEDVGV |
| | | YYCAQNLELPYTFGGGTKVEIK |

CDR regions are in bold and linker is italicized.

TABLE 8
lists amino acid sequences of polypeptide
containing scFv linked to variable heavy
chain domain (DVD-IgA scFv_HC).

| SEQ | | |
|---|---|---|
| ID | | |
| NO: | Name | Sequence |
| 33 | Obinituzumab_ | QVQLVQSGAEVKKPGSSVKVSCKASGYAFSYSWINWV |
| | scFv-2.3D11_HC | RQAPGQGLEWMGRIFPGDGDTDYNGKFKGRVTITADK |
| | | STSTAYMELSSLRSEDTAVYYCARNVFDGYWLVYWGQ |
| | | GTLVTVSSGGGGSGGGGSGGGGSDIVMTQTPLSLPVTPG |
| | | EPASISCRSSKSLLHSNGITYLYWYLQKPGQSPQLLIYQ |
| | | MSNLVSGVPDRFSGSGSGTDFTLKISRVEAEDVGVYYC |
| | | AQNLELPYTFGGGTKVEIKSGGGGSQVQLQESGPGLVK |
| | | PSGTLSLTCAVSGVSIRSINWWNWVRQPPGKGLEWIGEI |
| | | YHSGSTNYNPSLKSRVTISVDKSKNQFSLKLNSVTAADT |
| | | AVYYCARDGGIAVTDYYYYGLDVWGQGTTVTVSS |
| 34 | 5A3M5_scFv- | EVQLLESGGGLVQPGGSLRLSCAASGFTFSSYAMSWVR |
| | Obi_HC | QAPGKGLEWVSAISGSGGSTYYADSVKGRFTISRDNSK |
| | | NTLYLQMNSLRAEDTAVYYCAKSYGAFDYWGQGTLVT |
| | | VSSGGGGSGGGGSGGGGSDIQMTQSPSSLSASVGDRVTI |
| | | TCQASQDINKYLNWYQQKPGKAPKLLIYGASRLETGV |
| | | PSRFSGSGSGTDFTFTISSLQPEDIATYYCQQKHPRYPRT |
| | | FGQGTKVEIKSGGGGSQVQLVQSGAEVKKPGSSVKVSC |
| | | KASGYAFSYSWINWVRQAPGQGLEWMGRIFPGDGDT |
| | | DYNGKFKGRVTITADKSTSTAYMELSSLRSEDTAVYYC |
| | | ARNVFDGYWLVYWGQGTLVTVSS |

CDR regions are in bold and linker is italicized.

TABLE 9
lists amino acid sequences of polypeptide containing scFv
linked to variable light chain domain (DVD-IgA scFv_LC).

| SEQ | | |
|---|---|---|
| ID | | |
| NO: | Name | Sequence |
| 35 | Obinituzumab_ | QVQLVQSGAEVKKPGSSVKVSCKASGYAFSYSWINWVRQA |
| | scFv- | pGQGLEWMGRIFPGDGDTDYNGKFKGRVTITADKSTSTAY |
| | 2.3D11_LC | MELSSLRSEDTAVYYCARNVFDGYWLVYWGQGTLVTVSSG |
| | | GGGSGGGGSGGGGSDIVMTQTPLSLPVTPGEPASISCRSSKSL |
| | | LHSNGITYLYWYLQKPGQSPQLLIYQMSNLVSGVPDRFSGS |
| | | GSGTDFTLKISRVEAEDVGVYYCAQNLELPYTFGGGTKVEIK |
| | | SGGGGSEIVLTQSPATLSLSPGERATLSCRASESVSSNLAWYQ |
| | | QKPGQAPRLLIYGAFNRATGIPARFSGSGSGTDFTLTISSLEPE |
| | | DFAVYYCQQRSDWFTFGGGTKVEIK |
| 36 | 5A3M5_scFv- | EVQLLESGGGLVQPGGSLRLSCAASGFTFSSYAMSWVRQAP |
| | Obi_LC | GKGLEWVSAISGSGGSTYYADSVKGRFTISRDNSKNTLYLQ |
| | | MNSLRAEDTAVYYCAKSYGAFDYVVGQGTLVTVSSGGGGSG |
| | | GGGSGGGGSDIQMTQSPSSLSASVGDRVTITCQASQDINKYL |
| | | NWYQQKPGKAPKLLIYGASRLETGVPSRFSGSGSGTDFTFTIS |
| | | SLQPEDIATYYCQQKHPRYPRTFGQGTKVEIKSGGGGSDIVM |
| | | TQTPLSLPVTPGEPASISCRSSKSLLHSNGITYLYWYLQKPGQ |
| | | SPQLLIYQMSNLVSGVPDRFSGSGSGTDFTLKISRVEAEDVGV |
| | | YYCAQNLELPYTFGGGTKVEIK |

CDR regions are in bold and linker is italicized.

TABLE 10
lists amino acid sequences of exemplary IgA Constant Regions

| SEQ | | |
|---|---|---|
| ID | | |
| NO: | Name | Constant Sequence |
| 37 | IgA3.0_constant_region | ASPTSPKVFPLSLDSTPQDGNVVVACLVQGFFPQEPLS |
| | | VTWSESGQGVTARNFPPSQDAS |
| | | GDLYTTSSQLTLPATQCPDGKSVTCHVKHYTNPSQDV |
| | | TVPCRVPPPPPCCHPRLSLHRPA |
| | | LEDLLLGSEANLTCTLTGLRDASGATFTWTPSSGKSA |
| | | VQGPPERDLCGCYSVSSVLPGSA |
| | | QPWNHGETFTCTAAHPELKTPLTATLSKSGNTFRPEV |
| | | HLLPPPSEELALNELVTLTCLAR |
| | | GFSPKDVLVRWLQGSQELPREKYLTWASRQEPSQGT |
| | | TTFAVTSILRVAAEDWKKGDTFSC |
| | | MVGHEALPLAFTQKTIDRLAGK |
| 38 | IgA2.0_constant region | ASPTSPKVFPLSLDSTPQDGNVVVACLVQGFFPQEPLS |
| | | VTWSESGQGVTARNFPPSQDASGDLYTTSSQLTLPAT |
| | | QCPDGKSVTCHVKHYTNPSQDVTVPCRVPPPPPCCHP |
| | | RLSLHRPALEDLLLGSEANLTCTLTGLRDASGATFTW |
| | | TPSSGKSAVQGPPERDLCGCYSVSSVLPGSAQPWNHG |
| | | ETFTCTAAHPELKTPLTATLSKSGNTFRPEVHLLPPPSE |
| | | ELALNELVTLTCLARGFSPKDVLVRWLQGSQELPREK |
| | | YLTWASRQEPSQGTTTFAVTSILRVAAEDWKKGDTFS |
| | | CMVGHEALPLAFTQKTIDRLAGKPTHVNVSVVMAEV |
| | | DGT |
| 39 | IgA2m1_constant region | ASPTSPKVFPLSLDSTPQDGNVVVACLVQGFFPQEPLS |
| | | VTWSESGQNVTARNFPPSQDASGDLYTTSSQLTLPAT |
| | | QCPDGKSVTCHVKHYTNPSQDVTVPCPVPPPPPCCHP |
| | | RLSLHRPALEDLLLGSEANLTCTLTGLRDASGATFTW |
| | | TPSSGKSAVQGPPERDLCGCYSVSSVLPGCAQPWNHG |
| | | ETFTCTAAHPELKTPLTANITKSGNTFRPEVHLLPPPSE |
| | | ELALNELVTLTCLARGFSPKDVLVRWLQGSQELPREK |
| | | YLTWASRQEPSQGTTTFAVTSILRVAAEDWKKGDTFS |
| | | CMVGHEALPLAFTQKTIDRLAGKPTHVNVSVVMAEV |
| | | DGTCY |
| 40 | IgA2m2_constant region | ASPTSPKVFPLSLDSTPQDGNVVVACLVQGFFPQEPLS |
| | | VTWSESGQNVTARNFPPSQDASGDLYTTSSQLTLPAT |
| | | QCPDGKSVTCHVKHYTNSSQDVTVPCRVPPPPPCCHP |
| | | RLSLHRPALEDLLLGSEANLTCTLTGLRDASGATFTW |
| | | TPSSGKSAVQGPPERDLCGCYSVSSVLPGCAQPWNHG |
| | | ETFTCTAAHPELKTPLTANITKSGNTFRPEVHLLPPPSE |
| | | ELALNELVTLTCLARGFSPKDVLVRWLQGSQELPREK |
| | | YLTWASRQEPSQGTTTYAVTSILRVAAEDWKKGETFS |
| | | CMVGHEALPLAFTQKTIDRLAGKPTHINVSVVMAEA |
| | | DGTCY |
| 41 | IgA1_constant region | ASPTSPKVFPLSLCSTQPDGNVVIACLVQGFFPQEPLS |
| | | VTWSESGQGVTARNFPPSQDASGDLYTTSSQLTLPAT |
| | | QCLAGKSVTCHVKHYTNPSQDVTVPCPVPSTPPTPSPS |
| | | TPPTPSPSCCHPRLSLHRPALEDLLLGSEANLTCTLTGL |
| | | RDASGVTFTWTPSSGKSAVQGPPERDLCGCYSVSSVL |
| | | PGCAEPWNHGKTFTCTAAYPESKTPLTATLSKSGNTF |
| | | RPEVHLLPPPSEELALNELVTLTCLARGFSPKDVLVR |
| | | WLQGSQELPREKYLTWASRQEPSQGTTTFAVTSILRV |
| | | AAEDWKKGDTFSCMVGHEALPLAFTQKTIDRLAGKP |
| | | THVNVSVVMAEVDGTCY |
| 42 | KappaLC_constant_region | RTVAAPSVFIFPPSDEQLKSGTASVVCLLNNFYPREAK |
| | | VQWKVDNALQSGNSQESVTEQDSKDSTYSLSSTLTLS |
| | | KADYEKHKVYACEVTHQGLSSPVTKSFNRGEC |
| 43 | LambdaLC_constant_ | GQPKAAPSVTLFPPSSEELQANKATLVCLISDFYPGAV |
| | region | TVAWKADSSPVKAGVETTTPSKQSNNKYAASSYLSL |
| | | TPEQWKSHRSYSCQVTHEGSTVEKTVAPTECS |

TABLE 11
lists amino acid sequences of linker peptides

| SEQ ID | | |
|---|---|---|
| NO: | Name | Amino acid sequence |
| 44 | Linker 1 | SGGGGS |
| 45 | Linker 2 | GGGGSGGGGSGGGGS |

TABLE 12
lists nucleotide sequences of the VH regions

| SEQ | | |
|---|---|---|
| ID | | |
| NO: | Name | VH |
| 46 | Obinituzumab_ | caggtgcagctgcaggaaagcggcccgggcctggtgaaaccgagcgg |
| | VH | caccctgagcctgacctgcgcggtgagcggcgtgagcattcgcagcatt |
| | | aactggtggaactgggtgcgccagccgccgggcaaaggcctggaatgg |
| | | attggcgaaatttatcatagcggcagcaccaactataacccgagcctgaa |
| | | aagccgcgtgaccattagcgtggataaaagcaaaaaccagtttagcctga |
| | | aactgaacagcgtgaccgcggcggataccgcggtgtattattgcgcgcg |
| | | cgatggcggcattgcggtgaccgattattattattatggcctggatgtgtgg |
| | | ggccagggcaccaccgtgaccgtgagcagc |
| 47 | 2.3D11_VH | caggtgcagctgcaggaaagcggcccgggcctggtgaaaccgagcgg |
| | | caccctgagcctgacctgcgcggtgagcggcgtgagcattcgcagcatt |
| | | aactggtggaactgggtgcgccagccgccgggcaaaggcctggaatgg |
| | | attggcgaaatttatcatagcggcagcaccaactataacccgagcctgaa |
| | | aagccgcgtgaccattagcgtggataaaagcaaaaaccagtttagcctga |
| | | aactgaacagcgtgaccgcggcggataccgcggtgtattattgcgcgcg |
| | | cgatggcggcattgcggtgaccgattattattattatggcctggatgtgtgg |
| | | ggccagggcaccaccgtgaccgtgagcagc |
| 48 | Common_VH | gaggttcagctgctggaatctggcggaggattggttcagcctggcggctc |
| | | tctgagactgtcttgtgccgcttccggcttcaccttctccagctacgctatgt |
| | | cctgggtccgacaggctcctggcaaaggactggaatgggtgtccgccat |
| | | ctctggctctggcggcagcacctactacgccgattctgtgaagggcagatt |
| | | caccatcagccgggacaactccaagaacaccctgtacctgcagatgaac |
| | | tccctgagagccgaggacaccgccgtgtactactgcgctaagtcttacgg |
| | | cgccttcgactattggggccagggcacactggtcaccgtgtcctct |

TABLE 13
lists nucleotide sequences of the VL regions

| SEQ ID | | |
|---|---|---|
| NO: | Name | VL |
| 49 | Obinituzumab_VL | Gacatcgtgatgacacagacacccctgagcctgcctgtga |
| | | cacctggcgagcctgcttccatctcctgccggtcctctaa |
| | | gtccctgctgcactctaacggcatcacctacctgtattgg |
| | | tacttgcagaagcctggccagtctcctcagctgctcatct |
| | | accagatgtccaacctggtgtctggcgtgcccgacagatt |
| | | ttctggctccggctccggaaccgatttcaccctgaagatc |
| | | tccagagtggaagccgaggacgtgggcgtgtactactgtg |
| | | cccagaacctggaactgccctacacctttggcggcggaac |
| | | aaaggtcgagatcaag |
| 50 | 2.3D11_VL | Gagatcgtgctgactcaatctcccgccacactgtctctga |
| | | gccctggcgaaagagctaccctgtcctgtagagcctccga |
| | | gtccgtgtcctctaacctggcctggtatcagcaaaaaccc |
| | | ggacaggccccacggctgttgatctacggcgccttcaata |
| | | gagccacaggcatccccgctagattctctggctccggctc |
| | | cggaacagactttacactgaccatctccagcctggaacct |
| | | gaggacttcgctgtgtactattgccagcagcggagcgact |
| | | ggttcaccttcggaggcggaacaaaggtcgagatcaag |
| 51 | 5A3M5_VL | Gacatccagatgacccagtctccatcctctctgtccgcct |
| | | ctgtgggcgacagagtgaccattacctgtcaggccagcca |
| | | ggacatcaacaagtacctgaactggtatcagcagaagccc |
| | | ggcaaggcccctaagctgttgatctacggcgcctctaggc |
| | | tggaaaccggcgtgccaagtagattctccggctctggctc |
| | | tggcaccgactttacctttacaatctccagcctgcagcct |
| | | gaggacattgccacctactactgccagcagaaacacccta |
| | | gataccctcggacctttggccagggcaccaaggtggaaat |
| | | caag |
| 52 | C2_VL | Aacttcatgctgacccagcctcactccgtgtctgagtctc |
| | | caggcaagaccgtgaccatctcttgcaccagatcctccgg |
| | | ctccatcgaggacaaatacgtgcagtggtatcagcagcgg |
| | | cctggctcctctcctaccatcgtgatctactacgacaacg |
| | | agcggccttctggcgtgcccgatagattctctggctctat |
| | | cgactcctcctccaactccgcctctctgacaatctccggc |
| | | ctgaaaaccgaggacgaggccgactactactgccagacct |
| | | acgaccagtctctgtacggctgggttttcggcggaggcac |
| | | caaactgacagtgctg |

TABLE 14
lists nucleotides sequences of DVD-IgA VHs

| SEQ | | |
|---|---|---|
| ID NO: | Name | Sequence |
| 53 | Obi_VH-linker- | Caggtgcagctggttcagtctggcgccgaagtgaagaa |
| | 2.3D11_VH | acctggctcctccgtgaaggtgtcctgcaaggcttctg |
| | | gctacgccttctcctactcctggatcaactgggtccga |
| | | caggctcctggacagggacttgagtggatgggcagaat |
| | | ctttcctggcgacggcgacaccgactacaacggcaagt |
| | | ttaagggcagagtgaccatcaccgccgacaagtctacc |
| | | tccaccgcctacatggaactgtccagcctgagatctga |
| | | ggacaccgccgtgtactactgcgccagaaacgtgttcg |
| | | acggctactggctggtgtattggggccagggaaccctg |
| | | gtcaccgtttcttctagcggaggcggaggatctcaggt |
| | | ccagctgcaagaatctggccctggcctggtcaagcctt |
| | | ctggcacactgtctctgacctgtgccgtgtctggcgtg |
| | | tccatccggtctatcaactggtggaattgggtccgcca |
| | | gcctccaggcaaaggcctggaatggatcggcgagatct |
| | | accactccggctccaccaactacaaccccagcctgaag |
| | | tcccgcgtgaccatctctgtggacaagtccaagaacca |
| | | gttctccctgaagctgaactccgtgaccgccgctgata |
| | | ccgctgtgtattactgtgctcgcgacggcggaatcgcc |
| | | gtgaccgattactactactacggcctggatgtgtgggg |
| | | acagggcaccacagtgacagtgtctagc |
| 54 | 5A3M5_VH- | Gaggtgcagctgctggaatctggcggaggattggttca |
| | linker-Obi_VH | gcctggcggctctctgagactgtcttgtgccgcttccg |
| | | gcttcaccttctccagctacgctatgtcctgggtccga |
| | | caggctcctggcaaaggactggaatgggtgtccgccat |
| | | ctctggctctggcggcagcacctactacgccgattctg |
| | | tgaagggcagattcaccatcagccgggacaactccaag |
| | | aacaccctgtacctgcagatgaactccctgagagccga |
| | | ggacaccgccgtgtactactgcgctaagtcttacggcg |
| | | ccttcgactattggggccagggcactctggtcaccgtt |
| | | tcttctagcggcggaggcggatctcaggttcagcttgt |
| | | tcagtctggcgccgaagtgaagaaacccggctctagcg |
| | | tgaaggtgtcctgcaaggcttctggctacgccttctcc |
| | | tactcctggatcaactgggttcgacaagcccctggaca |
| | | gggccttgagtggatgggcagaatctttcctggcgacg |
| | | gcgacaccgactacaacggcaagtttaagggccgcgtg |
| | | accatcaccgccgacaagtctacctctaccgcctacat |
| | | ggaactgtccagcctgcgctctgaggataccgctgtgt |
| | | attattgtgcccggaacgtgttcgacggctactggctg |
| | | gtttactggggacagggaaccctcgtgaccgtgtctag |
| | | C |
| 55 | Obi_VL-linker- | Gacatcgtgatgacccagacacctctgagcctgcctgt |
| | 2.3D11_VL | gacacctggcgagcctgcttccatctcctgccggtcct |
| | | ctaagtccctgctgcactctaacggcatcacctacctg |
| | | tactggtatctgcagaagcccggccagtctcctcagct |
| | | gctgatctaccagatgtccaacctggtgtctggcgtgc |
| | | ccgacagattttccggctctggctctggcaccgacttc |
| | | accctgaagatctccagagtggaagccgaggacgtggg |
| | | cgtgtactactgtgcccagaacctggaactgccctaca |
| | | cctttggcggaggcaccaaggtggaaatcaagtctggt |
| | | ggcggtggctccgagatcgtgctgactcaatctcccgc |
| | | cacactgtctctgagccctggcgaaagagctaccctgt |
| | | cctgtagagcctccgagtccgtgtcctctaacctggcc |
| | | tggtatcagcaaaaacccggacaggccccacggctgtt |
| | | gatctacggcgccttcaatagagccacaggcatccccg |
| | | ctagattctctggctccggctccggaacagactttaca |
| | | ctgaccatctccagcctggaacctgaggacttcgctgt |
| | | gtactattgccagcagcggagcgactggttcaccttcg |
| | | gaggcggaacaaaggtcgagatcaag |
| 56 | 5A3M5_VL- | gacatccagatgacccagtctccatcctctctgtccgc |
| | linker-Obi_VL | ctctgtgggcgacagagtgaccattacctgtcaggcca |
| | | gccaggacatcaacaagtacctgaactggtatcagcag |
| | | aagcccggcaaggcccctaagctgttgatctacggcgc |
| | | ctctagactggaaaccggcgtgccctctagattctccg |
| | | gctctggctctggcaccgactttacctttacaatctcc |
| | | agcctgcagcctgaggatatcgccacctactactgcca |
| | | gcagaaacaccctagataccctcggacctttggccagg |
| | | gcaccaaggtggaaatcaagtctggtggcggaggctcc |
| | | gacatcgtgatgacacagacacccctgagcctgcctgt |
| | | gacacctggcgagcctgcttccatctcctgccggtcct |
| | | ctaagtccctgctgcactctaacggcatcacctacctg |
| | | tattggtacttgcagaagcctggccagtctcctcagct |
| | | gctcatctaccagatgtccaacctggtgtctggcgtgc |
| | | ccgacagattttctggctccggctccggaaccgatttc |
| | | accctgaagatctccagagtggaagccgaggacgtggg |
| | | cgtgtactactgtgcccagaacctggaactgccctaca |
| | | cctttggcggcggaacaaaggtcgagatcaag |

TABLE 15
lists nucleotide sequences of scFv comprising
polypeptides (DVD-IgA scFv_HC)

| SEQ | | |
|---|---|---|
| ID | | |
| NO: | Name | Sequence |
| 57 | Obinituzumab_ | Caggtgcagctggttcagtctggcgccgaagtgaagaaacct |
| | scFv-2.3D11_HC | ggctcctccgtgaaggtgtcctgcaaggcttctggctacgcc |
| | | ttctcctactcctggatcaactgggtccgacaggctcctgga |
| | | cagggacttgagtggatgggcagaatctttcctggcgacggc |
| | | gacaccgactacaacggcaagtttaagggcagagtgaccatc |
| | | accgccgacaagtctacctccaccgcctacatggaactgtcc |
| | | agcctgagatctgaggacaccgccgtgtactactgcgccaga |
| | | aacgtgttcgacggctactggctggtgtattggggccaggga |
| | | acactggtcacagtgtctagcggaggcggaggatctggtggt |
| | | ggtggatctggcggcggaggctctgatatcgtgatgacccag |
| | | acacctctgagcctgcctgtgacacctggcgagcctgcttcc |
| | | atctcctgccggtcctctaagtccctgctgcactctaacggc |
| | | atcacctacctgtactggtatctgcagaagcccggccagtct |
| | | cctcagctgctgatctaccagatgtccaacctggtgtctggc |
| | | gtgcccgacagattttccggctctggctctggcaccgacttc |
| | | accctgaagatctccagagtggaagccgaggacgtgggcgtg |
| | | tactattgtgcccagaacctggaactgccctacacctttggc |
| | | ggaggcaccaaggtggaaatcaagagtggtggcggtggcagc |
| | | caggtccagctgcaagaatctggaccaggcctcgtgaagcct |
| | | agcggcacactgtctctgacctgtgctgtctctggcgtgtcc |
| | | atccggtccatcaactggtggaattgggtccgccagcctcca |
| | | ggcaaaggcctggaatggatcggcgagatctaccactccggc |
| | | tccaccaactacaaccccagcctgaagtcccgcgtgaccatc |
| | | tctgtggacaagtccaagaaccagttctccctgaagctgaac |
| | | tccgtgaccgccgctgataccgctgtgtattactgtgctcgc |
| | | gacggcggaatcgccgtgaccgattactactactacggcctg |
| | | gatgtgtggggacagggcaccacagtgactgtgtctagc |
| 58 | 5A3M5_scFv- | gaggtgcagctgctggaatctggcggaggattggttcagcct |
| | Obi_HC | ggcggctctctgagactgtcttgtgccgcttccggcttcacc |
| | | ttctccagctacgctatgtcctgggtccgacaggctcctggc |
| | | aaaggactggaatgggtgtccgccatctctggctctggcggc |
| | | agcacctactacgccgattctgtgaagggcagattcaccatc |
| | | agccgggacaactccaagaacaccctgtacctgcagatgaac |
| | | tccctgagagccgaggacaccgccgtgtactactgcgctaag |
| | | tcttacggcgccttcgactattggggccagggcacactggtc |
| | | acagtttctagcggcggaggtggaagcggaggcggaggtagt |
| | | ggtggtggcggatctgacatccagatgacccagtctccatcc |
| | | agcctgtctgcctctgtgggcgacagagtgaccatcacctgt |
| | | caggccagccaggacatcaacaagtacctgaactggtatcag |
| | | cagaagcccggcaaggcccctaagctgttgatctacggcgcc |
| | | agcagactggaaaccggcgtgccctctagattttccggctct |
| | | ggatctggcaccgactttacctttacaatctccagcctgcag |
| | | cctgaggatatcgctacctactactgccagcagaaacaccct |
| | | agataccctcggaccttcggacagggcaccaaggtggaaatc |
| | | aagtctggcggtggtggctcccaggttcagcttgttcaatct |
| | | ggcgccgaagtgaagaaacccggctccagtgtgaaggtgtcc |
| | | tgcaaggcttctggctacgccttctcctactcctggatcaac |
| | | tgggttcgacaagcccctggacagggccttgagtggatgggc |
| | | agaatctttcctggcgacggcgacaccgactacaacggcaag |
| | | tttaagggccgcgtgacaatcaccgccgacaagtctacctcc |
| | | accgcctacatggaactgtccagcctgagaagcgaggataca |
| | | gctgtgtattactgtgcccggaacgtgttcgacggctactgg |
| | | ctggtttactggggacagggaacactcgtgacagtgtctagc |

TABLE 16
lists nucleotide sequences of scFv comprising polypeptides
(DVD-IgA scFv_LC)

| SEQ | | |
|---|---|---|
| ID | | |
| NO: | Name | Sequence |
| 59 | Obinituzumab_ | Caggtgcagctggttcagtctggcgccgaagtgaagaaacctg |
| | scFv- | gctcctccgtgaaggtgtcctgcaaggcttctggctacgcctt |
| | 2.3D11_LC | ctcctactcctggatcaactgggtccgacaggctcctggacag |
| | | ggacttgagtggatgggcagaatctttcctggcgacggcgaca |
| | | ccgactacaacggcaagtttaagggcagagtgaccatcaccgc |
| | | cgacaagtctacctccaccgcctacatggaactgtccagcctg |
| | | agatctgaggacaccgccgtgtactactgcgccagaaacgtgt |
| | | tcgacggctactggctggtgtattggggccagggaacactggt |
| | | cacagtgtctagcggaggcggaggatctggtggtggtggatct |
| | | ggcggcggaggctctgatatcgtgatgacccagacacctctga |
| | | gcctgcctgtgacacctggcgagcctgcttccatctcctgccg |
| | | gtcctctaagtccctgctgcactctaacggcatcacctacctg |
| | | tactggtatctgcagaagcccggccagtctcctcagctgctga |
| | | tctaccagatgtccaacctggtgtctggcgtgcccgacagatt |
| | | ttccggctctggctctggcaccgacttcaccctgaagatctcc |
| | | agagtggaagccgaggacgtgggcgtgtactattgtgcccaga |
| | | acctggaactgccctacacctttggcggaggcaccaaggtgga |
| | | aatcaagagtggtggcggtggctccgagattgtgctgactcag |
| | | tctcccgccacactgtctttgagccctggcgagagagctaccc |
| | | tgtcctgtagagcctctgagtccgtgtcctctaacctggcctg |
| | | gtatcagcaaaaacccggacaggccccacggctgttgatctac |
| | | ggcgccttcaatagagccacaggcatccccgctagattctctg |
| | | gctccggctccggaacagactttacactgaccatctccagcct |
| | | ggaacctgaggatttcgctgtgtattactgccagcagcggagc |
| | | gactggttcacctttggaggcggaacaaaggtcgagatcaag |
| 60 | 5A3M5 scFv- | gaggtgcagctgctggaatctggcggaggattggttcagcctg |
| | Obi_LC | gcggctctctgagactgtcttgtgccgcttccggcttcacctt |
| | | ctccagctacgctatgtcctgggtccgacaggctcctggcaaa |
| | | ggactggaatgggtgtccgccatctctggctctggcggcagca |
| | | cctactacgccgattctgtgaagggcagattcaccatcagccg |
| | | ggacaactccaagaacaccctgtacctgcagatgaactccctg |
| | | agagccgaggacaccgccgtgtactactgcgctaagtcttacg |
| | | gcgccttcgactattggggccagggcacactggtcacagtttc |
| | | tagcggcggaggtggaagcggaggcggaggtagtggtggtggc |
| | | ggatctgacatccagatgacccagtctccatccagcctgtctg |
| | | cctctgtgggcgacagagtgaccatcacctgtcaggccagcca |
| | | ggacatcaacaagtacctgaactggtatcagcagaagcccggc |
| | | aaggcccctaagctgttgatctacggcgccagcagactggaaa |
| | | ccggcgtgccctctagattttccggctctggatctggcaccga |
| | | ctttacctttacaatctccagcctgcagcctgaggatatcgct |
| | | acctactactgccagcagaaacaccctagataccctcggacct |
| | | tcggacagggcaccaaggtggaaatcaagtctggcggtggcgg |
| | | ctccgatatcgtgatgacacagacccctctgagcctgcctgtg |
| | | acacctggcgagcctgcttccatctcctgccggtcctctaagt |
| | | ccctgctgcactctaacggcatcacctacctgtattggtactt |
| | | gcagaagcctggccagtctcctcagctgctcatctaccagatg |
| | | tccaacctggtgtctggcgtgcccgacagattttctggcagcg |
| | | gctctggcacagatttcaccctgaagatctccagagtggaagc |
| | | cgaggatgtgggcgtgtactattgtgcccagaacctggaactg |
| | | ccctacacctttggcggcggaacaaaggtcgagatcaag |

TABLE 17
lists sequences of immunoglobulin constant regions

| SEQ ID | | |
|---|---|---|
| NO: | Name | Constant Sequence |
| 61 | IgA3.0_constant_region_ | GCATCCCCGACCAGCCCCAAGGTCTTCCCGCT |
| | pEE14.4-vector | GAGCCTCGACAGCACCCCCCAAGATGGGAACG |
| | | TGGTCGTCGCATGCCTGGTCCAGGGCTTCTTC |
| | | CCCCAGGAGCCACTCAGTGTGACCTGGAGCGA |
| | | AAGCGGACAGGGTGTGACCGCCAGAAACTTCC |
| | | CACCTAGCCAGGATGCCTCCGGGGACCTGTAC |
| | | ACCACGAGCAGCCAGCTGACCCTGCCGGCCAC |
| | | ACAGTGCCCAGACGGCAAGTCCGTGACATGCC |
| | | ACGTGAAGCACTACACGAATCCCAGCCAGGAT |
| | | GTGACTGTGCCCTGCCGTGTTCCCCCACCTCC |
| | | CCCATGCTGCCACCCCCGACTGTCGCTGCACC |
| | | GACCGGCCCTCGAGGACCTGCTCTTAGGTTCA |
| | | GAAGCGAACCTCACGTGCACACTGACCGGCCT |
| | | GAGAGATGCCTCTGGTGCCACCTTCACCTGGA |
| | | CGCCCTCAAGTGGGAAGAGCGCTGTTCAAGGA |
| | | CCACCTGAGCGTGACCTCTGTGGCTGCTACAG |
| | | CGTGTCCAGTGTCCTGCCTGGCAGTGCCCAGC |
| | | CATGGAACCATGGGGAGACCTTCACCTGCACT |
| | | GCTGCCCACCCCGAGTTGAAGACCCCACTAAC |
| | | CGCCACTCTTAGTAAATCCGGAAACACATTCC |
| | | GGCCCGAGGTCCACCTGCTGCCGCCGCCGTCG |
| | | GAGGAGCTGGCCCTGAACGAGCTGGTGACGCT |
| | | GACGTGCCTGGCACGTGGCTTCAGCCCCAAGG |
| | | ATGTGCTGGTTCGCTGGCTGCAGGGGTCACAG |
| | | GAGCTGCCCCGCGAGAAGTACCTGACTTGGGC |
| | | ATCCCGGCAGGAGCCCAGCCAGGGCACCACCA |
| | | CCTTCGCTGTGACCAGCATACTGCGCGTGGCA |
| | | GCCGAGGACTGGAAGAAGGGGGACACCTTCTC |
| | | CTGCATGGTGGGCCACGAGGCCCTGCCGCTGG |
| | | CCTTCACACAGAAGACCATCGACCGCTTGGCG |
| | | GGTAAA |
| 62 | IgA3.0_constant_region- | gctagcccaacctctcctaaggtgttccctct |
| | pcDNA3.4 | gagcctggacagcacccctcaggatggaaatg |
| | | tggtggtggcctgtctggtgcagggattcttc |
| | | ccacaagagcccctgtccgtgacttggagcga |
| | | atctggacagggcgtgaccgccagaaacttcc |
| | | caccttctcaggacgcctctggcgacctgtac |
| | | accacctcttctcagctgaccctgcctgccac |
| | | acagtgccctgatggcaagtctgtgacctgcc |
| | | acgtgaagcactacaccaatcctagccaggac |
| | | gtgaccgtgccttgcagagttcctcctcctcc |
| | | accttgctgtcaccctcggctgtctctgcaca |
| | | gacccgctctggaagatctgctgctgggctct |
| | | gaggccaacctgacatgtaccctgaccggcct |
| | | gagagatgcttctggcgccacctttacctgga |
| | | caccttccagcggaaagtccgctgttcaggga |
| | | cctcctgagagggacctgtgcggctgttactc |
| | | tgtgtctagtgtgctgcctggcagcgcccagc |
| | | cttggaatcatggcgagacattcacctgtacc |
| | | gctgctcaccccgagctgaaaacccctctgac |
| | | cgccacactgtccaagtccggcaacaccttcc |
| | | ggcctgaagtgcatctgctgcctccacctagc |
| | | gaggaactggccctgaatgagctggtcaccct |
| | | gacctgtctggccaggggctttagccctaagg |
| | | acgtgctcgttagatggctgcagggctcccaa |
| | | gagctgcccagagagaagtatctgacctgggc |
| | | ctctcggcaagagccatctcagggcaccacaa |
| | | cctttgccgtgaccagcatcctgagagtggcc |
| | | gccgaagattggaagaagggcgacaccttcag |
| | | ctgcatggtcggacatgaagccctgcctctgg |
| | | ctttcacccagaaaaccatcgacagactggcc |
| | | ggcaag |
| 63 | IgA2.0_constant region | GCATCCCCGACCAGCCCCAAGGTCTTCCCGCT |
| | | GAGCCTCGACAGCACCCCCCAAGATGGGAACG |
| | | TGGTCGTCGCATGCCTGGTCCAGGGCTTCTTC |
| | | CCCCAGGAGCCACTCAGTGTGACCTGGAGCGA |
| | | AAGCGGACAGGGTGTGACCGCCAGAAACTTCC |
| | | CACCTAGCCAGGATGCCTCCGGGGACCTGTAC |
| | | ACCACGAGCAGCCAGCTGACCCTGCCGGCCAC |
| | | ACAGTGCCCAGACGGCAAGTCCGTGACATGCC |
| | | ACGTGAAGCACTACACGAATCCCAGCCAGGAT |
| | | GTGACTGTGCCCTGCCGTGTTCCCCCACCTCC |
| | | CCCATGCTGCCACCCCCGACTGTCGCTGCACC |
| | | GACCGGCCCTCGAGGACCTGCTCTTAGGTTCA |
| | | GAAGCGAACCTCACGTGCACACTGACCGGCCT |
| | | GAGAGATGCCTCTGGTGCCACCTTCACCTGGA |
| | | CGCCCTCAAGTGGGAAGAGCGCTGTTCAAGGA |
| | | CCACCTGAGCGTGACCTCTGTGGCTGCTACAG |
| | | CGTGTCCAGTGTCCTGCCTGGCAGTGCCCAGC |
| | | CATGGAACCATGGGGAGACCTTCACCTGCACT |
| | | GCTGCCCACCCCGAGTTGAAGACCCCACTAAC |
| | | CGCCACTCTTAGTAAATCCGGAAACACATTCC |
| | | GGCCCGAGGTCCACCTGCTGCCGCCGCCGTCG |
| | | GAGGAGCTGGCCCTGAACGAGCTGGTGACGCT |
| | | GACGTGCCTGGCACGTGGCTTCAGCCCCAAGG |
| | | ATGTGCTGGTTCGCTGGCTGCAGGGGTCACAG |
| | | GAGCTGCCCCGCGAGAAGTACCTGACTTGGGC |
| | | ATCCCGGCAGGAGCCCAGCCAGGGCACCACCA |
| | | CCTTCGCTGTGACCAGCATACTGCGCGTGGCA |
| | | GCCGAGGACTGGAAGAAGGGGGACACCTTCTC |
| | | CTGCATGGTGGGCCACGAGGCCCTGCCGCTGG |
| | | CCTTCACACAGAAGACCATCGACCGCTTGGCG |
| | | GGTAAACCCACCCATGTCAATGTGTCTGTTGT |
| | | CATGGCGGAGGTGGACGGCACC |
| 64 | IgA2m1_constant region | GCATCCCCGACCAGCCCCAAGGTCTTCCCGCT |
| | | GAGCCTCGACAGCACCCCCCAAGATGGGAACG |
| | | TGGTCGTCGCATGCCTGGTCCAGGGCTTCTTC |
| | | CCCCAGGAGCCACTCAGTGTGACCTGGAGCGA |
| | | AAGCGGACAGAACGTGACCGCCAGAAACTTCC |
| | | CACCTAGCCAGGATGCCTCCGGGGACCTGTAC |
| | | ACCACGAGCAGCCAGCTGACCCTGCCGGCCAC |
| | | ACAGTGCCCAGACGGCAAGTCCGTGACATGCC |
| | | ACGTGAAGCACTACACGAATCCCAGCCAGGAT |
| | | GTGACTGTGCCCTGCCCAGTTCCCCCACCTCC |
| | | CCCATGCTGCCACCCCCGACTGTCGCTGCACC |
| | | GACCGGCCCTCGAGGACCTGCTCTTAGGTTCA |
| | | GAAGCGAACCTCACGTGCACACTGACCGGCCT |
| | | GAGAGATGCCTCTGGTGCCACCTTCACCTGGA |
| | | CGCCCTCAAGTGGGAAGAGCGCTGTTCAAGGA |
| | | CCACCTGAGCGTGACCTCTGTGGCTGCTACAG |
| | | CGTGTCCAGTGTCCTGCCTGGCTGTGCCCAGC |
| | | CATGGAACCATGGGGAGACCTTCACCTGCACT |
| | | GCTGCCCACCCCGAGTTGAAGACCCCACTAAC |
| | | CGCCAACATCACAAAATCCGGAAACACATTCC |
| | | GGCCCGAGGTCCACCTGCTGCCGCCGCCGTCG |
| | | GAGGAGCTGGCCCTGAACGAGCTGGTGACGCT |
| | | GACGTGCCTGGCACGTGGCTTCAGCCCCAAGG |
| | | ATGTGCTGGTTCGCTGGCTGCAGGGGTCACAG |
| | | GAGCTGCCCCGCGAGAAGTACCTGACTTGGGC |
| | | ATCCCGGCAGGAGCCCAGCCAGGGCACCACCA |
| | | CCTTCGCTGTGACCAGCATACTGCGCGTGGCA |
| | | GCCGAGGACTGGAAGAAGGGGGACACCTTCTC |
| | | CTGCATGGTGGGCCACGAGGCCCTGCCGCTGG |
| | | CCTTCACACAGAAGACCATCGACCGCTTGGCG |
| | | GGTAAACCCACCCATGTCAATGTGTCTGTTGT |
| | | CATGGCGGAGGTGGACGGCACCTGCTAC |
| 65 | IgA2m2_constant region | gccagccccaccagccccaaggtgttccccct |
| | | gagcctggacagcaccccccaggacggcaacg |
| | | tggtggtggcctgcctggtgcagggcttcttc |
| | | ccccaggagcccctgagcgtgacctggagcga |
| | | gagcggccagaacgtgaccgccagaaacttcc |
| | | cccccagccaggacgccagcggcgacctgtac |
| | | accaccagcagccagctgaccctgcccgccac |
| | | ccagtgccccgacggcaagagcgtgacctgcc |
| | | acgtgaagcactacaccaacagcagccaggac |
| | | gtgaccgtgccctgcagagtgccccccccccc |
| | | cccctgctgccaccccagactgagcctgcaca |
| | | gacccgccctggaggacctgctgctgggcagc |
| | | gaggccaacctgacctgcaccctgaccggcct |
| | | gagagacgccagcggcgccaccttcacctgga |
| | | cccccagcagcggcaagagcgccgtgcagggc |
| | | ccccccgagagagacctgtgcggctgctacag |
| | | cgtgagcagcgtgctgcccggctgcgcccagc |
| | | cctggaaccacggcgagaccttcacctgcacc |
| | | gccgcccaccccgagctgaagacccccctgac |
| | | cgccaacatcaccaagagcggcaacaccttca |
| | | gacccgaggtgcacctgctgcccccccccagc |
| | | gaggagctggccctgaacgagctggtgaccct |
| | | gacctgcctggccagaggcttcagccccaagg |
| | | acgtgctggtgagatggctgcagggcagccag |
| | | gagctgcccagagagaagtacctgacctgggc |
| | | cagcagacaggagcccagccagggcaccacca |
| | | cctacgccgtgaccagcatcctgagagtggcc |
| | | gccgaggactggaagaagggcgagaccttcag |
| | | ctgcatggtgggccacgaggccctgcccctgg |
| | | ccttcacccagaagaccatcgacagactggcc |
| | | ggcaagcccacccacatcaacgtgagcgtggt |
| | | gatggccgaggccgacggcacctgctac |
| 66 | IgA1_constant region | GCATCCCCGACCAGCCCCAAGGTCTTC |
| | | CCGCTGAGCCTCTGCAGCACCCAGCCAGATGG |
| | | GAACGTGGTCATCGCCTGCCTGGTCCAGGGCT |
| | | TCTTCCCCCAGGAGCCACTCAGTGTGACCTGG |
| | | AGCGAAAGCGGACAGGGCGTGACCGCCAGAAA |
| | | CTTCCCACCCAGCCAGGATGCCTCCGGGGACC |
| | | TGTACACCACGAGCAGCCAGCTGACCCTGCCG |
| | | GCCACACAGTGCCTAGCCGGCAAGTCCGTGAC |
| | | ATGCCACGTGAAGCACTACACGAATCCCAGCC |
| | | AGGATGTGACTGTGCCCTGCCCAGTTCCCTCA |
| | | ACTCCACCTACCCCATCTCCCTCAACTCCACC |
| | | TACCCCATCTCCCTCATGCTGCCACCCCCGAC |
| | | TGTCACTGCACCGACCGGCCCTCGAGGACCTG |
| | | CTCTTAGGTTCAGAAGCGAACCTCACGTGCAC |
| | | ACTGACCGGCCTGAGAGATGCCTCAGGTGTCA |
| | | CCTTCACCTGGACGCCCTCAAGTGGGAAGAGC |
| | | GCTGTTCAAGGACCACCTGACCGTGACCTCTG |
| | | TGGCTGCTACAGCGTGTCCAGTGTCCTGCCGG |
| | | GCTGTGCCGAGCCATGGAACCATGGGAAGACC |
| | | TTCACTTGCACTGCTGCCTACCCCGAGTCCAA |
| | | GACCCCGCTAACCGCCACCCTCTCAAAATCCG |
| | | GAAACACATTCCGGCCCGAGGTCCACCTGCTG |
| | | CCGCCGCCGTCGGAGGAGCTGGCCCTGAACGA |
| | | GCTGGTGACGCTGACGTGCCTGGCACGTGGCT |
| | | TCAGCCCCAAGGATGTGCTGGTTCGCTGGCTG |
| | | CAGGGGTCACAGGAGCTGCCCCGCGAGAAGTA |
| | | CCTGACTTGGGCATCCCGGCAGGAGCCCAGCC |
| | | AGGGCACCACCACCTTCGCTGTGACCAGCATA |
| | | CTGCGCGTGGCAGCCGAGGACTGGAAGAAGGG |
| | | GGACACCTTCTCCTGCATGGTGGGCCACGAGG |
| | | CCCTGCCGCTGGCCTTCACACAGAAGACCATC |
| | | GACCGCTTGGCGGGTAAACCCACCCATGTCAA |
| | | TGTGTCTGTTGTCATGGCGGAGGTGGACGGCA |
| | | CCTGCTAC |
| 67 | KappaLC_constant_ | CGAACTGTGGCTGCACCATCTGTCTTCATCTT |
| | region_pEE14.4-vector | CCCGCCATCTGATGAGCAGTTGAAATCTGGAA |
| | | CTGCCTCTGTTGTGTGCCTGCTGAATAACTTC |
| | | TATCCCAGAGAGGCCAAAGTACAGTGGAAGGT |
| | | GGATAACGCCCTCCAATCGGGTAACTCCCAGG |
| | | AGAGTGTCACAGAGCAGGACAGCAAGGACAGC |
| | | ACCTACAGCCTCAGCAGCACCCTGACGCTGAG |
| | | CAAAGCAGACTACGAGAAACACAAAGTCTACG |
| | | CCTGCGAAGTCACCCATCAGGGCCTGAGCTCG |
| | | CCCGTCACAAAGAGCTTCAACAGGGGAGAGTG |
| | | T |
| 68 | KappaLC_constant_ | Cggacagtggccgctccttccgtgttcatctt |
| | region-pcDNA3.4 | cccaccttccgacgagcagctgaagtccggca |
| | | cagctagcgtggtctgcctgctgaacaacttc |
| | | taccctcgggaagccaaggtgcagtggaaggt |
| | | ggacaatgccctgcagtccggcaactcccaag |
| | | agtctgtgaccgagcaggactccaaggacagc |
| | | acctacagcctgtcctccacactgaccctgtc |
| | | caaggccgactacgagaagcacaaggtgtacg |
| | | cctgcgaagtgacccatcagggcctgtctagc |
| | | cctgtgaccaagtctttcaaccggggcgagtg |
| | | t |
| 69 | LambdaLC_constant_ | ggacagcctaaggccgctccatccgtgacact |
| | region | gttccctccatcctccgaggaactgcaggcca |
| | | acaaggctaccctcgtgtgcctgatctccgac |
| | | ttttaccctggcgctgtgaccgtggcctggaa |
| | | ggctgatagttctcctgtgaaggccggcgtgg |
| | | aaaccaccacaccttccaagcagtccaacaac |
| | | aaatacgccgctagctcctacctgtctctgac |
| | | ccctgaacagtggaagtcccaccggtcctaca |
| | | gctgccaagtgacccatgagggctccaccgtg |
| | | gaaaagaccgtggctcctaccgagtgctct |

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments described herein, or combinations of one or more of these embodiments or aspects described therein may be employed in practicing the present disclosure. It is intended that the following claims define the scope of the present disclosure and that methods and structures within the scope of these claims and their equivalents be covered thereby.

What is claimed is:

1. An antibody construct comprising;
(a) an immunoglobulin A (IgA) heavy chain domain;
(b) a CD47 binding domain; and
(c) an antigen binding domain;
wherein the IgA heavy chain domain specifically binds a FcαR on an immune effector cell,
wherein the CD47 binding domain inhibits binding of a CD47 expressed on a target cell with a signal regulatory protein α (SIRP α) on the immune effector cell,
wherein the antigen binding domain binds an antigen on the target cell, and
wherein the antibody construct has a higher binding affinity for the antigen compared to the CD47.


SEQUENCE LISTING (149 sequences)

SEQ ID NO: 1 - protein, 7 aa, Artificial Sequence
  Description of Artificial Sequence Synthetic peptide
GYAFSYS

SEQ ID NO: 2 - protein, 13 aa, Artificial Sequence
  Description of Artificial Sequence Synthetic peptide
RIFPGDGDTDYNG

SEQ ID NO: 3 - protein, 10 aa, Artificial Sequence
  Description of Artificial Sequence Synthetic peptide
NVFDGYWLVY

SEQ ID NO: 4 - protein, 8 aa, Artificial Sequence
  Description of Artificial Sequence Synthetic peptide
GVSIRSIN

SEQ ID NO: 5 - protein, 16 aa, Artificial Sequence
  Description of Artificial Sequence Synthetic peptide
EIYHSGSTNYNPSLKS

SEQ ID NO: 6 - protein, 16 aa, Artificial Sequence
  Description of Artificial Sequence Synthetic peptide
DGGIAVTDYYYYGLDV

SEQ ID NO: 7 - protein, 10 aa, Artificial Sequence
  Description of Artificial Sequence Synthetic peptide
GFTFSSYAMS

SEQ ID NO: 8 - protein, 17 aa, Artificial Sequence
  Description of Artificial Sequence Synthetic peptide
AISGSGGSTYYADSVKG

SEQ ID NO: 9 - protein, 7 aa, Artificial Sequence
  Description of Artificial Sequence Synthetic peptide
SYGAFDY

SEQ ID NO: 10 - protein, 16 aa, Artificial Sequence
  Description of Artificial Sequence Synthetic peptide
RSSKSLLHSNGITYLY

SEQ ID NO: 11 - protein, 7 aa, Artificial Sequence
  Description of Artificial Sequence Synthetic peptide
QMSNLVS

SEQ ID NO: 12 - protein, 9 aa, Artificial Sequence
  Description of Artificial Sequence Synthetic peptide
AQNLELPYT

SEQ ID NO: 13 - protein, 11 aa, Artificial Sequence
  Description of Artificial Sequence Synthetic peptide
RASESVSSNLA

SEQ ID NO: 14 - protein, 7 aa, Artificial Sequence
  Description of Artificial Sequence Synthetic peptide
GAFNRAT

SEQ ID NO: 15 - protein, 8 aa, Artificial Sequence
  Description of Artificial Sequence Synthetic peptide
QQRSDWFT

SEQ ID NO: 16 - protein, 11 aa, Artificial Sequence
  Description of Artificial Sequence Synthetic peptide
QASQDINKYLN

SEQ ID NO: 17 - protein, 7 aa, Artificial Sequence
  Description of Artificial Sequence Synthetic peptide
GASRLET

SEQ ID NO: 18 - protein, 10 aa, Artificial Sequence
  Description of Artificial Sequence Synthetic peptide
QQKHPRYPRT

SEQ ID NO: 19 - protein, 13 aa, Artificial Sequence
  Description of Artificial Sequence Synthetic peptide
TRSSGSIEDKYVQ

SEQ ID NO: 20 - protein, 7 aa, Artificial Sequence
  Description of Artificial Sequence Synthetic peptide
YDNERPS

SEQ ID NO: 21 - protein, 11 aa, Artificial Sequence
  Description of Artificial Sequence Synthetic peptide
QTYDQSLYGWV

SEQ ID NO: 22 - protein, 119 aa, Artificial Sequence
  Description of Artificial Sequence Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 23 - protein, 125 aa, Artificial Sequence
  Description of Artificial Sequence Synthetic polypeptide
QVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSS

SEQ ID NO: 24 - protein, 116 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 25 - protein, 112 aa, Artificial Sequence
  Description of Artificial Sequence Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 26 - protein, 106 aa, Artificial Sequence
  Description of Artificial Sequence Synthetic polypeptide
EIVLTQSPATLSLSPGERATLSCRASESVSSNLAWYQQKPGQAPRLLIYGAFNRATGIPARFSGSGSGTDFTLTISSLEPEDFAVYYCQQRSDWFTFGGGTKVEIK

SEQ ID NO: 27 - protein, 108 aa, Artificial Sequence
  Description of Artificial Sequence Synthetic polypeptide
DIQMTQSPSSLSASVGDRVTITCQASQDINKYLNWYQQKPGKAPKLLIYGASRLETGVPSRFSGSGSGTDFTFTISSLQPEDIATYYCQQKHPRYPRTFGQGTKVEIK

SEQ ID NO: 28 - protein, 112 aa, Artificial Sequence
  Description of Artificial Sequence Synthetic polypeptide
NFMLTQPHSVSESPGKTVTISCTRSSGSIEDKYVQWYQQRPGSSPTIVIYYDNERPSGVPDRFSGSIDSSSNSASLTISGLKTEDEADYYCQTYDQSLYGWVFGGGTKLTVL

SEQ ID NO: 29 - protein, 250 aa, Artificial Sequence
  Description of Artificial Sequence Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSSGGGGSQVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSS

SEQ ID NO: 30 - protein, 241 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSSGGGGSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 31 - protein, 224 aa, Artificial Sequence
  Description of Artificial Sequence Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKSGGGGSEIVLTQSPATLSLSPGERATLSCRASESVSSNLAWYQQKPGQAPRLLIYGAFNRATGIPARFSGSGSGTDFTLTISSLEPEDFAVYYCQQRSDWFTFGGGTKVEIK

SEQ ID NO: 32 - protein, 226 aa, Artificial Sequence
  Description of Artificial Sequence Synthetic polypeptide
DIQMTQSPSSLSASVGDRVTITCQASQDINKYLNWYQQKPGKAPKLLIYGASRLETGVPSRFSGSGSGTDFTFTISSLQPEDIATYYCQQKHPRYPRTFGQGTKVEIKSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 33 - protein, 377 aa, Artificial Sequence
  Description of Artificial Sequence Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSGGGGSGGGGSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKSGGGGSQVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSS

SEQ ID NO: 34 - protein, 364 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSGGGGSGGGGSGGGGSDIQMTQSPSSLSASVGDRVTITCQASQDINKYLNWYQQKPGKAPKLLIYGASRLETGVPSRFSGSGSGTDFTFTISSLQPEDIATYYCQQKHPRYPRTFGQGTKVEIKSGGGGSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 35 - protein, 358 aa, Artificial Sequence
  Description of Artificial Sequence Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSGGGGSGGGGSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKSGGGGSEIVLTQSPATLSLSPGERATLSCRASESVSSNLAWYQQKPGQAPRLLIYGAFNRATGIPARFSGSGSGTDFTLTISSLEPEDFAVYYCQQRSDWFTFGGGTKVEIK

SEQ ID NO: 36 - protein, 357 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSGGGGSGGGGSGGGGSDIQMTQSPSSLSASVGDRVTITCQASQDINKYLNWYQQKPGKAPKLLIYGASRLETGVPSRFSGSGSGTDFTFTISSLQPEDIATYYCQQKHPRYPRTFGQGTKVEIKSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 37 - protein, 322 aa, Artificial Sequence
  Description of Artificial Sequence Synthetic polypeptide
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 38 - protein, 338 aa, Artificial Sequence
  Description of Artificial Sequence Synthetic polypeptide
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGT

SEQ ID NO: 39 - protein, 340 aa, Artificial Sequence
  Description of Artificial Sequence Synthetic polypeptide
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 40 - protein, 340 aa, Artificial Sequence
  Description of Artificial Sequence Synthetic polypeptide
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRLAGKPTHINVSVVMAEADGTCY

SEQ ID NO: 41 - protein, 353 aa, Artificial Sequence
  Description of Artificial Sequence Synthetic polypeptide
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 42 - protein, 107 aa, Artificial Sequence [sequence omitted]

SEQ ID NO: 43 - protein, 106 aa, Artificial Sequence
  Description of Artificial Sequence Synthetic polypeptide
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - protein, 6 aa, Artificial Sequence
  Description of Artificial Sequence Synthetic peptide
SGGGGS

SEQ ID NO: 45 - protein, 15 aa, Artificial Sequence
  Description of Artificial Sequence Synthetic peptide
GGGGSGGGGSGGGGS

SEQ ID NO: 46 - DNA, 375 bp, Artificial Sequence
  Description of Artificial Sequence Synthetic polynucleotide
caggtgcagctgcaggaaagcggcccgggcctggtgaaaccgagcggcaccctgagcctgacctgcgcggtgagcggcgtgagcattcgcagcattaactggtggaactgggtgcgccagccgccgggcaaaggcctggaatggattggcgaaatttatcatagcggcagcaccaactataacccgagcctgaaaagccgcgtgaccattagcgtggataaaagcaaaaaccagtttagcctgaaactgaacagcgtgaccgcggcggataccgcggtgtattattgcgcgcgcgatggcggcattgcggtgaccgattattattattatggcctggatgtgtggggccagggcaccaccgtgaccgtgagcagc

SEQ ID NO: 47 - DNA, 375 bp, Artificial Sequence; identical to SEQ ID NO: 46
  Description of Artificial Sequence Synthetic polynucleotide

SEQ ID NO: 48 - DNA, 348 bp, Artificial Sequence
  Description of Artificial Sequence Synthetic polynucleotide
gaggttcagctgctggaatctggcggaggattggttcagcctggcggctctctgagactgtcttgtgccgcttccggcttcaccttctccagctacgctatgtcctgggtccgacaggctcctggcaaaggactggaatgggtgtccgccatctctggctctggcggcagcacctactacgccgattctgtgaagggcagattcaccatcagccgggacaactccaagaacaccctgtacctgcagatgaactccctgagagccgaggacaccgccgtgtactactgcgctaagtcttacggcgccttcgactattggggccagggcacactggtcaccgtgtcctct

SEQ ID NO: 49 - DNA, 336 bp, Artificial Sequence
  Description of Artificial Sequence Synthetic polynucleotide
gacatcgtgatgacacagacacccctgagcctgcctgtgacacctggcgagcctgcttccatctcctgccggtcctctaagtccctgctgcactctaacggcatcacctacctgtattggtacttgcagaagcctggccagtctcctcagctgctcatctaccagatgtccaacctggtgtctggcgtgcccgacagattttctggctccggctccggaaccgatttcaccctgaagatctccagagtggaagccgaggacgtgggcgtgtactactgtgcccagaacctggaactgccctacacctttggcggcggaacaaaggtcgagatcaag

SEQ ID NO: 50 - DNA, 318 bp, Artificial Sequence
  Description of Artificial Sequence Synthetic polynucleotide
gagatcgtgctgactcaatctcccgccacactgtctctgagccctggcgaaagagctaccctgtcctgtagagcctccgagtccgtgtcctctaacctggcctggtatcagcaaaaacccggacaggccccacggctgttgatctacggcgccttcaatagagccacaggcatccccgctagattctctggctccggctccggaacagactttacactgaccatctccagcctggaacctgaggacttcgctgtgtactattgccagcagcggagcgactggttcaccttcggaggcggaacaaaggtcgagatcaag

SEQ ID NO: 51 - DNA, 324 bp, Artificial Sequence
  Description of Artificial Sequence Synthetic polynucleotide
gacatccagatgacccagtctccatcctctctgtccgcctctgtgggcgacagagtgaccattacctgtcaggccagccaggacatcaacaagtacctgaactggtatcagcagaagcccggcaaggcccctaagctgttgatctacggcgcctctaggctggaaaccggcgtgccaagtagattctccggctctggctctggcaccgactttacctttacaatctccagcctgcagcctgaggacattgccacctactactgccagcagaaacaccctagataccctcggacctttggccagggcaccaaggtggaaatcaag

SEQ ID NO: 52 - DNA, 336 bp, Artificial Sequence
  Description of Artificial Sequence Synthetic polynucleotide
aacttcatgctgacccagcctcactccgtgtctgagtctccaggcaagaccgtgaccatctcttgcaccagatcctccggctccatcgaggacaaatacgtgcagtggtatcagcagcggcctggctcctctcctaccatcgtgatctactacgacaacgagcggccttctggcgtgcccgatagattctctggctctatcgactcctcctccaactccgcctctctgacaatctccggcctgaaaaccgaggacgaggccgactactactgccagacctacgaccagtctctgtacggctgggttttcggcggaggcaccaaactgacagtgctg

SEQ ID NO: 53 - DNA, 750 bp, Artificial Sequence
  Description of Artificial Sequence Synthetic polynucleotide
caggtgcagctggttcagtctggcgccgaagtgaagaaacctggctcctccgtgaaggtgtcctgcaaggcttctggctacgccttctcctactcctggatcaactgggtccgacaggctcctggacagggacttgagtggatgggcagaatctttcctggcgacggcgacaccgactacaacggcaagtttaagggcagagtgaccatcaccgccgacaagtctacctccaccgcctacatggaactgtccagcctgagatctgaggacaccgccgtgtactactgcgccagaaacgtgttcgacggctactggctggtgtattggggccagggaaccctggtcaccgtttcttctagcggaggcggaggatctcaggtccagctgcaagaatctggccctggcctggtcaagccttctggcacactgtctctgacctgtgccgtgtctggcgtgtccatccggtctatcaactggtggaattgggtccgccagcctccaggcaaaggcctggaatggatcggcgagatctaccactccggctccaccaactacaaccccagcctgaagtcccgcgtgaccatctctgtggacaagtccaagaaccagttctccctgaagctgaactccgtgaccgccgctgataccgctgtgtattactgtgctcgcgacggcggaatcgccgtgaccgattactactactacggcctggatgtgtggggacagggcaccacagtgacagtgtctagc

SEQ ID NO: 54 - DNA, 723 bp, Artificial Sequence
  Description of Artificial Sequence Synthetic polynucleotide
gaggtgcagctgctggaatctggcggaggattggttcagcctggcggctctctgagactgtcttgtgccgcttccggcttcaccttctccagctacgctatgtcctgggtccgacaggctcctggcaaaggactggaatgggtgtccgccatctctggctctggcggcagcacctactacgccgattctgtgaagggcagattcaccatcagccgggacaactccaagaacaccctgtacctgcagatgaactccctgagagccgaggacaccgccgtgtactactgcgctaagtcttacggcgccttcgactattggggccagggcactctggtcaccgtttcttctagcggcggaggcggatctcaggttcagcttgttcagtctggcgccgaagtgaagaaacccggctctagcgtgaaggtgtcctgcaaggcttctggctacgccttctcctactcctggatcaactgggttcgacaagcccctggacagggccttgagtggatgggcagaatctttcctggcgacggcgacaccgactacaacggcaagtttaagggccgcgtgaccatcaccgccgacaagtctacctctaccgcctacatggaactgtccagcctgcgctctgaggataccgctgtgtattattgtgcccggaacgtgttcgacggctactggctggtttactggggacagggaaccctcgtgaccgtgtctagc

SEQ ID NO: 55 - DNA, 672 bp, Artificial Sequence
  Description of Artificial Sequence Synthetic polynucleotide
gacatcgtgatgacccagacacctctgagcctgcctgtgacacctggcgagcctgcttccatctcctgccggtcctctaagtccctgctgcactctaacggcatcacctacctgtactggtatctgcagaagcccggccagtctcctcagctgctgatctaccagatgtccaacctggtgtctggcgtgcccgacagattttccggctctggctctggcaccgacttcaccctgaagatctccagagtggaagccgaggacgtgggcgtgtactactgtgcccagaacctggaactgccctacacctttggcggaggcaccaaggtggaaatcaagtctggtggcggtggctccgagatcgtgctgactcaatctcccgccacactgtctctgagccctggcgaaagagctaccctgtcctgtagagcctccgagtccgtgtcctctaacctggcctggtatcagcaaaaacccggacaggccccacggctgttgatctacggcgccttcaatagagccacaggcatccccgctagattctctggctccggctccggaacagactttacactgaccatctccagcctggaacctgaggacttcgctgtgtactattgccagcagcggagcgactggttcaccttcggaggcggaacaaaggtcgagatcaag

SEQ ID NO: 56 - DNA, 678 bp, Artificial Sequence
  Description of Artificial Sequence Synthetic polynucleotide
gacatccagatgacccagtctccatcctctctgtccgcctctgtgggcgacagagtgaccattacctgtcaggccagccaggacatcaacaagtacctgaactggtatcagcagaagcccggcaaggcccctaagctgttgatctacggcgcctctagactggaaaccggcgtgccctctagattctccggctctggctctggcaccgactttacctttacaatctccagcctgcagcctgaggatatcgccacctactactgccagcagaaacaccctagataccctcggacctttggccagggcaccaaggtggaaatcaagtctggtggcggaggctccgacatcgtgatgacacagacacccctgagcctgcctgtgacacctggcgagcctgcttccatctcctgccggtcctctaagtccctgctgcactctaacggcatcacctacctgtattggtacttgcagaagcctggccagtctcctcagctgctcatctaccagatgtccaacctggtgtctggcgtgcccgacagattttctggctccggctccggaaccgatttcaccctgaagatctccagagtggaagccgaggacgtgggcgtgtactactgtgcccagaacctggaactgccctacacctttggcggcggaacaaaggtcgagatcaag

SEQ ID NO: 57 - DNA, 1131 bp, Artificial Sequence
  Description of Artificial Sequence Synthetic polynucleotide
caggtgcagctggttcagtctggcgccgaagtgaagaaacctggctcctccgtgaaggtgtcctgcaaggcttctggctacgccttctcctactcctggatcaactgggtccgacaggctcctggacagggacttgagtggatgggcagaatctttcctggcgacggcgacaccgactacaacggcaagtttaagggcagagtgaccatcaccgccgacaagtctacctccaccgcctacatggaactgtccagcctgagatctgaggacaccgccgtgtactactgcgccagaaacgtgttcgacggctactggctggtgtattggggccagggaacactggtcacagtgtctagcggaggcggaggatctggtggtggtggatctggcggcggaggctctgatatcgtgatgacccagacacctctgagcctgcctgtgacacctggcgagcctgcttccatctcctgccggtcctctaagtccctgctgcactctaacggcatcacctacctgtactggtatctgcagaagcccggccagtctcctcagctgctgatctaccagatgtccaacctggtgtctggcgtgcccgacagattttccggctctggctctggcaccgacttcaccctgaagatctccagagtggaagccgaggacgtgggcgtgtactattgtgcccagaacctggaactgccctacacctttggcggaggcaccaaggtggaaatcaagagtggtggcggtggcagccaggtccagctgcaagaatctggaccaggcctcgtgaagcctagcggcacactgtctctgacctgtgctgtctctggcgtgtccatccggtccatcaactggtggaattgggtccgccagcctccaggcaaaggcctggaatggatcggcgagatctaccactccggctccaccaactacaaccccagcctgaagtcccgcgtgaccatctctgtggacaagtccaagaaccagttctccctgaagctgaactccgtgaccgccgctgataccgctgtgtattactgtgctcgcgacggcggaatcgccgtgaccgattactactactacggcctggatgtgtggggacagggcaccacagtgactgtgtctagc

SEQ ID NO: 58 - DNA, 1092 bp, Artificial Sequence
  Description of Artificial Sequence Synthetic polynucleotide
gaggtgcagctgctggaatctggcggaggattggttcagcctggcggctctctgagactgtcttgtgccgcttccggcttcaccttctccagctacgctatgtcctgggtccgacaggctcctggcaaaggactggaatgggtgtccgccatctctggctctggcggcagcacctactacgccgattctgtgaagggcagattcaccatcagccgggacaactccaagaacaccctgtacctgcagatgaactccctgagagccgaggacaccgccgtgtactactgcgctaagtcttacggcgccttcgactattggggccagggcacactggtcacagtttctagcggcggaggtggaagcggaggcggaggtagtggtggtggcggatctgacatccagatgacccagtctccatccagcctgtctgcctctgtgggcgacagagtgaccatcacctgtcaggccagccaggacatcaacaagtacctgaactggtatcagcagaagcccggcaaggcccctaagctgttgatctacggcgccagcagactggaaaccggcgtgccctctagattttccggctctggatctggcaccgactttacctttacaatctccagcctgcagcctgaggatatcgctacctactactgccagcagaaacaccctagataccctcggaccttcggacagggcaccaaggtggaaatcaagtctggcggtggtggctcccaggttcagcttgttcaatctggcgccgaagtgaagaaacccggctccagtgtgaaggtgtcctgcaaggcttctggctacgccttctcctactcctggatcaactgggttcgacaagcccctggacagggccttgagtggatgggcagaatctttcctggcgacggcgacaccgactacaacggcaagtttaagggccgcgtgacaatcaccgccgacaagtctacctccaccgcctacatggaactgtccagcctgagaagcgaggatacagctgtgtattactgtgcccggaacgtgttcgacggctactggctggtttactggggacagggaacactcgtgacagtgtctagc

SEQ ID NO: 59 - DNA, 1074 bp, Artificial Sequence
  Description of Artificial Sequence Synthetic polynucleotide
caggtgcagctggttcagtctggcgccgaagtgaagaaacctggctcctccgtgaaggtgtcctgcaaggcttctggctacgccttctcctactcctggatcaactgggtccgacaggctcctggacagggacttgagtggatgggcagaatctttcctggcgacggcgacaccgactacaacggcaagtttaagggcagagtgaccatcaccgccgacaagtctacctccaccgcctacatggaactgtccagcctgagatctgaggacaccgccgtgtactactgcgccagaaacgtgttcgacggctactggctggtgtattggggccagggaacactggtcacagtgtctagcggaggcggaggatctggtggtggtggatctggcggcggaggctctgatatcgtgatgacccagacacctctgagcctgcctgtgacacctggcgagcctgcttccatctcctgccggtcctctaagtccctgctgcactctaacggcatcacctacctgtactggtatctgcagaagcccggccagtctcctcagctgctgatctaccagatgtccaacctggtgtctggcgtgcccgacagattttccggctctggctctggcaccgacttcaccctgaagatctccagagtggaagccgaggacgtgggcgtgtactattgtgcccagaacctggaactgccctacacctttggcggaggcaccaaggtggaaatcaagagtggtggcggtggctccgagattgtgctgactcagtctcccgccacactgtctttgagccctggcgagagagctaccctgtcctgtagagcctctgagtccgtgtcctctaacctggcctggtatcagcaaaaacccggacaggccccacggctgttgatctacggcgccttcaatagagccacaggcatccccgctagattctctggctccggctccggaacagactttacactgaccatctccagcctggaacctgaggatttcgctgtgtattactgccagcagcggagcgactggttcacctttggaggcggaacaaaggtcgagatcaag

SEQ ID NO: 60 - DNA, 1071 bp, Artificial Sequence
  Description of Artificial Sequence Synthetic polynucleotide
gaggtgcagctgctggaatctggcggaggattggttcagcctggcggctctctgagactgtcttgtgccgcttccggcttcaccttctccagctacgctatgtcctgggtccgacaggctcctggcaaaggactggaatgggtgtccgccatctctggctctggcggcagcacctactacgccgattctgtgaagggcagattcaccatcagccgggacaactccaagaacaccctgtacctgcagatgaactccctgagagccgaggacaccgccgtgtactactgcgctaagtcttacggcgccttcgactattggggccagggcacactggtcacagtttctagcggcggaggtggaagcggaggcggaggtagtggtggtggcggatctgacatccagatgacccagtctccatccagcctgtctgcctctgtgggcgacagagtgaccatcacctgtcaggccagccaggacatcaacaagtacctgaactggtatcagcagaagcccggcaaggcccctaagctgttgatctacggcgccagcagactggaaaccggcgtgccctctagattttccggctctggatctggcaccgactttacctttacaatctccagcctgcagcctgaggatatcgctacctactactgccagcagaaacaccctagataccctcggaccttcggacagggcaccaaggtggaaatcaagtctggcggtggcggctccgatatcgtgatgacacagacccctctgagcctgcctgtgacacctggcgagcctgcttccatctcctgccggtcctctaagtccctgctgcactctaacggcatcacctacctgtattggtacttgcagaagcctggccagtctcctcagctgctcatctaccagatgtccaacctggtgtctggcgtgcccgacagattttctggcagcggctctggcacagatttcaccctgaagatctccagagtggaagccgaggatgtgggcgtgtactattgtgcccagaacctggaactgccctacacctttggcggcggaacaaaggtcgagatcaag

SEQ ID NO: 61 - DNA, 966 bp, Artificial Sequence
  Description of Artificial Sequence Synthetic polynucleotide
gcatccccgaccagccccaaggtcttcccgctgagcctcgacagcaccccccaagatgggaacgtggtcgtcgcatgcctggtccagggcttcttcccccaggagccactcagtgtgacctggagcgaaagcggacagggtgtgaccgccagaaacttcccacctagccaggatgcctccggggacctgtacaccacgagcagccagctgaccctgccggccacacagtgcccagacggcaagtccgtgacatgccacgtgaagcactacacgaatcccagccaggatgtgactgtgccctgccgtgttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggcagtgcccagccatggaaccatggggagaccttcacctgcactgctgcccaccccgagttgaagaccccactaaccgccactcttagtaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaa

SEQ ID NO: 62 - DNA, 966 bp, Artificial Sequence
  Description of Artificial Sequence Synthetic polynucleotide
gctagcccaacctctcctaaggtgttccctctgagcctggacagcacccctcaggatggaaatgtggtggtggcctgtctggtgcagggattcttcccacaagagcccctgtccgtgacttggagcgaatctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaatcctagccaggacgtgaccgtgccttgcagagttcctcctcctccaccttgctgtcaccctcggctgtctctgcacagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtctagtgtgctgcctggcagcgcccagccttggaatcatggcgagacattcacctgtaccgctgctcaccccgagctgaaaacccctctgaccgccacactgtccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaag

SEQ ID NO: 63 - DNA, 1014 bp, Artificial Sequence
  Description of Artificial Sequence Synthetic polynucleotide
gcatccccgaccagccccaaggtcttcccgctgagcctcgacagcaccccccaagatgggaacgtggtcgtcgcatgcctggtccagggcttcttcccccaggagccactcagtgtgacctggagcgaaagcggacagggtgtgaccgccagaaacttcccacctagccaggatgcctccggggacctgtacaccacgagcagccagctgaccctgccggccacacagtgcccagacggcaagtccgtgacatgccacgtgaagcactacacgaatcccagccaggatgtgactgtgccctgccgtgttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggcagtgcccagccatggaaccatggggagaccttcacctgcactgctgcccaccccgagttgaagaccccactaaccgccactcttagtaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacc

SEQ ID NO: 64 - DNA, 1020 bp, Artificial Sequence
  Description of Artificial Sequence Synthetic polynucleotide
gcatccccgaccagccccaaggtcttcccgctgagcctcgacagcaccccccaagatgggaacgtggtcgtcgcatgcctggtccagggcttcttcccccaggagccactcagtgtgacctggagcgaaagcggacagaacgtgaccgccagaaacttcccacctagccaggatgcctccggggacctgtacaccacgagcagccagctgaccctgccggccacacagtgcccagacggcaagtccgtgacatgccacgtgaagcactacacgaatcccagccaggatgtgactgtgccctgcccagttcccccacctcccccatgctgccacccccgactgtcgctgcaccgaccggccctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctac

SEQ ID NO: 65 - DNA, 1020 bp, Artificial Sequence
  Description of Artificial Sequence Synthetic polynucleotide
gccagccccaccagccccaaggtgttccccctgagcctggacagcaccccccaggacggcaacgtggtggtggcctgcctggtgcagggcttcttcccccaggagcccctgagcgtgacctggagcgagagcggccagaacgtgaccgccagaaacttcccccccagccaggacgccagcggcgacctgtacaccaccagcagccagctgaccctgcccgccacccagtgccccgacggcaagagcgtgacctgccacgtgaagcactacaccaacagcagccaggacgtgaccgtgccctgcagagtgccccccccccccccctgctgccaccccagactgagcctgcacagacccgccctggaggacctgctgctgggcagcgaggccaacctgacctgcaccctgaccggcctgagagacgccagcggcgccaccttcacctggacccccagcagcggcaagagcgccgtgcagggcccccccgagagagacctgtgcggctgctacagcgtgagcagcgtgctgcccggctgcgcccagccctggaaccacggcgagaccttcacctgcaccgccgcccaccccgagctgaagacccccctgaccgccaacatcaccaagagcggcaacaccttcagacccgaggtgcacctgctgcccccccccagcgaggagctggccctgaacgagctggtgaccctgacctgcctggccagaggcttcagccccaaggacgtgctggtgagatggctgcagggcagccaggagctgcccagagagaagtacctgacctgggccagcagacaggagcccagccagggcaccaccacctacgccgtgaccagcatcctgagagtggccgccgaggactggaagaagggcgagaccttcagctgcatggtgggccacgaggccctgcccctggccttcacccagaagaccatcgacagactggccggcaagcccacccacatcaacgtgagcgtggtgatggccgaggccgacggcacctgctac

SEQ ID NO: 66 - DNA, 1059 bp, Artificial Sequence
  Description of Artificial Sequence Synthetic polynucleotide
gcatccccgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgaccgtgacctctgtggctgctacagcgtgtccagtgtcctgccgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctac

SEQ ID NO: 67 - DNA, 321 bp, Artificial Sequence
  Description of Artificial Sequence Synthetic polynucleotide
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 68 - DNA, 321 bp, Artificial Sequence
  Description of Artificial Sequence Synthetic polynucleotide
cggacagtggccgctccttccgtgttcatcttcccaccttccgacgagcagctgaagtccggcacagctagcgtggtctgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 69 - DNA, 318 bp, Artificial Sequence
  Description of Artificial Sequence Synthetic polynucleotide
ggacagcctaaggccgctccatccgtgacactgttccctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgctagctcctacctgtctctgacccctgaacagtggaagtcccaccggtcctacagctgccaagtgacccatgagggctccaccgtggaaaagaccgtggctcctaccgagtgctct

SEQ ID NO: 70 - protein, 5 aa, Artificial Sequence
  Description of Artificial Sequence Synthetic peptide
NYNMH

SEQ ID NO: 71 - protein, 5 aa, Artificial Sequence
  Description of Artificial Sequence Synthetic peptide
GYGMS

SEQ ID NO: 72 - protein, 10 aa, Artificial Sequence
  Description of Artificial Sequence Synthetic peptide
GYTFTNYWIH

SEQ ID NO: 73 - protein, 10 aa, Artificial Sequence
  Description of Artificial Sequence Synthetic peptide
GYTFTNYVIH

SEQ ID NO: 74 - protein, 10 aa, Artificial Sequence
  Description of Artificial Sequence Synthetic peptide
GYSFTNYYIH

SEQ ID NO: 75 - protein, 8 aa, Artificial Sequence
  Description of Artificial Sequence Synthetic peptide
GYTFTNHV

SEQ ID NO: 76 - protein, 16 aa, Artificial Sequence
  Description of Artificial Sequence Synthetic peptide
TIYPGNDDTSYNQKFK

SEQ ID NO: 77 - protein, 17 aa, Artificial Sequence
  Description of Artificial Sequence Synthetic peptide
TITSGGTYTYYPDSVKG

SEQ ID NO: 78 - protein, 16 aa, Artificial Sequence
  Description of Artificial Sequence Synthetic peptide
YTDPRTDYTEYNQKFK

SEQ ID NO: 79 - protein, 17 aa, Artificial Sequence
  Description of Artificial Sequence Synthetic peptide
YIYPYNDGILYNEKFKG

SEQ ID NO: 80 - protein, 17 aa, Artificial Sequence
  Description of Artificial Sequence Synthetic peptide
YIDPLNGDTTYNQKFKG

SEQ ID NO: 81 - protein, 8 aa, Artificial Sequence
  Description of Artificial Sequence Synthetic peptide
IYPYNDGT

SEQ ID NO: 82 - protein, 8 aa, Artificial Sequence
  Description of Artificial Sequence Synthetic peptide
GGYRAMDY

SEQ ID NO: 83 - protein, 9 aa, Artificial Sequence
  Description of Artificial Sequence Synthetic peptide
SLAGNAMDY

SEQ ID NO: 84 - protein, 8 aa, Artificial Sequence
  Description of Artificial Sequence Synthetic peptide
GGRVGLGY

SEQ ID NO: 85 - protein, 8 aa, Artificial Sequence
  Description of Artificial Sequence Synthetic peptide
GGYYVPDY

SEQ ID NO: 86 - protein, 8 aa, Artificial Sequence
  Description of Artificial Sequence Synthetic peptide
GGKRAMDY

SEQ ID NO: 87 - protein, 10 aa, Artificial Sequence
  Description of Artificial Sequence Synthetic peptide
ARGGYYTYDD

SEQ ID NO: 88 - protein, 16 aa, Artificial Sequence
  Description of Artificial Sequence Synthetic peptide
RSSQSIVYSNGNTYLG

SEQ ID NO: 89 - protein, 11 aa, Artificial Sequence
  Description of Artificial Sequence Synthetic peptide
RASQTISDYLH

SEQ ID NO: 90 - protein, 16 aa, Artificial Sequence
  Description of Artificial Sequence Synthetic peptide
RSSQNIVQSNGNTYLE

SEQ ID NO: 91 - protein, 16 aa, Artificial Sequence
  Description of Artificial Sequence Synthetic peptide
RSRQSIVHTNGNTYLG

SEQ ID NO: 92 - protein, 11 aa, Artificial Sequence
  Description of Artificial Sequence Synthetic peptide
RASQDISNYLN

SEQ ID NO: 93 - protein, 11 aa, Artificial Sequence
  Description of Artificial Sequence Synthetic peptide
QSLVHSNGKTY

SEQ ID NO: 94 - protein, 7 aa, Artificial Sequence
  Description of Artificial Sequence Synthetic peptide
KVSNRFS

SEQ ID NO: 95 - protein, 7 aa, Artificial Sequence
  Description of Artificial Sequence Synthetic peptide
FASQSIS

SEQ ID NO: 96 - protein, 7 aa, Artificial Sequence
  Description of Artificial Sequence Synthetic peptide
KVFHRFS

SEQ ID NO: 97 - protein, 7 aa, Artificial Sequence
  Description of Artificial Sequence Synthetic peptide
YTSRLYS

SEQ ID NO: 98 - protein, 9 aa, Artificial Sequence
  Description of Artificial Sequence Synthetic peptide
FQGSHVPYT

SEQ ID NO: 99 - protein, 9 aa, Artificial Sequence
  Description of Artificial Sequence Synthetic peptide
QNGHGFPRT

SEQ ID NO: 100 - protein, 9 aa, Artificial Sequence
  Description of Artificial Sequence Synthetic peptide
QQGNTLPWT

SEQ ID NO: 101 - protein, 9 aa, Artificial Sequence
  Description of Artificial Sequence Synthetic peptide
SQSTHVPYT

SEQ ID NO: 102 - protein, 8 aa, Artificial Sequence
  Description of Artificial Sequence Synthetic peptide
GYAFSYSW

SEQ ID NO: 103 - protein, 8 aa, Artificial Sequence
  Description of Artificial Sequence Synthetic peptide
GYTFTSYN

SEQ ID NO: 104 - protein, 8 aa, Artificial Sequence
  Description of Artificial Sequence Synthetic peptide
GFTFNDYA

SEQ ID NO: 105 - protein, 8 aa, Artificial Sequence
  Description of Artificial Sequence Synthetic peptide
GFNIKDTY

SEQ ID NO: 106 - protein, 8 aa, Artificial Sequence
  Description of Artificial Sequence Synthetic peptide
GFTFTDYT

SEQ ID NO: 107 - protein, 8 aa, Artificial Sequence
  Description of Artificial Sequence Synthetic peptide
GFSLTNYG

SEQ ID NO: 108 - protein, 10 aa, Artificial Sequence
  Description of Artificial Sequence Synthetic peptide
GGSVSSGDYY

SEQ ID NO: 109 - protein, 10 aa, Artificial Sequence
  Description of Artificial Sequence Synthetic peptide
GGSISSGDYY

SEQ ID NO: 110 - protein, 8 aa, Artificial Sequence
  Description of Artificial Sequence Synthetic peptide
GYSFTGYT

SEQ ID NO: 111 - protein, 8 aa, Artificial Sequence
  Description of Artificial Sequence Synthetic peptide
GSSFTGYN

SEQ ID NO: 112 - protein, 8 aa, Artificial Sequence
  Description of Artificial Sequence Synthetic peptide
IFPGDGDT

SEQ ID NO: 113 - protein, 8 aa, Artificial Sequence
  Description of Artificial Sequence Synthetic peptide
IYPGNGDT

SEQ ID NO: 114 - protein, 8 aa, Artificial Sequence
  Description of Artificial Sequence Synthetic peptide
ISWNSGSI

SEQ ID NO: 115 - protein, 8 aa, Artificial Sequence
  Description of Artificial Sequence Synthetic peptide
IYPTNGYT

SEQ ID NO: 116 - protein, 8 aa, Artificial Sequence
  Description of Artificial Sequence Synthetic peptide
VNPNSGGS

SEQ ID NO: 117 - protein, 7 aa, Artificial Sequence
  Description of Artificial Sequence Synthetic peptide
IWSGGNT

SEQ ID NO: 118 - protein, 7 aa, Artificial Sequence
  Description of Artificial Sequence Synthetic peptide
IYYSGNT

SEQ ID NO: 119 - protein, 7 aa, Artificial Sequence
  Description of Artificial Sequence Synthetic peptide
IYYSGST

SEQ ID NO: 120 - protein, 8 aa, Artificial Sequence
  Description of Artificial Sequence Synthetic peptide
ITPYNGAS

SEQ ID NO: 121 - protein, 8 aa, Artificial Sequence
  Description of Artificial Sequence Synthetic peptide
IDPYYGGT

SEQ ID NO: 122 - protein, 12 aa, Artificial Sequence
  Description of Artificial Sequence Synthetic peptide
ARNVFDGYWLVY

SEQ ID NO: 123 - protein, 14 aa, Artificial Sequence
  Description of Artificial Sequence Synthetic peptide
ARSTYYGGDWYFNV

SEQ ID NO: 124 - protein, 15 aa, Artificial Sequence
  Description of Artificial Sequence Synthetic peptide
AKDIQYGNYYYGMDV

SEQ ID NO: 125 - protein, 13 aa, Artificial Sequence
  Description of Artificial Sequence Synthetic peptide
SRWGGDGFYAMDY

SEQ ID NO: 126 - protein, 13 aa, Artificial Sequence
  Description of Artificial Sequence Synthetic peptide
ARNLGPSFYFDYW

SEQ ID NO: 127 - protein, 13 aa, Artificial Sequence
  Description of Artificial Sequence Synthetic peptide
ARALTYYDYEFAY

SEQ ID NO: 128 - protein, 11 aa, Artificial Sequence
  Description of Artificial Sequence Synthetic peptide
VRDRVTGAFDI

SEQ ID NO: 129 - protein, 13 aa, Artificial Sequence
  Description of Artificial Sequence Synthetic peptide
ARVSIFGVGTFDY

SEQ ID NO: 130 - protein, 12 aa, Artificial Sequence
  Description of Artificial Sequence Synthetic peptide
ARGGYDGRGFDY

SEQ ID NO: 131 - protein, 6 aa, Artificial Sequence
  Description of Artificial Sequence Synthetic peptide
VSGMEY

SEQ ID NO: 132 - protein, 11 aa, Artificial Sequence
  Description of Artificial Sequence Synthetic peptide
KSLLHSNGITY

SEQ ID NO: 133 - protein, 5 aa, Artificial Sequence
  Description of Artificial Sequence Synthetic peptide
SSVSY

SEQ ID NO: 134 - protein, 6 aa, Artificial Sequence
  Description of Artificial Sequence Synthetic peptide
QSVSSY

SEQ ID NO: 135 - protein, 6 aa, Artificial Sequence
  Description of Artificial Sequence Synthetic peptide
QDVNTA

SEQ ID NO: 136 - protein, 6 aa, Artificial Sequence
  Description of Artificial Sequence Synthetic peptide
QDVSIG

SEQ ID NO: 137 - protein, 6 aa, Artificial Sequence
  Description of Artificial Sequence Synthetic peptide
QSIGTN

SEQ ID NO: 138 - protein, 6 aa, Artificial Sequence
  Description of Artificial Sequence Synthetic peptide
QDISNY

SEQ ID NO: 139 - protein, 11 aa, Artificial Sequence
  Description of Artificial Sequence Synthetic peptide
QSLVHRNGNTY

SEQ ID NO: 140 - protein, 9 aa, Artificial Sequence
  Description of Artificial Sequence Synthetic peptide
QQWTSNPPT

SEQ ID NO: 141 - protein, 8 aa, Artificial Sequence
  Description of Artificial Sequence Synthetic peptide
QRSNWPIT

SEQ ID NO: 142 - protein, 9 aa, Artificial Sequence
  Description of Artificial Sequence Synthetic peptide
QQHYTTPPT

SEQ ID NO: 143 - protein, 9 aa, Artificial Sequence
  Description of Artificial Sequence Synthetic peptide
QQYYIYPYT

SEQ ID NO: 144 - protein, 9 aa, Artificial Sequence
  Description of Artificial Sequence Synthetic peptide
QQNNNWPTT

SEQ ID NO: 145 - protein, 9 aa, Artificial Sequence
  Description of Artificial Sequence Synthetic peptide
QHFDHLPLA

SEQ ID NO: 146 - protein, 9 aa, Artificial Sequence
  Description of Artificial Sequence Synthetic peptide
HQYGSTPLT

SEQ ID NO: 147 - protein, 9 aa, Artificial Sequence
  Description of Artificial Sequence Synthetic peptide
QQWSKHPLT

SEQ ID NO: 148 - protein, 10 aa, Artificial Sequence
  Description of Artificial Sequence Synthetic peptide
SQSTHVPPLT

SEQ ID NO: 149 - protein, 9 aa, Unknown
  Description of Unknown integrin binding peptide
CYGGRGDTP